(19) United States
(12) Patent Application Publication
(10) Pub. No.: US 2012/0129684 A1
(43) Pub. Date: May 24, 2012

(54) USE OF A POROUS CRYSTALLINE HYBRID SOLID AS A NITROGEN OXIDE REDUCTION CATALYST AND DEVICES

(75) Inventors: Alexandre Vimont, Merville-Franceville (FR); Patricia Horcajada Cortes, Chaville (FR); Young Kyu Hwang, Daejeon (KR); Gerard Ferey, Paris (FR); Marco Daturi, Epron (FR); Jong-San Chang, Daejeon (KR); Christian Serre, Plaisir (FR); Ji Yoon, Seoul (KR)

(73) Assignee: CENTRE NATIONAL DE LA RECHERCHE SCIENTIFIQUE CNRS, Paris Cedex 16 (FR)

(21) Appl. No.: 13/322,321

(22) PCT Filed: May 28, 2010
(86) PCT No.: PCT/FR2010/000402

§ 371 Date: Feb. 9, 2012

(51) Int. Cl. B01D 53/56; B01J 31/22; B01D 29/00; A61L 9/00; B01D 61/00; C07F 15/02; B01D 53/94

(52) U.S. Cl. 502/170; 556/147; 423/239.1; 423/213.5; 422/177; 422/122; 210/767; 210/348

(57) ABSTRACT

The present invention relates to the use of solids consisting of a metal-organic framework (MOF) and having the units of the following formula (I): MmOkXILp as a nitrogen-oxide catalyst. The present invention also relates to devices for enabling the implementation of said use. The nitrogen oxides in question are nitrogen monoxide and nitrogen dioxide, collectively referred to as NOx. The MOF solids of the present invention are advantageously capable of removing nitrogen oxides from a liquid or gaseous effluent, for example from water, from the exhaust gases of a vehicle, factory, workshop, laboratory, stored products, urban air vents, etc., without any reducing agent and at a low temperature. The DeNOx catalysis is a major issue for our societies. The invention can be used for reducing or even avoiding the consequences for public health of the toxic NOx gases resulting from human activity.

### TECHNICAL FIELD

The present invention relates to the use of solids consisting of a metal-organic framework (MOF), as a nitrogen oxide reduction catalyst.

The solids in question are crystalline hybrid solids consisting of an ion-covalent assembly of inorganic units, for example transition metal, lanthanum, alkali metal, etc., and of organic ligands with several complexing groups, for example carboxylates, phosphonates, phosphates, imidazolates, etc. These solids usable in the present invention are defined in the present text.

The nitrogen oxides in question are nitric oxide (NO) and nitrogen dioxide (NO₂), collectively designated NOx, as well as nitrous oxide (N₂O), dinitrogen trioxide (N₂O₃) and dinitrogen tetroxide (N₂O₄).

The MOF solids of the present invention are advantageously able to remove nitrogen oxides from a liquid or gaseous effluent, for example water, the exhaust gases from a vehicle, factory, workshop, laboratory, stored products, urban air vents, etc.

“DeNOx” catalysis is a major challenge for society. It makes it possible to reduce or even avoid the health consequences of the toxic NOx gases resulting from human activity.

The present invention relates to a new family of DeNOx catalysts that offers the enormous advantage of converting NOx even at room temperature and in the absence of reducing species, which constitutes a major advance in this area.

The references in square brackets [X] refer to the list of references given after the examples.

### PRIOR ART

Metal-organic frameworks (MOFs) are coordination polymers with an inorganic-organic hybrid framework comprising metal ions and organic ligands coordinated to the metal ions. These materials are organized in one-, two- or three-dimensional frameworks where the metal clusters are joined together periodically by spacer ligands. These materials have a crystalline structure, most often are porous and are used in numerous industrial applications such as gas storage, adsorption of liquids, separation of liquids or of gases, catalysis, etc.

Following World War II and in parallel with the “thirty glorious years” [of French prosperity between 1945 and 1973], the automobile soon became the primary means of locomotion in the industrialized countries. Under the effect of economic growth, the total number of automobiles is constantly increasing. In France, the number of private cars rose above 16.7 million in 1975, reaching about 36 million at the end of 2005. On a global scale, due mainly to the emergence of the developing countries, it is also expected to double by 2030, as can be seen in the document “Definition and implications of the private car concept”, on www.senat.fr/rap/r05-125/r05-125.html [1].

This increased mobility throughout the world unfortunately has an impact on the environment and on health. Heat engines (diesel, gasoline and LPG) notably account for a substantial proportion of atmospheric pollution. The main pollutants emitted are oxides of carbon (CO and CO₂), volatile organic compounds (VOCs), unburnt hydrocarbons, and nitrogen oxides. This term essentially comprises nitric oxide (NO) and nitrogen dioxide (NO₂), together with smaller amounts of nitrous oxide (N₂O), dinitrogen trioxide (N₂O₃) and dinitrogen tetroxide (N₂O₄). The compounds analyzed by the monitoring networks are only NO and NO₂, the sum of which is covered by the term NOx.

Today, numerous studies have been able to demonstrate the harmful effects caused and to raise people's awareness. One example of these studies can be found on www.doctissimo.fr/html/sante/mag_{—}2001/mag0817/dossier/sa_{—}4404_pollution_effets_sante.htm, “Pollution: What effects on health?” [2]. According to the World Health Organization, about 3 million people die every year from the effects of atmospheric pollution.

Nitrogen oxides (NOx) are mainly emitted by motor vehicles and industry. They are formed in the combustion chamber by a high-temperature chain reaction between oxygen, the nitrogen of the air and hydroxyl radicals, as described in the document P. Degobert, Automobile et Pollution, Editions Technip, Paris (1992) “Zeldovich reaction” [3]). These gases, which have toxic effects on health, as described in Samoli E et al., Eur. Respir. J., 27 (2006) 1129; Peters A et al., Epidemiology (Cambridge, Mass.), (2000 January) Vol. 11, No. 1, p. 11; and Arden Pope C et al., Circulation, 109 (2004) 71 [4, 5, 6] and are harmful to the environment, are responsible in particular for the formation of photochemical smog, tropospheric ozone and some acid rain. They are becoming increasingly present in city air and are therefore monitored [3].

Although much effort has been expended for removing these pollutants, using systems for reduction of NOx at the sources of emission (vehicles, smokestacks, etc.), increasing concentrations of nitrogen oxides are being recorded in cities, as described in the document Gouriou F et al., Atmospheric Environment, 38 (2004) 2831 [7]. This is due to the increasing use of diesel engines and stratified-charge engines (“lean burn engines”), which are fundamentally less polluting and less costly in terms of fuel, but they operate with combustion mixtures that are very rich in oxygen, therefore promoting the production of nitrogen oxides.

Moreover, the massive use of catalysts based on precious metals in post-combustion systems, in order to remove CO, unburnt hydrocarbons and soot from vehicle emissions, has further increased the NOx content of the air of large conurbations, near main highways and, especially, in the passenger compartments of cars, as described in Son B et al., Environmental Research, 94 (2004) 291; and Praml G et al., International Archives of Occupational and Environmental Health, 73 (2000) 209 [8, 9].

Authorities' awareness of the nuisance caused by these various emissions in the atmosphere led them to establish standards aiming to reduce these emissions. The first European directives appeared in 1970, and have since been strengthened and regularly revised by the EURO standards. At present, the commission proposes EURO standards V and VI, the values of which are supposed to come into force on 1 Sep. 2009 for the first and 5 years later for the next (see Table A below: European regulations relating to emissions from new vehicles. Values in g/km; ADEME data published on website www.ademe.fr [10]).

EURO 5 places main emphasis on particulate emissions from diesel exhausts, which will make the use of filters on these vehicles indispensable, whereas for the next standard it will be in particular nitrogen oxides that will have to be reduced. This is because no existing technology guarantees effective action for reducing NOx to the values recommended for EURO 6. It is hoped that progress will have been made by the 2014 deadline. This last-mentioned directive is still under investigation by the European Community.

TABLE A
European regulations relating to emissions
from new vehicles. Values in g/km

| | GASOLINE | | | | DIESEL | | | |
|---|---|---|---|---|---|---|---|---|
| | | | | HC + | | | HC + | Partic- |
| | CO | HC | NOx | NOx | CO | NOx | NOx | ulates |
| Euro 1 | 2.72 | — | — | 0.97 | 2.72 | — | 0.97 | 0.14 |
| (1992) | | | | | | | | |
| Euro 2 | 2.20 | — | — | 0.5 | 1.00 | — | 0.90 | 0.10 |
| (1996) | | | | | | | | |
| Euro 3 | 2.30 | 0.20 | 0.15 | — | 0.64 | 0.50 | 0.56 | 0.05 |
| (2000) | | | | | | | | |
| Euro 4 | 1.00 | 0.10 | 0.08 | — | 0.50 | 0.25 | 0.30 | 0.025 |
| (2005) | | | | | | | | |
| Euro 5 | 1.00 | 0.075 | 0.06 | — | 0.50 | 0.18 | 0.23 | 0.005 |
| (2009) | | | | | | | | |
| Euro 6 | | | | — | 0.50 | 0.008 | 0.17 | 0.005 |
| (2014) | | | | | | | | |

The problem is framed differently depending on whether we are considering vehicles with spark ignition, i.e. using gasoline, or with indirect injection such as when using diesel engines.

Operating with a large excess of air, the latter are more economical than gasoline engines, which require stoichiometric air/fuel ratios: 1 gram of fuel to 14.7 grams of air for gasoline versus about 30 grams for diesel. These highly oxidizing conditions for the diesel engine enable it to have lower emissions of CO and hydrocarbons. Moreover, a smaller proportion of NOx forms owing to a lower combustion temperature. However, diesel vehicles produce significant amounts of particulates, in contrast to vehicles using gasoline.

Since the beginning of the 1980s, progress has been made in connection with polluting emissions, with the exception of CO₂ (see for example Miyata H et al., J. Chem. Soc., Faraday Trans., 91 (1995) 149 [11]. They are linked to various factors such as improvement in engine control, better quality of gasoline (in 1989, the appearance of lead-free gasoline in France/gradual removal of sulfur), as well as use of the first catalytic converters.

Efforts were first directed at vehicles using gasoline, as they are fundamentally more polluting. The first catalytic converters appeared starting from 1975, as reported notably in Shelef M et al., Catal. Today, (2000) 35 [12] and, since the beginning of the 1990s, they are provided on all engine vehicles in the Western hemisphere, with the name three-way catalyst (TWC), as they simultaneously provide oxidation of CO and of hydrocarbons to CO₂, as well as reduction of NOx, as described in Farrauto R J et al., Catal. Today, 51 (1999) 351 [13].

With regard to diesel engines, their lower overall level of emission of pollutants enabled them to meet the European standards of 1993 using simple engine adjustments. Nevertheless, with the legislation becoming more stringent with EURO standard 3, they have had to be equipped with a two-way catalyst for reducing emissions of CO and of hydrocarbons, but not the emissions of nitrogen oxides. However, since 2005 the authorities have pointed the finger at nitrogen oxide emissions. Now, although the highly oxidizing gas flows in diesel engines easily allow catalytic oxidation of reducing pollutants, they make the reduction of nitrogen oxides very complicated in such an environment.

“Three-way” catalysis, operating in vehicles using gasoline, cannot be applied in a strongly oxidizing environment, as it requires stoichiometry between the oxidizing and reducing agents, which would mean diesel engines losing all their advantages, as they are well known as having lower fuel consumption. Moreover, the temperature window for the operation of TWC catalysts, suitable for vehicles using gasoline, is also too high relative to the temperature of the diesel effluents.

Various strategies have been adopted for tackling these problems, but to date, no technology is able to offer a proper response to this problem, which represents an important challenge for many scientists, as described in Jobson E., Top. Catal., 28 (2004) 191 [14]. These strategies are: 1. Selective catalytic reduction of NOx (SCR); 2. Trapping of nitrogen oxides: NOx-trap method (or NSR); and 3. Direct decomposition

### 1. Selective Catalytic Reduction of NOx (SCR)

As we have already emphasized, the low content of reducing agents in the exhausts of diesel and lean burn vehicles makes it difficult to convert NOx to nitrogen. That is why some researchers have envisaged adding one or more reducing agents to the post-treatment mixture, in the presence of a suitable catalyst for promoting this reaction. The reducing agents can be CO, whether or not combined with hydrogen, hydrocarbons or ammonia (NH₃ formed in situ from urea).

Catalytic reduction by CO or H₂: the use of two reducing agents such as CO and H₂, which are already present in the exhaust gases, has aroused quite particular interest. Notably the reaction between NO and CO, both of which are undesirable in exhausts, came immediately to mind: NO+CO→CO₂+½N₂. As for hydrogen, it can be derived in particular from the hydrocarbons or from a reaction of water gas actually in the catalyst. In both cases, catalysts based on precious metals are among the most active. A great many metal oxides (in particular perovskites) as well as zeolites exchanged with transition metals have also been studied, as described in Parvulescu V.1 et al., Catal. Today, 46 (1998) 233 [15].

Most of the mechanisms proposed can be summarized as dissociation of NO, followed by reaction between O_{ads} with CO or hydrogen to form CO₂ and H₂O. However, these reactions are by no means selective and undesirable secondary reduction products are formed. The use of hydrogen on various metal oxides notably reveals often considerable production of NH₃, as described in Shelef M et al., Ind. Eng. Chem. Prod. Res. Dev., 13 (1974) 80 [16]. The studies of Burch on Pt/SiO₂ or Pt/Al₂O₃ reported in Burch R et al., Appl. Catal. B, 23 (1999) 115 [17] give interesting results in the NO/H₂ reaction, but in real conditions, and especially at low temperature in the presence of steam, there is formation of N₂O. Moreover, as in the case of decomposition, the high oxygen content encountered in diesel exhausts inhibits the reaction. Therefore this type of application is not conceivable in diesel conditions.

Selective catalytic reduction by hydrocarbons: SCR of NO by the hydrocarbons present at the outlet from diesel and lean burn engines is an interesting way of removing nitrogen oxides. However, the low content of this reducing agent in exhausts (about 2000 ppm of carbon equivalent), as well as the high concentration of oxygen, represent a real challenge for methods of this type. As with the studies mentioned above, a great many catalytic materials have also been tested, but no satisfactory solution has been found to date.

In 1990, Held et al., SAE paper No. 900496, (1990) [18] as well as Iwamoto, Proc. Meet. Catal. Technol. Renoval of NO, Tokyo, (1990) 17 [19] discovered separately that alkanes, as well as alkenes, are able to reduce nitrogen oxide on a zeolite Cu-ZSM-5, not only in the presence of oxygen, but also that in excess of oxygen this reaction is promoted, at least with respect to the diesel emission temperatures. Busca et al., as stated in J. Catal., 214 (2003) 179 and Appl. Catal. B: Environ., 71 (2007) 216 [20, 21] also investigated various materials, notably in the presence of methane. Reduction is optimal around 573 K; beyond this temperature, oxidation of the hydrocarbon is promoted at the expense of reduction of NO. The optimal degree of exchange of copper on zeolite ZSM-5 is close to 100% (Sato S et al., Appl. Catal., 70 (1991) L1 [22]), which gives a reduction activity 5 times greater than on HZSM-5. The nature of the reducing agent as well as its concentration have also been investigated. In fact, although ethylene, propane, propylene or butane lead to reduction to nitrogen even in the presence of water, hydrogen, carbon monoxide or methane react essentially with oxygen.

The addition of Pt to ZSM-5 makes it possible to obtain good results, notably in the presence of water without any deactivation, in contrast to Cu-ZSM-5 or Fe-MOR (Hirabayashi H et al., Chem. Lett., (1992) 2235 [23]). Nevertheless, this addition of Pt leads to the formation of N₂O in large amounts. Hamada et al. (Appl. Catal., 64 (1990) L1 and Catal. Lett., 6 (1990) 239 [24, 25]) also found, very shortly after the first discoveries with these materials, that protonated zeolites and aluminas are also active for this reaction, but the temperatures required to achieve satisfactory selectivity are much too high and the cost of the zeolites is too high.

Numerous studies have been conducted on precious metals supported on metal oxides, in particular on platinum. Burch et al. notably investigated a series of catalytic materials composed of Pt deposited on an Al₂O₃ support, with variable content of Pt and prepared from different precursors. The results reveal interdependence between the amount of metal, the temperature of maximum conversion of NO and the level of activity as described in Burch R et al., Appl. Catal. B, 4 (1994) 65 [26]. For a given precursor of Pt, when the content of metal introduced increased, they found a decrease in temperature corresponding to the maximum conversion of NOx as well as a corresponding increase in activity. However, the selectivity of these materials for nitrogen is not total: at low temperatures, large amounts of N₂O (40%) are also produced. Burch completed his investigations on Pt/Al₂O₃ with Watling (Burch et al., Appl. Catal. B, (1997) 207 [27]), introducing different elements individually: K, Cs, Mg, Ca, Ti, Co, Cu, Mo, La, Ce, via the introduction of nitrate or acetate salts. Only Ti and Mo appear to provide a slight positive effect, whereas all the others lead to a drop in activity of Pt/Al₂O₃. Precursors based on other precious metals were also tested, for example Ag, Au, Pd and Rh, but the results are barely more conclusive, though with a slight improvement with Ag. Thus, none of these promoters gives any appreciable improvement in the selectivity of the Pt/Al₂O₃ catalyst for N₂.

Other investigations of precious metals supported on alumina were reported by Obuchi et al., App. Catal. B: Environ., 2 (1993) 71 [28]. This research showed that for a given reducing agent there is indeed a relation between the activity of the catalyst and the nature of the precious metal. Regarding iridium (Ir) and palladium (Pd), they do not give conversion greater than 25%, whereas rhodium (Rh), ruthenium (Ru) as well as platinum (Pt) enable far higher values to be attained. A maximum conversion of about 60% is obtained at relatively low temperature (523 K) on Pt, whereas this same maximum is reached at 593 K, i.e. 320° C., on other precious metals, for example Rh and Ru. However, selectivity for nitrogen is very different between these metals: it is only 32% for Pt at 523 K, i.e. 250° C., against about 80% at 593 K on the others (Bamwenda G. R et al., Appl. Catal. B: Environ., 6 (1995) 311 [29]). Among these various metals supported on alumina, Rh appears to be the most selective for nitrogen.

The cost of these precious metals and the environmental impact in terms of waste generated are, however, prohibitive for laboratory, industrial and automotive applications.

The importance of the choice of reducing agent is also demonstrated by data obtained by Bourges et al. Catalysis and Automotive Pollution Control IV, Stud. Surf. Sci. Catal., 116 (1998) 213 [30], as well as by Burch and Ottery Appl. Catal. B, 9 (1996) L19 [31]. This can be seen clearly with toluene, which gives rise to good selectivity for N₂ without forming N₂O. Toluene is, however, very toxic.

Several mechanisms, which we shall not describe in detail here, have been proposed to explain the catalytic reduction of nitrogen oxides on these materials. In the course of these investigations, Burch's team [26] identified the crucial role of the surface reduction of Pt. Completely reduced on Pt/Al₂O₃, very few NOx are desorbed in the form of N₂O, as dissociation of NO on the reduced particles leads preferentially to recombination to N₂— whereas on an oxidized surface, most NOx are adsorbed and desorbed without dissociation. Moreover, the use of alkenes, in contrast to CO, potentially makes it possible to remove more surface oxygen atoms, 9 for propylene if combustion is complete against only 1 for CO, making it possible to fix and then dissociate NO. When the temperature is increased, adsorption and dissociation of NO are facilitated, as well as the mobility of N_{ads} allowing easier recombination to N₂. In contrast, for low temperatures the dissociated species react with NO to form N₂O. These results are not satisfactory.

Other researchers assume that it goes via intermediates of the type CₓH_{y}O_{z}N, which can be nitro, nitrite or carbonyl species for Tanaka et al., Appl. Catal. B: Environ., 4 (1994) L1. [32] or isocyanates or cyanide for Bamwenda et al. [29].

The simple metal oxides such as Al₂O₃, SiO₂—Al₂O₃, TiO₂, ZrO₂ or MgO, with the exception of silica alone, are active for selective catalytic reduction of nitrogen oxides by hydrocarbons in an oxidizing environment. Their performance can also be improved by adding one or more transition metals. A large number of studies have been undertaken in this direction and are reported in the literature.

For example, addition of Cu on alumina notably made it possible to improve the performance of this oxide, lowering the temperature of maximum conversion while increasing the percentage activity, as reported by Torikai Y et al., Catal. Lett., 9 (1991) 91 [33]. Similar results on this oxide were also found by adding Co or Fe, as well as on a mixed material SiO₂—Al₂O₃. Hamada et al. also compared the activities of several metal oxides (Cu, Co, Ni, Mn, Fe) supported on alumina or silica (Hamada H et al., Appl. Catal., 75 (1991) L1 [34], Inaba M et al., Proc. 1st Int. Cong. On Environ. Catal., (1995) 327 [35]) and found that catalysts based on silica are less active than those containing alumina. The activity of the latter depends on the method of preparation as well as on the thermal treatment. Moreover, catalysts containing aluminates prove to be even better. Finally, they suggest that oxidation of NO to NO₂ is the first step of the mechanism of reduction on catalysts of this type.

The works of Miyadera on several oxides based on transition metals (Cu, Co, Ag, V, Cr) supported on alumina described in Miyadera T, Appl. Catal. B, 2 (1993) 199 [36], show that for these, Ag is the most reactive in diesel conditions. It makes it possible to obtain a conversion of about 80% at 673 K. On this material (Ag/Al₂O₃), Shimizu et al. also investigated the influence of the nature of the hydrocarbon (Shimizu K et al., Appl. Catal. B: Environ., 25 (2000) 239 [37]): the activation temperature decreases with the length of the carbon chain, with total conversion of NO to N₂ at 623 K with 750 ppm of n-octane and 1000 ppm of NO for 10% oxygen and 2% water. With these catalysts, compounds such as ethanol or acetone are more effective than propylene as reducing agent, notably at lower temperatures (Hamada H et al., Appl. Catal. A: General, 88 (1992) L1 [38]). However, addition of alcohol for an automotive application requires addition of a tank, which is not very practical and in particular is uneconomic. Moreover, all these metals are toxic and they pose considerable problems for the environment and for recycling of the materials used.

However, the majority of these oxide-based catalytic systems, using alkanes or alkenes as reducing agent, suffer a significant drop in activity in the presence of water and SO₂, in contrast to those that use alcohols. It is therefore necessary to replace them frequently, which is complicated and expensive. Tabata et al. observed that addition of tin to alumina improves the resistance of these catalysts in the presence of SO₂ but also of water. Moreover, Haneda et al. Catal. Lett., 55 (1998) 47 [39] envisage instead a promoter effect of water for reduction of NO in the presence of propylene on In₂O₃/Ga₂O₃/Al₂O₃. This effect could be explained by the fact that water would lead to removal of carbon deposits from the surface of the catalyst. These solutions do not, however, solve all the problems encountered in these systems.

Catalytic reduction by ammonia (NH₃) is described as selective, in contrast to that using CO or H₂, as the reducing agent (NH₃) reacts preferentially with NO, despite the presence of oxygen in excess.

The materials that are most active for this reaction are oxides based on vanadate and possibly molybdate and tungstate supported on titania (Busca G et al., Appl. Catal. B: Environ., 18 (1998) 1 [40] and Catal. Today, 107-108 (2005) 139 [41]). Zeolites exchanged with transition metals, precious metals or activated charcoal also display activity [15].

The balancing equations of the classical reactions of reduction of NOx by NH₃ are presented below, but there are also parallel reactions leading to oxidation of NH₃ to NO or N₂O [18].

4NO+4NH₃+O₂→4N₂+6H₂O

2NO₂+4NH₃+O₂→3N₂+6H₂O

NO+NO₂+2NH₃→2N₂+3H₂O

For several years already, this method of removing nitrogen oxides by the use of ammonia, although expensive, provides very encouraging results on an industrial scale, i.e. effectiveness of 90% when the gases are within the temperature window of the catalyst: between 473 K and 773 K, i.e. about 200 to 500° C. It is operational and has been implemented in fixed installations, such as factories producing nitric acid, thermal power stations or in incinerators. But it poses various problems in connection with application for emissions with temperatures of the gases of the order of 393 to 423 K, i.e. about 120 to 150° C., for example for cement works and glassworks, which therefore need to be heated, which involves considerable additional energy costs, as well as with regard to automobiles or other mobile sources. The catalysts in fact display considerable thermal instability and injection of ammonia is difficult to control: insufficient flow or surplus, with consequent losses. These drawbacks are very problematic and a solution has not yet been found.

Numerous investigations are currently at various stages, with the aim of adapting this method to diesel vehicles. None is showing promise at present. The reducing agent envisaged is not ammonia but an aqueous solution of urea (NH₂CONH₂), odorless and nontoxic, which when injected into the exhaust will release ammonia by a hydrolysis reaction.

In a device for selective catalytic reduction of NOx, using ammonia formed from urea as reducing agent, the oxidation catalyst placed upstream makes it possible to increase the NO₂/NO ratio of the exhaust gases and thus increase the conversion efficiency notably at low temperature, bearing in mind that the reaction of NO₂ with NH₃ is quicker than the reaction of NO with NH₃; nevertheless, the presence of NO is still indispensable. In various works, an optimal ratio of NO to NO₂ was in fact determined for increasing the activity and nitrogen selectivity of this SCR reaction (Heck R. M, Catal. Today, 53 (1999) 519 Koebel M et al., Catal. Today, 53 (1999) 519 [43], Richter M et al., J. Catal., 206 (2002) 98 [44]). Moreover, a “clean up” catalyst has to be installed downstream of this device, for treating any discharges of excess ammonia, notably during the transitional phases. In fact, use of this method can lead to a salting out of ammonia.

Development of the system for automotive applications will require very precise calibration of the amount of urea injected in relation to the amount of NOx emitted by the engine, which itself depends on the exhaust temperature and the characteristics of the catalyst used. Development is therefore proving very complex and of uncertain result. In this connection, the presence of the clean-up catalyst offers additional flexibility and makes it possible to achieve higher degrees of conversion of NOx without reemission of ammonia to atmosphere. However, this results in additional cost and a more complicated device.

In the absence of oxygen, reduction of NO in the presence of NH₃ is also possible but is much slower and does not occur in a lean mixture.

6NO+4NH₃→5N₂+6H₂O

However, Kato et al. J. Phys. Chem., 85 (1981) 4099 [45] showed that with an equimolar ratio between NO and NO₂, the reaction rate is increased considerably (fast SCR)

NO+NO₂+2NH₃→2N₂+3H₂O

Thus, it is preferable to have a stoichiometric mixture of NO and NO₂ and in particular avoid an increase of NO, which reacts less rapidly with ammonia. However, depending on the operating conditions, undesirable reactions that consume ammonia may occur, notably its oxidation to nitrogen above 673 K, as pointed out by Richter et al. [44] and Satterfield C. H, “Heterogenous Catalysis in Industrial Practice” Second edition. McGraw-Hill (1991) [46], or its oxidation to NO or NO₂:

4NH₃+3O₂→2N₂+6H₂O (for T>673K)

4NH₃+5O₂→4NO+6H₂O

4NH₃+7O₂→4NO₂+6H₂O

4NH₃+4NO+3O₂→4N₂O+6H₂O

Various mechanisms have been published for explaining the phenomena observed: the works of Busca et al. [40] review various studies with vanadium catalysts supported on metal oxides.

This very efficient concept, initially envisaged and used on fixed installations, owing to the thermal stability as well as the space velocities, some years ago still did not seem to be applicable to mobile sources. In fact, the use of on-board toxic products such as ammonia and vanadium is not without problems; not to mention the low thermal stability of supports based on titania. Technological advances, including increasing the distance of the SCR catalyst from the engine, resulting in a lower temperature of the gases to be treated, as well as the use of urea as starting reactant, have partly answered these concerns. Thus, heavy trucks have recently begun to be equipped with this device for reducing their emissions of nitrogen compounds.

However, it is still difficult to apply on cars which have, in contrast to trucks, power units with constantly changing conditions and therefore with very variable amounts of NOx emitted during these transient phenomena. Improvements may still be possible through upstream installation of a very accurate system for monitoring the emissions of NOx from the engine, and thus sending the appropriate amount of urea to prevent emission of NH₃ into the atmosphere.

Nevertheless, even if these improvements see the light of day, the cost of installing such a device will probably be a major obstacle. It will in fact be necessary to equip vehicles with an additional tank with a capacity of about twenty liters and install a system for distribution of urea, which is required for the proper functioning of this system. Freezing of the urea solution is also a minor obstacle that has to be overcome for applications in cold climates. In particular, it is completely inoperable below 473 K, i.e. about 200° C.

### 2. Trapping of Nitrogen Oxides: NOx-Trap Method (or NSR)

Trapping of NOx, which allows efficiencies to be achieved comparable to those of SCR with urea, without the drawback of on-board installation of an additional reducing agent, represents a real energy-efficient, environment-friendly alternative for the treatment of nitrogen oxides and is currently being applied in many developments.

The new type of catalytic converter called NOx-trap (or NSR) appeared at the beginning of the 1990s on the initiative of the company Toyota (Toyota Patent EP 573 672A1 (1992) [47], Miyoshi N et al., SAE Technical Papers Series No. 950809, 1995 [48]). The materials used are generally compounds of a support based on alumina, ceria, or even zirconia, on which the following are deposited successively: an alkali-metal or alkaline-earth oxide (commonly Ba or Sr) performing the role of adsorbent, as well as one or more precious metals (Pt/Pd/Rh).

A special feature of this system is that it operates alternately under oxidizing and then reducing atmosphere. In fact, to make up for the surplus of fuel that would be caused by continuous reduction of nitrogen oxides in an oxygen-rich environment, it was decided first to concentrate the nitrogen oxides on the material before reducing them to nitrogen during localized injection of fuel.
For most of the time, with a flow that has a low concentration of reducing agents, the nitrogen oxides are oxidized to nitrates and stored on the adsorbent as they leave the engine.
Then periodically, during the rich phase, a larger amount of reducing agent, corresponding to a peak of fuel, is injected. This leads to decomposition of the species stored on the adsorbent, then reduction of NOx to nitrogen on the adjacent metal sites.

The removal from storage and reduction of the nitrogen oxides therefore require operation at a richness of the air/fuel mixture (λ) less than or equal to 1, which is unusual for a diesel engine. This operation is obtained by altering the engine settings, notably the air flow rate, the phasing and the duration of the injections, etc.

The objective of the developments that are in progress is to optimize these variations in richness, in order to achieve the best compromise between NOx and overconsumption of fuel. To do this, a great many parameters have to be taken into account, notably the materials used for catalyzing the reactions involved. The catalyst must in fact display optimal properties in the conditions of diesel exhausts, so as to permit good storage of the nitrogen oxides as well as good regeneration under rich flow, thus limiting fuel consumption.

Following the development by Toyota of the first NOx-trap catalyst with good performance, with the formulation: Pt—Rh/BaO/Al₂O₃, numerous improvements have been made. This method is currently validated and operational in certain countries, notably in Japan, where fuels have very low sulfur contents. In fact, the presence of sulfur leads to the formation of sulfates, which compete directly with the nitrates for the same storage sites and which, in addition, gradually saturate the material.

More recently, the company Daimler Benz (Krutzsch B et al., SAE Technical Papers Series No. 982592 (1998) [49]; German Patent No. 4319 294 (1994) [50]) developed a noncatalytic variant of this method, called “Selective NOx recirculation” (SNR). It retains the principle of alternating operation described above. However, the NOx produced during decomposition of the stored species under lean flow are not in this case reduced on precious metals, but are returned to the combustion chamber for removal: the thermodynamic equilibrium 2 NO═N₂+O₂, which is a function of the combustion temperature, ensures that most of the nitrogen oxides are reduced.

Research into the NOx-trap method is already well advanced and many works have made it possible to understand and highlight certain parameters and mechanisms that govern the activity of the materials used.

However, optimization of the complete system both at the level of the engine and of the material is still essential, to achieve reduction efficiencies that meet the new specifications, while minimizing the overconsumption of fuel caused by this concept. The introduction of this method in most European countries also requires solving the problems connected with sulfur, even if much progress has already been made in the area of fuel desulfurization, notably by the possibility of obtaining fuels at the pump having a sulfur content below 10 ppm.

### 3. Direct Decomposition

The process of direct decomposition is the easiest way to convert NOx to N₂ and O₂. Moreover, the fact that reducing agents such as CO, H₂ or hydrocarbons are not added prevents the formation of secondary pollutants such as CO, CO₂ or NH₃, except N₂O, as well as considerable savings in terms of the use of fuel in post-combustion and process engineering. At ambient pressure and temperature, NO is unstable, and its reaction of decomposition, as well as that of NO₂, is thermodynamically favorable (Glick H et al., J. Chem. Eng. Prog. Ser., 27 (1971) 850 [51]). However, from the kinetic standpoint it is inhibited by an energy of activation of NO that is much too high, of the order of 364 kJ mol⁻¹, making it metastable:

NO→½N₂+½O₂ Δ_{f}G°=−86 kJ mol⁻¹

A catalyst allowing this activation energy to be lowered without addition of co-reactant is therefore indispensable (Fritz A et al., Appl. Catal. B, 13 (1997) 1 [52], Gomez-Garcia M. A et al., Environment International 31 (2005) 445 [53]).

Numerous materials have been tested for catalyzing this decomposition reaction ([15, 52] Iwamoto et al., Catal. Today, 10 (1991) 57 [54]), such as precious metals, metal oxides or zeolites.

The result is that, whatever catalyst is used, the oxygen poisons the materials, thus leading to strong inhibition of the reaction.

Among the precious metals, Pt is the most active, and has therefore received most study [52, 54]. The mechanism of dissociation is as follows (French Patent No. 98 05363 [55]):

NO_{ads}→N_{ads}+O_{ads}

2N_{ads}→N₂

2O_{ads}→O₂

However, there is very limited desorption of oxygen. Thus, below 773 K, i.e. about 500° C., the adsorbed oxygen poisons the surface. Moreover, studies have shown that regardless of the temperature, the reactivity is negligible in the presence of 5% oxygen in the gas phase.

This technique is therefore inapplicable in diesel or “lean burn” exhausts which have an O₂ content approaching 10%, or in emissions from smokestacks, having oxygen concentrations close to 20%, corresponding to the content in the atmosphere.

As for metal oxides, they have the same limitation due to the O₂ desorption step. According to Hamada et al. this inhibition could be reduced by selecting a suitable promoter (Hamada et al. Chem. Lett., 1991, 1069. [56]). Thus, introduction of Ag by precipitation or co-precipitation into a catalyst such as Ag—CO₂O₃ makes it possible to increase both the activity and the resistance to oxygen poisoning.

Nevertheless, the activity of the Ag/Co mixed compound with a ratio of 0.05, which is the most active, is halved in the presence of 5% O₂ at 773 K, whereas Co₂O₃ has no activity in this case. This increase observed with silver could be due to its low affinity for O₂. More detailed information on the functioning of these metal oxides can be found in the works by Winter, who carried out a complete study on about forty materials (Winter E. R. S, J. Catal., 22 (1971) 158 [57]). It was shown in this work that the reaction of decomposition of NO has great similarity with that of N₂O, especially the first step of adsorption of NO on two sites of adjacent anionic vacancies. Moreover, a kinetic study revealed strong inhibition of the reaction on these solids by oxygen. The parameters and the rate of decomposition of NO largely depend on the oxygen desorption step. The authors also determined the activation energies, giving: CuO>Rh₂O₃>Sm₂O₃>SrO as the most active oxides.

Most studies on the decomposition of NO have been conducted on supported or bimetallic or alloy Pt catalysts. Kinetic studies of this reaction on these types of catalysts with observations of parameters such as the NO and O₂ partial pressure revealed the following (generally accepted) mechanism: a first step of adsorption of NO and simultaneous dissociation of this molecule to adsorbed nitrogen and oxygen. Then, desorption of these atoms, which thus release sites for a new adsorption. The oxygen desorption step is very temperature-dependent. It is difficult below 773 K. Moreover, with a gradually increasing degree of coverage of the catalyst surface with oxygen, the activity decreases until the catalyst is completely inactive.

Amirnasmi et al. J. Catal., 30 (1973) 55 [58] showed that the decomposition is of first order with respect to NO and of negative order with respect to O₂; the reaction is therefore poisoned by oxygen. This can be explained by the impossibility of reduction of Pt under the oxidizing flow, which is then inactive for decomposition.

Moreover, M. Sato, Surf. Sci., 95 (1980) 269 [59] points out that W in the reduced state is sufficiently active for the decomposition of NO but that it is difficult to obtain cations in a low oxidation state when the metal is well dispersed and isolated on the surface of an alumina. Moreover, more recently, A. M. Sica et al., J. Mol. Cat. A: Chem., 137 (1999) 287 [60] prepared the Pd-W/γAl₂O₃ complex by photochemical reaction and showed that the latter suppresses the Lewis acidity of alumina and that the resultant interaction between Pd and W modifies the properties of chemisorption of Na relative to those observed on Pd and W monometallic catalysts. Consequently, the bimetallic catalyst displays an increase in activity of decomposition of Na [60]. Finally, following the same reasoning, strong interaction between palladium and vanadium was demonstrated for the Pd-VOx/γAl₂O₃ catalyst by Neyertz et al. The reducibility of VOx is then increased and more V⁴⁺ sites are formed. These species appear to make Pd more active in the decomposition of NO, as described in C. Neyertz et al., Catal. Today, 7 (2000) 255 [61].

The oxides of the perovskite type have also been investigated for this reaction, and works have shown that, owing to their structural defects, these solids permit easier desorption of oxygens from the core. Another advantage is the stability of these catalysts. However, they only display satisfactory activities for temperatures above 900 K, i.e. 627° C., which condemns them to be unsuitable for automotive use.

The catalysts that are most active for this direct decomposition are still the copper zeolites of the type Cu-ZSM-5, investigated in particular by Iwamoto's team [54] (Iwamoto M et al., J. Chem. Soc., Faraday Trans. 1, 77 (1981) 1629) [62]. On account of their properties of adsorption-desorption of O₂ (Iwamoto M et al., J. Chem. Soc, Chem. Commun., (1972) 615 [63]), they are less inhibited by the latter.

However, in the presence of a large amount of oxygen, their activity is still insufficient, and furthermore they are particularly unstable in real conditions, especially in the presence of steam and at high temperatures, and can sometimes even cause the structure to collapse.

There is therefore a real need for an alternative catalyst that does not have the numerous drawbacks of those of the prior art, notably those mentioned above, and notably displays a strong DeNOx catalytic power, activity at low temperature, preferably at room temperature, activity in the absence of reducing agents, very good recycling properties, low cost and very good thermal and chemical stability in the conditions of use.

Moreover, there is a real need for a device for removing nitrogen oxides that does not have the drawbacks of the devices of the prior art, notably those mentioned above.

### DESCRIPTION OF THE INVENTION

The aim of the present invention is precisely to respond to these needs and drawbacks of the prior art by using particular MOFs as a catalyst for reduction of nitrogen oxide.

In particular, the present inventors discovered completely unexpectedly that a porous crystalline MOF solid comprising or consisting of a three-dimensional succession of units corresponding to the following formula (I):

MₘOₖXₗLₚ (I)

where, in formula (I):
each occurrence of M represents independently a metal cation M selected from the group comprising Al³⁺, Ca²⁺, Cu⁺, Cu²⁺, Cr³⁺, Fe²⁺, Fe³⁺, Ga³⁺, Mg²⁺, Mn²⁺, Mn³⁺, Mn⁴⁺, Ti³⁺, Ti⁴⁺, V³⁺, V⁴⁺, Zn²⁺, Zn³⁺, Zr⁴⁺, Ln³⁺ in which Ln is a rare earth or deep transition element;
m is 1, 2, 3, 4, 5, 6, 7, 8, 9, 10, 11 or 12;
k is 0, 1, 2, 3 or 4;
l is 0, 1, 2, 3, 4, 5, 6, 7, 8, 9, 10, 11, 12, 13, 14, 15, 16, 17 or 18;
p is 1, 2, 3, 4, 5 or 6;
X is an anion selected from the group comprising OH⁻, Cl⁻, F⁻, I⁻, Br⁻, SO₄²⁻, NO₃⁻, ClO₄⁻, PF₆⁻, BF₄⁻, R—(COO)ₙ⁻ where R is as defined below, R¹—(COO)ₙ⁻, R¹—(SO₃)ₙ⁻, R¹—(PO₃)ₙ⁻, where R¹ is a hydrogen, a linear or branched, optionally substituted, C₁-C₁₂ alkyl, an aryl, where n is an integer from 1 to 4;
L is a spacer ligand comprising a radical R having q

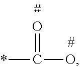

carboxylate groups where
q is 1, 2, 3, 4, 5 or 6; * denotes the point of attachment of the carboxylate to the radical R;
# denotes the possible points of attachment of the carboxylate to the metal ion;
R represents:
(i) a C₁₋₁₂alkyl, C₂₋₁₂alkenyl or C₂₋₁₂alkynyl radical;
(ii) a fused or unfused, mono- or polycyclic aryl radical comprising 6 to 50 carbon atoms;
(iii) a fused or unfused, mono- or polycyclic heteroaryl comprising 1 to 50 carbon atoms;
(iv) an organic radical comprising a metallic element selected from the group comprising ferrocene, porphyrin, phthalocyanine;
the radical R being optionally substituted with one or more groups R², selected independently from the group comprising C₁₋₁₀alkyl; C₂₋₁₀alkenyl; C₂₋₁₀alkynyl; C₃₋₁₀cycloalkyl; C₁₋₁₀heteroalkyl; C₁₋₁₀haloalkyl; C₆₋₁₀aryl; C₃₋₂₀heterocyclic; C₁₋₁₀alkylC₆₋₁₀aryl; C₁₋₁₀alkylC₃₋₁₀heteroaryl; F; Cl; Br; I; —NO₂; —CN; —CF₃; —CH₂CF₃; —OH; —CH₂OH; —CH₂CH₂OH; —NH₂; —CH₂NH₂; —NHCHO; —COOH; —CONH₂; —SO₃H; —CH₂SO₂CH₃; —PO₃H₂; or a function -GR^{G1} in which G is —O—, —S—, —NR^{G2}-, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, where each occurrence of R^{G2} is, independently of the other occurrences of R^{G2}, a hydrogen atom; or a C₁₋₁₂alkyl, C₁₋₁₂heteroalkyl, C₁₋₁₂alkenyl or C₂₋₁₀alkynyl function, linear, branched or cyclic, optionally substituted; or a C₆₋₁₀aryl, C₃₋₁₀heteroaryl, C₅₋₁₀heterocyclic, C₁₋₁₀alkylC₆₋₁₀aryl or C₁₋₁₀alkylC₃₋₁₀heteroaryl group in which the aryl, heteroaryl or heterocyclic radical is optionally substituted; or else, when G represents —NR^{G2}-, R^{G1} and R^{G2}, together with the nitrogen atom to which they are bound, form a heterocycle or a heteroaryl, optionally substituted,

can be used as catalyst for reducing nitrogen oxide.

The inventors in fact discovered, unexpectedly, that the MOF solid defined above, used as catalyst, overcomes all the aforementioned problems and drawbacks of the prior art.

A very large part of the research activities of the present inventors has been and is focused on investigation of catalysts for pollution control. Since the beginning of the 1990s, they have conducted research in the area of removal of NOx: they have investigated several families of catalysts and methods: zeolites, supported precious metals, metal oxides, SCR, NOx-trap, etc., and are continuing to analyze them by a methodology of operative in-situ infrared (IR) spectroscopy notably described in the documents T. Lesage et al., Phys. Chem. Chem. Phys. 5 (2003) 4435 [64]; M. Daturi et al., Phys. Chem. Chem. Phys., 3 (2001) 252 [65] and P. Bazin et al., Stud. Surf. Sci. Catal., 171 (2007) 97 [66].

Owing to this long experience, to advanced techniques for characterization and to an extensive and enduring partnership with several manufacturers of catalytic materials and car makers, the inventors have acquired skills in the area of the removal of NOx that are recognized throughout the world. During the last 15 years their basic and applied research has led to the description of various catalytic mechanisms and to the filing of several patents, generating a culture of understanding of catalysis and of the manner of operation of catalysts which has resulted in a theoretical definition of the catalyst par excellence, capable of attaining the limits imposed by thermodynamics and never achieved previously.

Now, they have found that this new class of materials, the MOFs defined above and hereunder and used in the present invention, has made it possible, completely unexpectedly, to finally make this conceptual approach concrete and finally find what many scientific teams throughout the world have been seeking ardently for the last two decades: a catalyst capable of decomposing, surprisingly, nitrogen oxides at low temperature, even without using reducing agents, which are expensive and polluting. The culmination of the present inventors' research, resulting in the present invention, beyond the obvious implications for society and the environment, constitutes a true consecration as undisputed greater experts (leaders) in catalysis for pollution control. Moreover, the prospects for applications of the present invention in separation in DeNOx catalysis based on these MOFs constitutes a very important innovative direction in terms of applications in an area of public interest for these materials. This subject area, very rich in applications, is of fundamental interest for the present and for the future.

Thus, the present invention relates to the use of porous metal carboxylates possessing unsaturated reducible metal sites M as defined above, in order to transform the toxic species nitrogen oxides into nontoxic gases such as N₂ and O₂. As stated above, the phases in question are porous carboxylates of iron(III), and/or of other transition elements, for example based on trimers of octahedra that possess a large amount of unsaturated metal sites.

The present invention relates more generally to the use of porous inorganic-organic hybrid solids based on elements that can be reduced for catalyzing the conversion of nitrogen oxides.

As stated above, “catalyst for reduction of nitrogen oxide(s)” means, in the present invention, a redox catalyst causing the chemical reduction of nitrogen oxide(s)—in other words a catalyst for reductive catalytic decomposition of nitrogen oxide(s), i.e. for decomposition of nitrogen oxide(s) by chemical transformation.

Nitrogen oxide means, in the present invention, nitric oxide (NO) and nitrogen dioxide (NO₂), collectively designated NOx, as well as nitrous oxide (N₂O), dinitrogen trioxide (N₂O₃) and dinitrogen tetroxide (N₂O₄). It can be one of these gases or a mixture thereof. The nitrogen oxide as defined in the present invention can be alone or combined with other gases, for example those of the atmosphere or from any gaseous or liquid effluent coming from buildings in which nitrogen oxide can be generated.

“Reducible ion of a transition metal M^{z+}” means an ion capable of being reduced, i.e. of gaining one or more electron(s). This ion capable of being reduced is also called in the present invention “reducible metal center” or “activated metal center” or “reducible ion”. Means for obtaining said reducible ion are described hereunder.

In the MOF solid of the present invention, the term “substituted” denotes for example replacement of a hydrogen radical in a given structure with a radical R² as defined above. When more than one position can be substituted, the substituents can be the same or different in each position.

“Spacer ligand” means, in the sense of the present invention, a ligand (including for example neutral species and ions) coordinated with at least two metals, participating in increasing the distance between these metals and the formation of empty spaces or pores. The spacer ligand can comprise 1 to 6 carboxylate groups, as defined above, which can be monodentate or bidentate, i.e. can comprise one or two points of attachment to the metal. The points of attachment to the metal are represented by the symbol # in the formulas. When the structure of a function A has two points of attachment indicated by the symbol “#”, this signifies that the coordination with the metal can be effected by one or other or both points of attachment.

“Alkyl” means, in the sense of the present invention, a linear, branched or cyclic, saturated, optionally substituted, carbon-containing radical comprising 1 to 12 carbon atoms, for example 1 to 10 carbon atoms, for example 1 to 8 carbon atoms, for example 1 to 6 carbon atoms.

“Alkene” means, in the sense of the present invention, an alkyl radical, as defined above, having at least one carbon-carbon double bond.

“Alkenyl” means, in the sense of the present invention, an unsaturated linear, branched or cyclic, optionally substituted, carbon-containing radical containing at least one carbon-carbon double bond, comprising 2 to 12 carbon atoms, for example 2 to 10 carbon atoms, for example 2 to 8 carbon atoms, for example 2 to 6 carbon atoms.

“Alkyne” means, in the sense of the present invention, an alkyl radical, as defined above, having at least one carbon-carbon triple bond.

“Alkynyl” means, in the sense of the present invention, an unsaturated linear, branched or cyclic, optionally substituted, carbon-containing radical containing at least one carbon-carbon triple bond, comprising 2 to 12 carbon atoms, for example 2 to 10 carbon atoms, for example 2 to 8 carbon atoms, for example 2 to 6 carbon atoms.

“Aryl” means, in the sense of the present invention, an aromatic system comprising at least one ring complying with Hückel's rule for aromaticity. Said aryl is optionally substituted and can comprise from 6 to 50 carbon atoms, for example 6 to 20 carbon atoms, for example 6 to 10 carbon atoms.

“Heteroaryl” means, in the sense of the present invention, a system comprising at least one aromatic ring with 5 to 50 ring members, among which at least one group of the aromatic ring is a heteroatom, notably selected from the group comprising sulfur, oxygen, nitrogen, boron. Said heteroaryl is optionally substituted and can comprise from 1 to 50 carbon atoms, preferably 1 to 20 carbon atoms, preferably 3 to 10 carbon atoms.

“Cycloalkyl” means, in the sense of the present invention, a cyclic, saturated or unsaturated, optionally substituted, carbon-containing radical which can comprise 3 to 20 carbon atoms, preferably 3 to 10 carbon atoms.

“Haloalkyl” means, in the sense of the present invention, an alkyl radical as defined above, said alkyl system comprising at least one halogen.

“Heteroalkyl” means, in the sense of the present invention, an alkyl radical as defined above, said alkyl system comprising at least one heteroatom, notably selected from the group comprising sulfur, oxygen, nitrogen, boron. For example, it can be an alkyl radical bound covalently to the rest of the molecule by a heteroatom selected from sulfur, oxygen, nitrogen or boron. Thus, a heteroalkyl can be represented by the group -GR^{G1} in which R^{G1} represents an alkyl radical as defined above, and G represents —O—, —S—, —NR^{G2}— or —BR^{G2}—, in which R^{G2} represents H; a linear, branched or cyclic alkyl, alkenyl or alkynyl radical; a C₆₋₁₀ aryl group; or a C₁₋₆ acyl radical (“acyl” denoting a radical —C(═O)R where R represents an alkyl radical as defined above). For example, G can represent —O—, —S—, or —NR^{G2}—, in which R^{G2} is as defined above. It is understood that when G represents —NR^{G2}—, R^{G2} can also represent a protective group. A person skilled in the art can refer notably to the work of P. G. M. Wuts & T. W. Greene, “Greene's Protective Groups in Organic Synthesis”, fourth edition, 2007, Publ. John Wiley & Son [97], for choosing appropriate protective groups. In the present invention, the term “C₁₋₆heteroalkyl” can represent a group -GR^{G1} as defined above, in which R^{G1} represents a linear, branched or cyclic C₁₋₆ alkyl, C₂₋₆ alkenyl or C₂₋₆ alkynyl radical. For example, “C₁₋₆heteroalkyl” can represent a group -GR^{G1} as defined above, in which R^{G1} represents a linear, branched or cyclic C₁₋₆ alkyl radical.

“Heterocycle” means, in the sense of the present invention, a carbon-containing cyclic radical comprising at least one heteroatom, saturated or unsaturated, optionally substituted, and which can comprise 2 to 20 carbon atoms, preferably 5 to 20 carbon atoms, preferably 5 to 10 carbon atoms. The heteroatom can be selected for example from the group comprising sulfur, oxygen, nitrogen, boron.

“Alkoxy”, “aryloxy”, “heteroalkoxy” and “heteroaryloxy” mean, in the sense of the present invention, respectively an alkyl, aryl, heteroalkyl and heteroaryl radical bound to an oxygen atom.

“Alkylthio”, “arylthio”, “heteroalkylthio” and “heteroarylthio” mean, in the sense of the present invention, respectively an alkyl, aryl, heteroalkyl and heteroaryl radical bound to a sulfur atom.

The particular crystalline structure of the MOF solids according to the invention endows these materials with specific properties.

In the MOF solids of the invention, M can advantageously be Cu⁺, Cu²⁺, Fe²⁺, Fe³⁺, Mn²⁺, Mn³⁺, Mn⁴⁺, Ti³⁺, Ti⁴⁺, V³⁺, V⁴⁺, Zn²⁺, Zn³⁺, Ln³⁺ in which Ln is a rare earth or deep transition element. M can also be a mixture of these metals. According to the invention, M can advantageously be selected from the group comprising Al, Fe, Ca, Cr, Cu, Ga, Ln when Ln is an element belonging to the rare earths or a deep transition element, Mg, Mn, Ti, V, Zn and Zr. M can also be a mixture of these metals. M is advantageously Fe, Mn, Ti, V, Zn and Cu. M can also be a mixture of these metals. For example, iron is a biocompatible metal which does not pose any major problem for the environment.

As stated above, M can be a metal ion M^{z+} in which z is from 2 to 4. M may or may not be a transition metal. When M is a mixture of metals, z can have an identical or different value for each metal.

In one embodiment of the invention, the solids of the invention can comprise a three-dimensional succession of units of formula (I) in which M can represent a single type of ion M^{z+}, for example Fe or one of the other metals mentioned above, in which z can be identical or different, for example 2, 3 or a mixture of 2 and 3.

In another embodiment of the invention, the solids of the invention can comprise a three-dimensional succession of units of formula (I) in which M can represent a mixture of different ions M^{z+}, for example Fe and Ti, for example Fe and Cu, for example Fe and Zn, etc., in which for each metal ion M^{z+}, z can be identical or different, for example 2, 3, 4 or a mixture of 2, 3 and 4.

In a particular embodiment, M^{z+} represents trivalent octahedral Fe with z equal to 3. In this embodiment, Fe has a coordination number of 6.

“Coordination number” means the number of bonds that a cation forms with anions.

The metal ions can be isolated or can be grouped in metal “clusters”. The MOF solids according to the invention can for example be constructed from chains of octahedra or trimers of octahedra.

“Metal cluster” means, in the sense of the present invention, an ensemble of atoms containing at least two metal ions bound by ion-covalent bonds, either directly by anions, for example O₂⁻, OH⁻, Cl⁻, etc., or by the organic ligand.

Moreover, the MOF solids according to the invention can be in different forms or “phases” taking into account the various possibilities of organization and of connections of the ligands to the metal ion or to the metal group.

“Phase” means, in the sense of the present invention, a hybrid composition comprising at least one metal and at least one organic ligand possessing a defined crystalline structure.

The crystalline spatial organization of the solids of the present invention accounts for the particular characteristics and properties of these materials. It notably governs the pore size, which has an influence on the specific surface of the materials and on the diffusion of gas molecules within them. It also governs the density of the materials, which is relatively low, the proportion of metal in these materials, the stability of the materials, the rigidity and flexibility of the structures, etc.

Moreover, the pore size can be adjusted by choosing appropriate ligands L.

The ligand L of the unit of formula (I) of the present invention can be for example a polycarboxylate, for example a di-, tri-, tetra- or hexa-carboxylate. It can be selected for example from the group comprising: C₂H₂(CO₂⁻)₂, for example fumarate; C₂H₄(CO₂⁻)₂, for example succinate; C₃H₆(CO₂⁻)₂, for example glutarate; C₄H₄(CO₂⁻)₂, for example muconate; C₄H₈ (CO₂⁻)₂, for example adipate; C₇H₁₄ (CO₂⁻)₂, for example azelate; C₅H₃S(CO₂⁻)₂, for example 2,5-thiophenedicarboxylate; C₆H₄(CO₂⁻)₂, for example terephthalate; C₆H₂N₂(CO₂⁻)₂, for example 2,5-pyrazine dicarboxylate: C₁₀H₆ (CO₂⁻)₂, for example 2,5-naphthalene-2,6-dicarboxylate; C₁₂H₈(CO₂⁻)₂, for example biphenyl-4,4′-dicarboxylate; C₁₂H₈N₂(CO₂⁻)₂, for example azobenzenedicarboxylate, dichloroazobenzenedicarboxylate, azobenzenetetracarboxylate, dihydroxoazobenzenedicarboxylate; C₆H₃ (CO₂⁻)₃, for example benzene-1,2,4-tricarboxylate; C₆H₃(CO₂⁻)₃, for example benzene-1,3,5-tricarboxylate; C₂₄H₁₅(CO₂⁻)₃, for example benzene-1,3,5-tribenzoate, 1,3,5-tris[4′-carboxy(1,1′-biphenyl-4-yl)benzene; C₆H₂(CO₂⁻)₄, for example benzene-1,2,4,5-tetracarboxylate; C₁₀H₄(CO₂⁻)₄, for example naphthalene-2,3,6,7-tetracarboxylate; C₁₀H₄ (CO₂⁻)₄, for example naphthalene-1,4,5,8-tetracarboxylate; C₁₂H₆(CO₂⁻)₄, for example biphenyl-3,5,3′,5′-tetracarboxylate; and their modified analogs selected for example from the group comprising 2-aminoterephthalate, 2-nitroterephthalate, 2-methylterephthalate, 2-chloroterephthalate, 2-bromoterephthalate, 2,5-dihydroxoterephthalate, tetrafluoroterephthalate, 2,5-dicarboxyterephthalate, tetramethylterephthalate, dimethyl-4,4′-biphenyldicarboxylate, tetramethyl-4,4′-biphenyldicarboxylate, dicarboxy-4,4′-biphenyldicarboxylate, 2,5-pyrazyne dicarboxylate. The ligand can also be 2,5-diperfluoroterephthalate, azobenzene-4,4′-dicarboxylate, 3,3′-dichloro-azobenzene-4,4′-dicarboxylate, 3,3′-dihydroxo-azobenzene-4,4′-dicarboxylate, 3,3′-diperfluoro azobenzene-4,4′-dicarboxylate, 3,5,3′,5′-azobenzene tetracarboxylate, 2,5-dimethyl terephthalate, perfluorosuccinate, perfluoromuconate, perfluoroglutarate, 3,5,3′,5′-perfluoro-4,4′-azobenzene dicarboxylate, 3,3′-diperfluoro-azobenzene-4,4′-dicarboxylate.

Furthermore, the ligand can have biological activity. It can be one of the ligands mentioned above, displaying biological activity, for example a ligand selected from C₇H₁₄(CO₂⁻)₂ (azelate); an aminosalicylate, for example carboxyl, amino and hydroxyl groups; a porphyrin with carboxylate functions, amino acids, for example the natural or modified amino acids known by a person skilled in the art, for example Lys, Arg, Asp, Cys, Glu, Gln, etc., with amino, carboxylate, amide and/or imine groups; an azobenzene with carboxylate groups; dibenzofuran-4,6-dicarboxylate, dipicolinate; glutamate, fumarate, succinate, suberate, adipate, nicotinate, nicotinamide, purines, pyrimidines.

The ligand can be either in its acid form or ester of carboxylic acid, or in the form of a metal salt, for example of sodium or of potassium, of carboxylic acid.

The ligand can be either in its acid form or ester of carboxylic acid, or in the form of a metal salt, for example of sodium or of potassium, of carboxylic acid.

The anion X of the unit of formula (I) of the present invention can for example be selected from the group comprising OH⁻, Cl⁻, F⁻, R— (COO)ₙ⁻, PF₆⁻, ClO₄⁻, with R and n as defined above.

The MOF solid according to the invention can comprise for example a percentage by weight of M in the dry phase from 5 to 50%, for example preferably from 18 to 31%.

The percentage by weight (wt. %) is a unit of measurement used in chemistry and in metallurgy to denote the composition of a mixture or of an alloy, i.e. the proportions of each component in the mixture. This unit is used in the present text.

1 wt. % of a component=1 g of the component per 100 g of mixture or 1 kg of said component per 100 kg of mixture.

The MOF solids of the present invention notably have the advantage of possessing thermal stability up to a temperature of 350° C. More particularly, these solids have thermal stability of 120° C. and 350° C.

According to the invention, the MOF solid can have for example a pore size from 0.4 to 6 nm, preferably from 0.5 to 5.2 nm, and more preferably 0.5 to 3.4 nm.

According to the invention, the MOF solid can have a gas loading capacity from 0.5 to 50 mmol of gas per gram of dry solid.

According to the invention, the MOF solid can have for example a specific surface (BET) from 5 to 6000 m²/g, preferably from 5 to 4500 m²/g.

According to the invention, the MOF solid can have for example a pore volume from 0 to 4 cm³/g, preferably from 0.05 to 2 cm³/g. In the context of the invention, the pore volume signifies the volume accessible by the gas molecules.

In the MOF solids of the invention, at least a proportion of the Lewis-base gas or gases is coordinated with M. According to the invention, for example at least 1 to 5 mmol of gas per gram of dry solid is coordinated with M. The portion of the gas or gases that is not coordinated with M can advantageously fill the free space in the pores.

The MOF solid of the present invention can be in the form of a robust structure, which has a rigid framework and only contracts very slightly when the pores are emptied of their contents, which can be, for example, solvent, noncoordinated carboxylic acid, etc. It can also be in the form of a flexible structure, which can swell and deflate, causing the opening of the pores to vary as a function of the nature of the molecules adsorbed, which can be, for example, solvents and/or gases.

“Rigid structure” means, in the sense of the present invention, structures that swell or shrink only very slightly, i.e. with an amplitude up to 10%. In particular, the MOF solid according to the invention can have a rigid structure that swells or shrinks with an amplitude in the range from 0 to 10%. The rigid structures can for example be constructed on the basis of chains or trimers of octahedra. According to one embodiment of the invention, the MOF solid of rigid structure can have a percentage by weight of M in the dry phase from 5 to 50%, preferably from 18 to 31%. Advantageously, M will represent iron here. The MOF solid of rigid structure according to the invention can have a pore size from 0.4 to 6 nm, in particular from 0.5 to 5.2 nm, more particularly from 0.5 to 3.4 nm. The MOF solid of rigid structure according to the invention can have a pore volume from 0.5 to 4 cm³/g, in particular from 0.05 to 2 cm³/g.

“Flexible structure” means, in the sense of the present invention, structures that swell or shrink with a large amplitude, notably with an amplitude greater than 10%, preferably greater than 50%. The flexible structures can for example be constructed on the basis of chains or trimers of octahedra. In particular, the MOF material according to the invention can have a flexible structure that swells or shrinks with an amplitude from 10% to 300%, for example from 50 to 300%.

In a particular embodiment of the invention, the MOF solid of flexible structure can have a percentage by weight of M in the dry phase from 5 to 40%, preferably from 18 to 31%. Advantageously, M will represent iron here.

For example, in the context of the invention, the MOF solid of flexible structure can have a pore size from 0.4 to 6 nm, in particular from 0.5 to 5.2 nm, and more particularly from 0.5 to 1.6 nm.

For example, the MOF solid of flexible structure according to the invention can have a pore volume from 0 to 3 cm³/g, in particular from 0 to 2 cm³/g.

Moreover, the inventors have demonstrated experimentally that the amplitude of the flexibility depends on the nature of the ligand and of the solvent used, as described in the “Examples” section below.

According to the invention, whatever the structure, the MOF solid possesses for example an amount of unsaturated metal sites M from 0.5 to 7 mmol/g of solid, for example from 1 to 3 mmol/g, in particular from 1.3 to 3.65 mmol/g of solid.

Various MOF materials have been developed by the inventors at the Institut Lavoisier of Versailles designated “MIL” (for “Material Institut Lavoisier”). The designation “MIL” of these structures is followed by an arbitrary number n given by the inventors for identifying the various solids.

In the context of the present invention, the inventors have demonstrated that MOF solids can comprise a three-dimensional succession of units corresponding to formula (I).

In a particular embodiment of the invention, the MOF solids can comprise a three-dimensional succession of iron(III) carboxylates corresponding to formula (I). These iron(III) carboxylates can be selected from the group comprising MIL-88, MIL-89, MIL-96, MIL-100, MIL-101, MIL-102, MIL-126 and MIL-127, for example among those shown in Table B below and in the “Examples” section below.

In particular, the MOF solids can comprise a three-dimensional succession of units corresponding to formula (I), selected from the group comprising:

Fe₃OX[O₂C—C₂H₂—CO₂]₃ of flexible structure

Fe₃OX[O₂C—C₆H₄—CO₂]₃ of flexible structure

Fe₃OX[O₂C—C₁₀H₆—CO₂]₃ of flexible structure

Fe₃OX[O₂C—C₁₂H₈—CO₂]₃ of flexible structure

Fe₃OX [O₂C—C₄H₄—CO₂]₃ of flexible structure

Fe(OH) [O₂C—C₄H₄—CO₂] of flexible structure

Fe₁₂O(OH)₁₈(H₂O)₃ [C₆H₃—(CO₂)₃]₆ of rigid structure

Fe₃OX [C₆H₃—(CO₂)₃]₂ of rigid structure

Fe₃OX [O₂C—C₆H₄—CO₂]₃ of rigid structure

Fe₆O₂X₂ [C₁₀H₂—(CO₂)₄]₃ of rigid structure

Fe₆O₂X₂ [C₁₄H₈N₂—(CO₂)₄]₃ of rigid structure in which X is as defined above.

Examples of MOFs usable in the present invention are given in Table B below:

TABLE B

| | | Metal | |
|---|---|---|---|
| | | Sites | |
| Phase | Composition | (mmol · g⁻¹) | Framework |
| MIL-88A | Fe₃OX[O₂C—C₂H₂—CO₂]₃•nH₂O | 3.7 | flexible |
| (fumarate) | | | |
| MIL-88B | Fe₃OX[O₂C—C₆H₄—CO₂]₃•nH₂O | 2.9 | flexible |
| (terephthalate)⁹* | | | |
| MIL-88C | Fe₃OX[O₂C—C₁₀H₆—CO₂]₃•nH₂O | 2.4 | flexible |
| (2,6 napthalene | | | |
| dicarboxylate)⁹ | | | |
| MIL-88D | Fe₃OX[O₂C—C₁₂H₈—CO₂]₃•nH₂O | 2.2 | flexible |
| (4,4′ biphenyl | | | |
| dicarboxylate)⁹* | | | |
| MIL-89 | Fe₃OX[O₂C—C₄H₄—CO₂]₃•nH₂O | 3.2 | flexible |
| (trans, trans | | | |
| Muconate)⁸ | | | |
| MIL-88B-Cl | Fe₃OX[O₂C—C₆H₃(Cl)—CO₂]₃•nH₂O | 2.5 | flexible |
| (2-chloro | | | |
| terephthalate) | | | |
| MIL-88B-Br | Fe₃OX[O₂C—C₆H₃(Br)—CO₂]₃•nH₂O | 2.14 | flexible |
| (2-bromo | | | |
| terephthalate) | | | |
| MIL-88B-NO₂ | Fe₃OX[O₂C—C₆H₃(NO₂)—CO₂]₃•nH₂O | 2.41 | flexible |
| (2-nitro | | | |
| terephthalate) | | | |
| MIL-88B-NH₂ | Fe₃OX[O₂C—C₆H₃(NH₂)—CO₂]₃•nH₂O | 2.70 | flexible |
| (2-amino | | | |
| terephthalate) | | | |
| MIL-88B-2OH | Fe₃OX[O₂C—C₆H₂(OH)₂—CO₂]₃•nH₂O | 2.53 | flexible |
| (2,5-dihydroxo | | | |
| terephthalate) | | | |
| MIL-88B-4F | Fe₃OX[O₂C—C₆F₄—CO₂]₃•nH₂O | 2.3 | flexible |
| (tetrafluoro | | | |
| terephthalate) | | | |
| MIL-88B-4CH₃ | Fe₃OX[O₂C—C₆(CH₃)₄—CO₂]₃•nH₂O | 2.3 | flexible |
| (tetramethyl | | | |
| terephthalate) | | | |
| MIL-88F | Fe₃O[C₄H₂S—(CO₂)₂]₃•X•nH₂O | 2.75 | flexible |
| (2,5 thiophene | | | |
| dicarboxylate) | | | |
| MIL-88G | Fe₃OX[C₁₂H₈N₂—(CO₂)₂]₃•nH₂O | 1.99 | flexible |
| (4,4′ azobenzene | | | |
| dicarboxylate) | | | |
| MIL-88G-2Cl | Fe₃OX[C₁₂H₆Cl₂N₂—(CO₂)₂]₃•nH₂O | 1.86 | flexible |
| (3,3′-dichloro4,4′- | | | |
| azobenzenedicarboxylate) | | | |
| MIL-96¹⁰* | Fe₁₂O(OH)₁₈(H₂O)₃[C₆H₃—(CO₂)₃]₆•nH₂O | 1.3 | rigid |
| (trimesate) | | | |
| MIL-100 | Fe₃OX[C₆H₃—(CO₂)₃]₂•nH₂O | 3.65 | rigid |
| (trimesate)² | | | |
| MIL-101 | Fe₃OX[O₂C—C₆H₄—CO₂]₃•nH₂O | 2.9 | rigid |
| (terephthalate)¹¹* | | | |
| MIL-102 | Fe₆O₂X₂[C₁₀H₂—(CO₂)₄]₃•nH₂O | 3.1 | rigid |
| (1,4,5,8 | | | |
| Naphthalene | | | |
| Tetracarboxylate)⁷* | | | |
| MIL-126 | Fe₃OX[O₂C—C₁₂H₈—CO₂]₃•nH₂O | 2.2 | flexible |
| (4,4′ biphenyl | | | |
| dicarboxylate) | | | |
| MIL-127 | Fe₆O₂X₂[C₁₂H₆N₂—(CO₂)₄]₃•nH₂O | 2.72 | rigid |
| (3,3′-5,5′- | | | |
| azobenzenetetracarboxylate) | | | |
| MIL-53 | Fe(OH)[C6H4—(CO2)2]•nH2O | — | flexible |
| (terephthalate) | | | |

** calculations presented, done with X = F.

In these examples, X can be as defined above in the definition of the MOF solid comprising the units of formula (I) used in the present invention. It can be for example OH⁻, Cl⁻, F⁻, I⁻, Br⁻, SO₄²⁻, NO₃⁻, etc.

Synthesis protocols of these various compounds are described for example in the following documents:
Syntheses of MIL-88A and MIL-89 described for example in Serre C et al., Angew. Chem. Int. Ed. 2004, 43, 6286: A new route to the synthesis of trivalent transition metals porous carboxylates with trimeric SBU [67].
Syntheses of MIL-88B, MIL-88C, MIL-88D described for example in Surblé S et al., Chem. Comm. 2006 284: A new isoreticular class of Metal-Organic-Frameworks with MIL-88 topology [68].
Synthesis of MIL-96 described for example in Loiseau T et al., J. Am. Chem. Soc. 2006, 128, 10223: MIL-96, a Porous Aluminum Trimesate 3D Structure Constructed from a Hexagonal Network of 18-Membered Rings and i3-Oxo-Centered Trinuclear Units [69].
Synthesis of MIL-100 described for example in Horcajada P et al., Chem Comm, 2007, 2820: Synthesis and catalytic properties of MIL-100(Fe), an iron(III) carboxylate with large pore [70].
Synthesis of MIL-101 described for example in Gérard Férey, et al., Science 2005 309, 2040: A Chromium Terephthalate-Based Solid with Unusually Large Pore Volumes and Surface Area [71].
Synthesis of MIL-102 described for example in Surblé S et al., J. Am. Chem. Soc. 128 (2006), 46, 14890. MIL-102: A Chromium Carboxylate Metal Organic Framework with Gas Sorption Analysis [72].
Syntheses of MIL-53 described for example in T. R. Whitfield et al., Solid State Sci., 2005, 7, 1096-1103: Metal-organic frameworks based on iron oxide octahedral chains connected by benzenedicarboxylate dianions [94].
Synthesis of HKUST-1 (Cu₃[(CO₂)₃C₆H₃]₂ (H₂O)₃) described for example in S. S.-Y. Chui et al., Science, 283, 1148-1150: A chemically functionalizable material [Cu₃(TMA)₂(H₂O)₃]ₙ [96].

These documents describe the synthesis of isostructural solids sometimes with other cations (Cr, V, Al). In this case, in the protocols for producing the MOFs described above, the metal is replaced by Fe or another metal according to the definition of M given above in formula (I). Other examples of protocols for manufacture of MOFs presented in the above table are given in the “Examples” section below.

Moreover, starting from one and the same carboxylic acid ligand L and the same iron bases (trimers), the inventors were able to obtain MOF materials of the same general formula (I) but with different structures. This applies for example to the solids MIL-88B and MIL-101. In fact, the solids MIL-88B and MIL-101 differ in the manner of connection of the ligands to the octahedral trimers: in the solid MIL-101, the ligands L assemble in the form of rigid tetrahedra, whereas in the solid MIL-88B, they form triangular bipyramids, making spacing possible between the trimers.

These various materials are presented in the “Examples” section below. The manner of assembly of these ligands can be controlled during synthesis for example by adjusting the pH. For example, the solid MIL-88 is obtained in a less acid medium than the solid MIL-101 as described in the “Examples” section below.

The MOF solids as defined in the present invention can be prepared by any method known by a person skilled in the art. They can be prepared for example by a solvothermal or hydrothermal route, by microwave or ultrasonic synthesis or by mechanical grinding.

It can be for example a method comprising the following reaction step:

(i) mix, in a polar solvent:
at least one solution comprising at least one inorganic metallic precursor in the form of a metal M, a metal salt of M or a coordination complex comprising a metal ion of M, M being as defined above,
at least one ligand L′ comprising a radical R having q groups *—C(═O)—R³, where
   q and R are as defined above,
   * denotes the point of attachment of the group to the radical R,
   R³ is selected from the group comprising an OH, an OY, where Y is an alkaline cation, a halogen, a radical —OR⁴, —O—C(═O)R⁴, —NR⁴R⁴, where R⁴ and Ru are C₁₋₁₂ alkyl radicals,
      to obtain an MOF material.

Whatever method is used, according to the invention, the precursors of M for the manufacture of MOF solids can be:
metal salts, for example nitrates, chlorides, sulfates, acetates, oxalates of M,
alkoxides,
oxo or hydroxo polymetallic clusters,
organometallic complexes
a metal powder
or any other suitable precursor.

Whatever method is used, according to the invention, the preparation time can vary for example from 1 minute up to several weeks, ideally between 1 minute and 72 hours.

Whatever method is used, according to the invention, the preparation temperature can be for example from 0° C. to 220° C., ideally from 20° C. to 180° C.

The preparation of MOF materials can preferably be carried out in the presence of energy, which can be supplied for example by heating, for example hydrothermal or solvothermal conditions, but also by microwaves, by ultrasound, by grinding, by a method involving a supercritical fluid, etc. The corresponding protocols are those known by a person skilled in the art. Nonlimiting examples of protocols applicable for hydrothermal or solvothermal conditions are described for example in K. Byrapsa, et al., “Handbook of hydrothermal technology”, Noyes Publications, Parkridge, N.J. USA, William Andrew Publishing, LLC, Norwich N.Y. USA, 2001 [73]. For synthesis using microwaves, nonlimiting examples of suitable protocols are described for example in G. Tompsett et al. ChemPhysChem. 2006, 7, 296 [74]; S-E. Park et al., Catal. Survey Asia 2004, 8, 91 [75]; C. S. Cundy, Collect. Czech. Chem. Commun. 1998, 63, 1699 [76]; S. H. Jhung, J.-H. Lee, J.-S. Chang, Bull. Kor. Chem. Soc. 2005, 26, 880 [77]. For the conditions when using a roll-type grinding mill, reference may be made for example to the works by Pichon, Cryst. Eng. Comm. 8, 2006, 211-214 [78].; D. Braga, Angew. Chem. Int. Ed. 45, 2006, 142-246 [79].; D. Braga, Dalton Trans., 2006, 1249-1263 [80]. Hydrothermal or solvothermal conditions, in which the reaction temperatures can vary between 0 and 220° C., are generally applied in glass (or plastic) vessels when the temperature is below the boiling point of the solvent. When the temperature is higher or when the reaction is carried out in the presence of fluorine, Teflon containers inserted in metal bombs are used [73].

The solvents used are generally polar. Notably, the following solvents can be used: water, alcohols, dimethylformamide, dimethylsulfoxide, acetonitrile, tetrahydrofuran, diethylformamide, chloroform, cyclohexane, acetone, cyanobenzene, dichloromethane, nitrobenzene, ethylene glycol, dimethylacetamide or mixtures of these solvents.

One or more co-solvents can also be added in any step of the synthesis for better dissolution of the compounds of the mixture. These can notably be monocarboxylic acids, such as acetic acid, formic acid, benzoic acid, etc.

One or more additives can also be added during the synthesis in order to modulate the pH of the mixture. These additives can be selected for example from mineral or organic acids or mineral or organic bases. For example, the additive can be selected from the group comprising: HF, HCl, HNO₃, H₂SO₄, NaOH, KOH, lutidine, ethylamine, methylamine, ammonia, urea, EDTA, tripropylamine, pyridine.

Preferably, reaction step (i) can be carried out according to at least one of the following reaction conditions:
with a reaction temperature from 0° C. to 220° C., preferably from 50 to 150° C.;
with a stirring speed from 0 to 1000 rpm (revolutions per minute), preferably from 0 to 500 rpm;
with a reaction time from 1 minute to 144 hours, preferably from 1 minute to 15 hours;
with a pH from 0 to 7, preferably from 1 to 5;
with addition of at least one co-solvent to the solvent, to the precursor, to the ligand or to the mixture thereof, said co-solvent being selected from the group comprising acetic acid, formic acid, benzoic acid;
in the presence of a solvent selected from the group comprising water, the alcohols R^{S}—OH where R^{S} is a linear or branched C₁-C₆ alkyl radical, and benzyl alcohol where the alcohol is R^{S}—OH where R^{S} is a linear or branched C₁-C₆ alkyl radical, dimethyl- and diethyl-formamide, dimethylsulfoxide, acetonitrile, tetrahydrofuran, diethyl formamide, chloroform, cyclohexane, acetone, cyanobenzene, dichloromethane, nitrobenzene, ethylene glycol, dimethylacetamide, benzoic alcohol or a mixture of these solvents, whether miscible or not;
in a supercritical medium, for example in supercritical CO₂;
under microwaves and/or under ultrasound;
in conditions of electrochemical electrolysis;
in conditions using a grinding mill, for example a roll mill or ball mill;
in a gas stream.

According to the invention, a surface modifier can be added during or after synthesis of the MOF solids. This surface modifier can be selected from the group comprising polyethylene glycols (PEG), polyvinylpyrrolidones, 2,3-dihydroxobenzoic acid or a mixture thereof. The latter can be grafted or deposited on the surface of the solids, for example adsorbed on the surface or bound by covalent bond, by hydrogen bond, by van der Waals forces, by electrostatic interaction. The surface modifier can also be incorporated by entanglement during manufacture of the MOF solids [81, 82].

Completely surprisingly, the porous MOFs defined above, i.e. based on iron(III) and/or other transition elements possessing unsaturated metal sites, make it possible to catalyze, even at low temperature, i.e. at temperatures below 200° C., the reduction of nitrogen oxides without using a reducing agent, whereas the use of reducing agents is indispensable with the catalysts of the prior art.

Thus, according to the invention, toxic NOx gas molecules interact specifically with the accessible metal sites of the MOF solids, whether or not reduced beforehand, which makes it possible to convert them, at low temperature, i.e. below 200° C., even in the absence of reducing species, and optionally in the presence of oxygen and/or optionally of water, to nontoxic species N₂ and O₂, or less toxic species such as N₂O.

According to the present invention, the use can comprise a step of contacting said MOF solid with the nitrogen oxide to be reduced. It is this contacting that causes the catalysis of oxidation of the nitrogen oxide to nonpolluting gases, for example N₂ and O₂.

For this contacting, the MOF solid can be used directly or can be activated prior to use. It is activated notably when the MOF cannot or cannot sufficiently reduce the nitrogen oxide, notably owing to its oxidation number and/or the amount of active MOF solids and/or the presence of impurities.

According to the present invention, the contacting step can therefore be preceded by a step of activation of the MOF solid, for example by heating under vacuum or under reducing or neutral atmosphere. According to the invention, the step of activation by heating can be carried out at a temperature from 30 to 350° C., for example from 150 to 280° C., preferably from 50 to 250° C. This activation step can be performed for any appropriate duration for obtaining the expected result. The duration depends notably on the actual nature of the material and on the activation temperature. Generally, at the aforementioned temperatures, this activation time can be from 30 to 1440 minutes, for example from 60 to 720 minutes. In practice it is a matter of activating the metal sites, making them accessible so that they reduce the nitrogen oxide, for example to nonpolluting chemical species. Activation can also be carried out under a stream of NOx.

Suitable protocols for activation are for example:
Under primary or secondary vacuum from 13.33 Pa to 13.33×10⁻⁵ Pa (i.e. 10⁻¹ to 10⁻⁶ torr) at constant or variable temperature, from 30 to 400° C., preferably from 50 to 280° C., for a duration from 1 to 100 hours, preferably 2 to 16 hours.
Under a stream of neutral gas, for example helium, nitrogen, argon, etc. at constant or variable temperature, between 30 and 400° C., preferably between 50 and 280° C., for a duration from 1 to 100 hours, preferably 2 to 16 hours.
In NOx mixture at constant or variable temperature, from 30 to 400° C., preferably from 50 to 280° C., for a duration from 1 to 100 hours, preferably 2 to 16 hours.

Activation can be carried out in a controlled manner so as to cause the MOF to interact with the NOx species in order to decompose the latter. This control can be provided by infrared spectroscopy, by monitoring the changes of the spectra of the samples during the activation process, until the spectrum of an activated sample is obtained.

The contacting itself can be passive or active. “Passive” means natural contact of the atmosphere or of the effluent containing the nitrogen oxides with the MOF solid. “Active” means forced or prolonged contacting, notably in a suitable device for example for confining the effluent to be treated.

Contacting can be carried out for a contact time or contacting duration which can be modified depending on the use to which the present invention is put. For example, a contact time of the effluent to be treated of less than 2 minutes is sufficient, for example from 0.03 to 0.72 seconds, for example from 0.1 to 0.50 seconds. This contact time depends notably on the content of nitrogen oxide in the effluent to be treated, the surface area of contacting of the effluent with the MOF solid used for catalysis, the actual nature of the MOF used, the nature of the effluent, the contacting conditions, for example temperature and pressure and the aim pursued by application of the present invention. For example, by measuring the content of nitrogen oxide remaining in an effluent treated by application of the present invention, the time of contacting of the effluent with the catalyst can easily be adjusted, so as to minimize the content of nitrogen oxide in the treated effluent; the objective in treating an effluent being of course to remove the nitrogen oxide.

When applying the present invention, contacting can be performed advantageously in the presence of oxygen and/or water. DeNOx catalysis is in fact improved in these conditions. For example, an amount of oxygen from 0.1% to 20%, for example from 1% to 10%, and/or an amount of water from 0.1% to 10%, for example from 1% to 4%, can be used.

The use of the present invention finds many applications, notably in any method of removal or conversion of nitrogen oxide, whether for experimental, industrial, environmental and/or decontamination purposes. It also finds application in research, in any chemical method of catalysis, for space applications, etc.

In the present text, the terms “medium/media” and “effluent(s)” are used equivalently. According to the invention, the medium in question can be a liquid or gaseous effluent. The effluent can come for example from combustion of hydrocarbons or from oxidation of nitrogen compounds. It can be for example an effluent selected from water, an effluent from a vehicle, from a train, from a boat, for example an exhaust gas, a liquid or gaseous effluent from a factory, a workshop, a laboratory, stored products, cargo, from air vents, notably urban, from air conditioning, from an air purifier, chemical products comprising spills of nitrogen compounds in water and/or in soil, notably fertilizers, drains, discharges, etc.

The nitrogen oxide can be alone or present among other gases, for example among other gases from combustion, for example of hydrocarbons; for example among the gases of the atmosphere, in this case the other gases are notably O₂, N₂ and CO₂; for example, among exhaust gases from a vehicle, a train, a boat, from ducting for aeration and/or ventilation of industrial buildings, parking lots, tunnels, underground transport systems, residential accommodation, laboratories, etc.

The MOF solid used can be in any appropriate form, notably to facilitate its contact with the effluent whose nitrogen oxide must be reduced or removed and to facilitate its use in the proposed application. For example, certain of the methods of manufacture described in the present text and the cited documents make it possible to obtain nanoparticles. These materials form a regular porous network. In general, the MOF solid used in the present invention can be for example in a form selected from nanoparticles, powder, pebbles, granules, pellets, a coating.

According to the invention, when the MOF solid is used in the form of a coating, it can be applied on a flat or non-flat, smooth or non-smooth surface. In order to optimize contact between the effluent and the MOF solid, the surface is preferably not flat and not smooth. The surface can be selected for example from the group comprising a striated surface, a honeycomb, a grid, an organic or mineral foam, a filter, a wall of a building, ducts for ventilation and/or aeration, etc.

The MOF solid can be applied on any type of surface that is suitable for use according to the present invention. It can be for example a surface selected from a surface of paper, glass, ceramic, silicon carbide, cardboard, paper, metal, for example stainless steel, concrete, wood, plastic, etc.

For applying the MOF solid on a surface, according to the present invention, any technique and any suitable material can be used.

For example the MOF can be used alone, i.e. applied on its own on the surface. It is also possible to apply an adhesive coating on the surface before applying the MOF solid. The MOF can also be mixed with a binder, the mixture of MOF and binder then being applied on the surface.

According to the invention, it is possible to use a single MOF or a mixture of MOFs as defined in the present text. Similarly, it is possible to use the single MOF or the mixture of MOFs mixed with other materials, and these other materials can be for example materials that simply support said MOF or said mixture of MOFs or materials which are themselves catalysts of nitrogen oxides such as those described in the prior art, for example above, or, for example, materials that are catalysts of other chemical reactions, for example of decomposition of other toxic gases. Thus, when the “decomposition of nitrogen oxides without using reducing agents other than the MOFs” is mentioned in the present text, this defines the intrinsic activity or capacity of the MOFs according to the present invention, and said activity can be manifested with a material consisting of MOF or of a mixture of MOFs, or of a material comprising an MOF, i.e. an MOF and a material different from an MOF, whether or not this other material has a catalytic activity and whether this activity is identical to or different from that of the MOFs described in the present text.

Advantageously, once manufactured, the MOF solid can be in the form of a colloidal sol that can easily be deposited in the form of a thin, homogeneous, flexible and transparent layer on a surface, as defined above. This is very advantageous for the applications of the present invention. The colloidal sol can comprise for example nanoparticles of a dispersed and metastabilized, porous flexible MOF, for example MIL-89, or any other form mentioned above.

Preferably, the adhesive or binder, when it is used, is selected to be compatible with the MOF, i.e. notably, that it does not alter the MOF itself and/or the catalytic power of the MOF solid.

The adhesive used can be for example an adhesive selected from the group comprising a natural polymer, a synthetic polymer, a clay, a zeolite, a natural resin, a synthetic resin, a glue, an adhesive emulsion, a cement, a concrete or a mixture of two or more of these adhesives.

The binder used can be for example alumina, silica or a mixture of alumina and silica.

The proportion of binder or of adhesive used is such as to permit the desired application on the surface. This proportion can easily be determined by a person skilled in the art.

According to the invention, for application of the MOF solid on a surface, it is possible to use any appropriate technique notably with the MOF selected and the nature and size of the surface to be covered. It can be for example one of the following techniques: simple deposition, for example chemical solution deposition (CSD); spin coating; dip coating; spray coating; wash-coating; roll coating; or any other technique known by a person skilled in the art.

The thickness of the MOF solid on the surface can be any appropriate thickness for application of the present invention. It is not necessary for this thickness to be large, since the catalysis reaction is a surface reaction. In general, a thickness from 0.01 to 100 micrometers, for example from 40 to 1000 nm, for example from 40 to 200 nm is suitable for implementing the present invention.

The MOF solids described in the present text and used in the present invention advantageously have a catalytic manner of functioning, i.e. they return to their initial state after execution of a catalytic cycle, notably of decomposition, removal or reduction of nitrogen oxide, without deterioration or modification of the MOF, at low temperature and without using reducing agents. Their service life in the use of the present invention is therefore remarkable, in contrast to the catalysts of the prior art, and is particularly suitable for the intended applications described in the present text.

When the present invention is applied in a device, the form of the MOF solid is selected so as to enable said device to promote, preferably optimize, contact between the effluent to be treated and the MOF solid. The device itself is preferably constituted for promoting, preferably optimizing, contact between the effluent and the MOF solid. The device preferably makes it possible to bring the MOF solid in contact with the effluent in order to oxidize, or even remove the nitrogen oxide that it contains.

The present invention therefore also relates to a device for removing nitrogen oxide, said device comprising an MOF solid as defined above, and means for bringing said MOF solid into contact with the nitrogen oxide.

For example, the means for contacting the MOF solid with the nitrogen oxide can be means for bringing the MOF solid into contact with a liquid or gaseous effluent comprising said nitrogen oxide. The effluent can be as defined above. The MOF solid can be in a form as defined above. The device then makes it possible, for example, to remove the nitrogen oxide from the effluent by simple contact with the MOF that it contains.

The device of the present invention can be in the form of a catalytic converter of a vehicle, in the form of a device for filtration and/or purification of the effluents from a building, parking lot, tunnel, factories, laboratories, incineration plants, hydrocarbon refineries, etc., and any other example whether or not mentioned in the present text.

When the present invention is applied in a device forming a catalytic converter, the latter can be constituted for example of a monolith of metal or of ceramic or of any other appropriate material, for example structured as a honeycomb, which contains the MOF according to the present invention on its walls, said monolith being protected by a stainless steel casing. The monolith can be composed for example of fine longitudinal channels separated by thin walls. The MOF of the active phase can be deposited on the ceramic or metal support for example by impregnation, for example by a method called wash-coating. The active phase can form a thin layer, for example from 0.5 to 200 μm, on the inside walls of the channels.

The various types of monoliths that are currently available commercially can be used for implementing the present invention. For catalytic converters, the models with 90 or more often 60 channels per cm² (i.e. 600 or more often 400 cpsi) can be used, for example. In this case the channels have a size of about 1 mm and the walls have thicknesses of about 0.15 mm.

According to the invention, for application of the MOFs for removal of the NOx emitted by vehicles, the De-NOx function of the catalytic converter can also be separated from the rest of an already existing purification line. A monolithic module coated with MOF catalysts according to the present invention can for example be added, notably at the end of the line, where the exhaust gases are at a lower temperature. In this example, there is advantageously a dual objective: not to reach the temperature limit of stability of the hybrid structure of the MOF and to be in the zone of maximum performance of application of the invention. Moreover, this position allows the device of the invention to remove the nitrogen oxides produced by the engine and in addition, if applicable, by the oxidizing elements of an oxidation catalyst and/or of a particulate filter. An advantageous position of the device of the invention may be for example the muffler, where the temperature of the exhaust gases is lower.

The present invention makes it possible in general to remove pollutants such as nitrogen oxides, resulting from the combustion of hydrocarbons, coal, fuels from biomass, in the presence of air or from the oxidation of nitrogen compounds, emitted by vehicles, factories, workshops, stored products, etc., efficiently, at lower cost and without using reducing agents.

The present invention, as well as the device for decomposition or removal of nitrogen oxides of the present invention, can be used on stationary sources of nitrogen oxide, for example chemical plants, for example manufacturing nitric acid, fertilizers or other nitrogen-containing products, units for refining and processing petroleum products, processing plants, iron and steel works, factories for agricultural products and foodstuffs, power stations generating electricity by combustion, glassworks, cement works, incinerators, for example of household and/or industrial and/or hospital waste, units for generation or co-generation of heat, including boilers in residential accommodation, private or public buildings, communities, hospitals, schools, retirement homes, etc., workshops and kitchens.

The present invention, as well as the device for decomposition or removal of nitrogen oxides of the present invention, are also applicable to mobile sources, for example vehicles with a heat engine or a hybrid system that has a thermal unit, notably operating on gasoline, diesel, gas, alcohol, coal, biofuels, aircraft, cars, trucks, tractors, agricultural machinery, industrial machines, utility vehicles, non-electric trains, motor boats of any size, dimensions and uses.

The present invention, as well as the device for decomposition or removal of nitrogen oxides of the present invention, are also applicable to systems for ventilation and/or air conditioning, in order to purify the air entering these systems and/or leaving these systems. They apply to the systems for ventilation and/or air conditioning of public or private buildings, residential accommodation, communities, offices, factories, industrial installations, workshops, care centers, hospitals, schools, training centers, research centers, barracks, hotels, commercial centers, stadiums, cinemas, theaters, parking lots, vehicles, boats, trains, aircraft, etc.

Another advantage of the present invention is that the MOF solids, notably those presented above, can be recycled after being used as catalysts according to the present invention, for example if they deteriorate, as a result of their use, or due to wear, notably over time, presence of poisons in the gases to be treated, etc., for example also, quite simply for recycling them.

Thus, for example when they have been used in a filter for removing nitrogen oxides from a gaseous or liquid effluent, or when they have simply been mixed with an effluent, from which the nitrogen oxide has thus been removed, the MOFs can be recovered, then degraded so as to recover their constituents, for example for making new MOFs or other materials. Recovery can be effected by simple decanting or filtration of a liquid effluent containing the MOF, or by recovery of the MOF that is present in a device for removing nitrogen oxide. For degradation of the MOF solid recovered, for example, it is possible to use an aqueous acid solution, preferably strong, for example HCl, for example 1 to 5M, optionally with heating, for example from 30 to 100° C. On an MOF solid with a metal M, for example, this makes it possible to precipitate M, for example Fe, and thus recover the metallic part of the MOF. An example of recycling of an MOF is given below. The components of the porous hybrid solid (MOF) in the above example are recovered after catalysis of nitrogen oxide removal. The use of the present invention therefore is of a certain ecological character that integrates perfectly in the current trend of environmental protection and sustainable development.

Another advantage of the present invention is that the MOF solids, notably those presented above, can be recovered after being used as catalysts according to the present invention.

Thus, for example when the MOF solids are used in a filter for removing nitrogen oxides from a gaseous or liquid effluent, or when they have simply been mixed with an effluent, from which the nitrogen oxide has thus been removed, they may be “poisoned” by chemical species such as sulfur compounds, heavy hydrocarbons, soot, etc. Regeneration, according to the present invention, consists of withdrawing these chemical species from the MOFs. This regeneration can be effected for example by simple heating under vacuum or under a stream of inert gas, for example N₂, Ar, Ne, etc. In cases where the impurities are more stably coordinated, this regeneration can be effected for example by suspension in alcohol, for example in methanol, ethanol, propanol or any other suitable alcohol or a mixture of two or more of said alcohols, optionally by heating, for example at a temperature from 50 to 100° C., for example from 70 to 90° C., for a duration permitting regeneration of the MOF, for example from 15 minutes to 5 hours, for example from 1 to 3 hours, for example for 2 hours. This regeneration can also be effected for example by means of a stream of steam, preferably supported by an inert gas, preferably at a temperature from 80 to 100° C., for example from 15 minutes to 5 hours, for example from 1 to 3 hours, for example for 2 hours. The latter method advantageously makes it possible to treat the MOF when it is put in a device for it to be used according to the present invention, without having to dismantle the device. Once again, use of the present invention displays a certain ecological character which integrates perfectly in the current trend of environmental protection.

Other advantages can be seen by a person skilled in the art on reading the examples given below, illustrated by the appended drawings, which are nonlimiting and are for purposes of illustration.

### BRIEF DESCRIPTION OF THE DRAWINGS

FIG. 1 shows an X-ray diffraction pattern (Lambda Cu=1.5406 angstroms) of the solid MIL-100(Fe).

FIG. 2 shows a nitrogen adsorption isotherm at 77 K of the dehydrated solid MIL-100 (P₀=1 atm).

FIG. 3 shows a thermogravimetric analysis (in air, with a heating rate of 2° C./minute) of the compound MIL-100(Fe).

FIG. 4 shows the structure of the solid MIL-100(Fe). (a): trimer of octahedron and trimesic ligand; (b): supertetrahedron; (c): structure 3d, schematic; (d): the two types of mesoporous cages.

FIG. 5 shows, left: the variation of the proportion of iron(II) in MIL-100(Fe) as a function of the activation temperature under vacuum by Mossbauer spectroscopy; right: X-ray thermodiffraction under vacuum of the solid MIL-100(Fe) (λ_{Cu}=1.5406 Å).

FIG. 6 is a synoptic diagram of activation of the solid MIL-100(Fe).

FIG. 7 shows a graph of an investigation of Brønsted acid strength of the —OH groups of different species grafted on the sample MIL-100(Cr) analyzed by IR after adsorption of CO: correlation between the displacement ν(OH), values of HO and position ν(CO).

FIG. 8 shows the X-ray diffraction pattern of the solid MIL-101(Fe) (λ_{Cu}=1.5406 Å).

FIG. 9 shows a thermogravimetric analysis (in air, with a heating rate of 2° C./minute) of the compound MIL-101(Fe).

FIG. 10 shows X-ray diffraction patterns of the raw solid MIL-88A (upper curve) and suspended in water (lower curve).

FIG. 11 shows a thermogravimetric analysis (in air, with a heating rate of 2° C./minute) of the hydrated compound MIL-88A(Fe).

FIG. 12 shows X-ray diffraction patterns of the dry solid MIL-88B (curve (b), bottom) and hydrated (curve (a), top).

FIG. 13 shows a thermogravimetric analysis (in air, with a heating rate of 2° C./minute) of the hydrated compound MIL-88B.

FIG. 14 shows X-ray diffraction patterns of the dry solid MIL-89 (curve a), DMF (curve b) and hydrated (curve c).

FIG. 15 shows an X-ray diffraction pattern of the solid MIL-88C.

FIG. 16 shows a thermogravimetric analysis (in air, with a heating rate of 2° C./minute) of the compound MIL-88C, raw from synthesis.

FIG. 17 shows X-ray diffraction patterns of the raw solid MIL-88D (curve (b), bottom) and hydrated (curve (a), top).

FIG. 18 shows a thermogravimetric analysis (in air, with a heating rate of 2° C./minute) of the hydrated compound MIL-88D(Fe).

FIG. 19 shows X-ray diffraction patterns of the raw solid MIL-88B-NO₂ (curve (a), top) and hydrated (curve (b), bottom).

FIG. 20 shows a thermogravimetric analysis (in air, with a heating rate of 2° C./minute) of the compound MIL-88B-NO₂(Fe) after washing and drying.

FIG. 21 shows X-ray diffraction patterns of the raw solid MIL-88B-2OH (curve (c), bottom), hydrated (curve (b), middle) and dried under vacuum (curve (a), top).

FIG. 22 shows a thermogravimetric analysis (in air, with a heating rate of 2° C./minute) of the hydrated compound MIL-88B-2OH(Fe).

FIG. 23 shows X-ray diffraction patterns of the raw solid MIL-88B-NH₂ (curve (b), bottom) and of the dry solid MIL-88B-NH₂ (curve (a), top).

FIG. 24 shows a thermogravimetric analysis (in air, with a heating rate of 2° C./minute) of the hydrated compound MIL-88B-NH₂(Fe).

FIG. 25 shows X-ray diffraction patterns of the raw solid MIL-88B-Cl (curve (b), bottom) and hydrated (curve (a), top).

FIG. 26 shows a thermogravimetric analysis (in air, with a heating rate of 2° C./minute) of the hydrated compound MIL-88B-Cl(Fe).

FIG. 27 shows X-ray diffraction patterns of the raw solid MIL-88B-4-CH₃ (curve (b), bottom) and hydrated (curve (a), top).

FIG. 28 shows a thermogravimetric analysis (in air, with a heating rate of 2° C./minute) of the hydrated compound MIL-88B-4-CH₃(Fe).

FIG. 29 shows X-ray diffraction patterns of the raw solid MIL-88B-4F (curve (c), bottom), hydrated (curve (b)) and solvated with EtOH (curve (a), top).

FIG. 30 shows a thermogravimetric analysis (in air, with a heating rate of 2° C./minute) of the hydrated compound MIL-88B-4F (Fe).

FIG. 31 shows X-ray diffraction patterns of the raw solid MIL-88B-Br (curve (b), bottom) and hydrated (curve (a), top).

FIG. 32 shows a thermogravimetric analysis (in air, with a heating rate of 2° C./minute) of the hydrated compound MIL-88B-Br(Fe).

FIG. 33 shows X-ray diffraction patterns of the raw solid MIL-88F (curve (b), bottom) and hydrated (curve (a), top).

FIG. 34 shows a thermogravimetric analysis (in air, with a heating rate of 2° C./minute) of the hydrated compound MIL-88F (Fe).

FIG. 35 shows an X-ray diffraction pattern of the raw solid MIL-88G (curve (c), bottom), solvated with DMF (curve (b), middle) and solvated with pyridine (curve (a), top).

FIG. 36 shows a thermogravimetric analysis (in air, with a heating rate of 2° C./minute) of the compound MIL-88G (Fe), raw from synthesis.

FIG. 37 shows an X-ray diffraction pattern of the raw solid MIL-88G-2Cl (curve (b), bottom) and dry (curve (a), top).

FIG. 38 shows a thermogravimetric analysis (in air, with a heating rate of 2° C./minute) of the compound MIL-88G-2Cl (Fe), raw from synthesis.

FIG. 39 shows X-ray diffraction patterns of the raw solid MIL-102(Fe) (curve (a)) and reference MIL-102 (Cr) (curve (b)).

FIG. 40 shows a thermogravimetric analysis (in air) of the compound MIL-102 (Fe), raw from synthesis.

FIG. 41 is a schematic diagram of the crystallographic structure of MIL-126(Fe). The FeO₆ polyhedra are shown with or without a star, indicating the two MIL-88D frameworks. The carbon atoms are shown in black.

FIG. 42 shows an X-ray diffraction pattern of MIL-126(Fe) (λCu=1.5406 Å).

FIG. 43 shows the results in graph form of a thermogravimetric analysis of MIL-126(Fe) in air (heating rate 2° C./min).

FIG. 44 shows a nitrogen adsorption isotherm of MIL-126(Fe) (P₀=1 atmosphere).

FIG. 45 shows an X-ray diffraction pattern of iron 3,3′,5,5′-azobenzenetetracarboxylate (MIL-127(Fe)) (λCu=1.5406 Å).

FIG. 46 shows the thermogravimetric analysis of iron 3,3′,5,5′-azobenzenetetracarboxylate in air (heating rate 2° C./min).

FIG. 47 shows a nitrogen adsorption isotherm of iron 3,3′,5,5′-azobenzenetetracarboxylate (P₀=1 atmosphere).

FIG. 48 shows a reaction scheme for obtaining 3,5,3′,5′-tetramethylbiphenyl-4,4′-dicarboxylic acid.

FIG. 49 shows a reaction scheme for obtaining 3,3′-dimethylbiphenyl-4,4′-dicarboxylic acid.

FIG. 50 shows an SEM (Scanning Electron Microscopy) micrograph of the solid MIL-89 nano.

FIG. 51 shows an SEM (Scanning Electron Microscopy) micrograph of the solid MIL-88Anano.

FIG. 52 shows an SEM (Scanning Electron Microscopy) micrograph of the solid MIL-100 nano.

FIG. 53 shows an SEM micrograph of the solid MIL-88Btnano.

FIG. 54 shows an SEM micrograph of the solid MIL-88Bnano.

FIG. 55 shows a quantity of unsaturated iron sites present in MIL-100 Fe activated under vacuum at different temperatures.

FIG. 56 shows a schematic view of the phenomenon of respiration (swelling and contraction) in the solids MIL-88A, MIL-88B, MIL-88C, MIL-88D and MIL-89. The amplitude of swelling between dry forms (top) and open forms (bottom) is shown as a percentage at the bottom of the figure.

FIG. 57 shows an explanatory diagram of flexibility in the hybrid phases MIL-53 (a) and MIL-88 (b and c).

FIG. 58 shows, top, an investigation of the reversibility of the swelling of the solid MIL-88A by X-ray diffraction (λ˜1.79 Å), bottom, X-ray diffraction patterns of the solid MIL-88A in the presence of solvents (λ˜1.5406 Å).

FIG. 59 is a graph showing the experimental results of conversion of NOx to N₂ and N₂O in the presence of water on sample MIL-100 (Fe) as a function of temperature, after activation under dry Ar at 250° C. for 3 hours, in the steady state, after saturation of the storage step.

FIG. 60 is a graph showing the experimental results of conversion of NOx to N₂ and N₂O in the presence of oxygen on sample MIL-100 (Fe) as a function of temperature, after activation under dry Ar at 250° C. for 3 hours, in the steady state, after saturation of the storage step.

FIG. 61 shows differential IR spectra of the species adsorbed on the surface of sample MIL-100 (Fe) under a reaction stream of 500 ppm NO+10% O₂ in argon, as a function of temperature and at equilibrium (steady state), after activation under dry Ar at 250° C. for 3 hours.

FIG. 62 is a graph showing the experimental results of conversion of NOx to N₂ and N₂O in the presence of water and oxygen on sample MIL-100 (Fe) as a function of temperature, after activation under dry Ar at 250° C. for 3 hours, in the steady state, after saturation of the storage step.

FIG. 63 is a graph showing the experimental results of conversion of 900 ppm of NO (with or without oxygen and water) at 30° C., after pretreatment of sample MIL-100 (Fe) at 250° C. for 6 hours, at space velocities between 5000 and 20000 h⁻¹.

FIG. 64A is a graph showing the experimental results of conversion of 900 ppm of NO, in the presence or absence of oxygen, at 30° C., on a sample MIL-100 (Fe) after pretreatment of the sample at 250° C. for 6 hours, at space velocities between 5000 and 20000

FIG. 64B shows the percentage conversion of 900 ppm of NO on different samples with iron, at a space velocity of 20000 h⁻¹ in catalysis according to the present invention.

FIG. 64C shows the percentage conversion of 900 ppm of NO on different samples with iron, at a space velocity of 20000 h⁻¹ in catalysis according to the present invention.

FIG. 65 is a graphical representation of the concentration profile during removal of nitrogen oxides (concentration, ppm, of nitrogen oxide as a function of reaction time in min.).

FIG. 66 is a schematic diagram of the formation of thin layers of porous, flexible inorganic-organic hybrid solids.

FIG. 67: (left) X-ray diffraction patterns (λ_{Cu}=1.5406 Å) of nanoparticles of the solid MIL-89: (A) particles obtained from precursor FeCl₃; (B) particles obtained from iron acetate; (C) photo of a gel of MIL-89 obtained after 10 minutes at 60° C. and 2 days at room temperature; (D) monolith of MIL-89 formed after 10 minutes at 60° C. and 3 months at room temperature.

FIG. 68 is a micrograph from atomic-force electron microscopy of a thin layer of the solid MIL-89.

FIG. 69 shows an X-ray diffraction pattern of the raw solid MIL-88B 4-CH₃ obtained (lower curve) and hydrated (upper curve).

FIG. 70 shows a thermogravimetric analysis in air of the hydrated compound MIL-88B 4-CH₃(Fe) obtained with a heating rate of 2° C./minute).

FIG. 71 shows an X-ray diffraction pattern of the raw solid MIL-88D 4-CH₃ obtained (lower curve) and hydrated (upper curve).

FIG. 72 shows a thermogravimetric analysis in air of the hydrated compound MIL-88B 4-CH₃(Fe) obtained with a heating rate of 2° C./minute.

FIG. 73 shows an X-ray diffraction pattern of the raw solid MIL-88D 2CH₃ obtained (lower curve), hydrated (middle curve) and wetted (upper curve).

FIG. 74 shows a thermogravimetric analysis in air of the hydrated compound MIL-88D 2CH₃ (Fe) obtained with a heating rate of 2° C./minute.

FIG. 75 shows an X-ray diffraction pattern of the nonfluorinated solid MIL-100(Fe) or N-MIL-100(Fe).

FIG. 76 shows a nitrogen adsorption isotherm of the nonfluorinated solid MIL-100(Fe) or N-MIL-100(Fe) (P₀=1 atmosphere).

FIG. 77 shows a diagram of a first example of a suitable device for application of the present invention.

FIG. 78 shows a diagram of a second example of a suitable device for application of the present invention. This figure also shows a photograph of a cross-section of a portion of the device of this example.

FIG. 79 shows schematically a device according to the present invention, installed in the exhaust system of an engine (Mot).

FIG. 80 shows schematically a device according to the present invention, inserted in an exhaust system in the form of a catalytic converter.

FIG. 81 shows schematically a combustion or heat engine in which a device according to the invention is incorporated for removing nitrogen oxides from the engine exhaust gases.

### EXAMPLES

### Example 1

### Synthesis and Data for the Examples of Iron Carboxylates Usable as Catalysts According to the Present Invention

This example describes the synthesis of various metal carboxylates usable for application of the present invention. The solids obtained were characterized according to the methods described below.

The crystalline structure of the iron carboxylate solids was analyzed by X-ray diffraction using a Siemens D5000 diffractometer (CuKα radiation, λ_{Cu}=0.5406 Å, mode θ-2θ), at room temperature in air. The diagrams are shown either as angular distances (2θ, in degrees °) or as interplanar distance (d, in Å (angstroms)).

Characterization of porosity (Langmuir specific surface and pore volume) of the solids was measured by nitrogen adsorption at 77 K with a Micromeretics ASAP-2010 instrument. The solids were dehydrated beforehand at 150° C. under primary vacuum overnight. The isotherm of nitrogen adsorption by the solids is given by a curve representing the volume of nitrogen adsorbed V (in cm³/g) as a function of the ratio of the pressure P to the reference pressure P₀=1 atm.

The thermogravimetric analysis was carried out under air atmosphere using a Model TA-instrument 2050. The heating rate was 2° C./minute. The curve resulting from the thermogravimetric analysis of the solids represents weight loss Pm (%) as a function of the temperature T (in ° C.).

Elemental analysis of the solids was carried out by the Central Analysis Service of the CNRS of Vernaison:

### Organic Analysis:

Microanalyses C, H, N, O, S in the pharmaceutical products, the polymers and generally the synthesis products, by coulometric, katharometric or infrared cell detection.

### Inorganic Analysis:

The main analytical techniques employed in these examples are as follows:
ICP-AES (“Inductive Coupled Plasma—Atomic Emission Spectroscopy”) with different types of detectors;
ICP-MS (“Inductively Coupled Plasma-Mass Spectrometry”) with quadrupole or magnetic-sector mass spectrometers;
CVAAS (“Cold-Vapor Atomic Absorption Spectroscopy”);
Coupled ICP/MS/HPLC (“Inductively Coupled Plasma/Mass Spectrometry/High Performance Liquid Chromatography);
X-ray fluorescence;
Sample treatments by wet process, by dry process or microwaves.
   a) MIL-100(Fe) or Fe₃O[C₆H₃—(CO₂)₃]₂.X.nH₂O (X=F, Cl, OH)

The iron carboxylate MIL-100(Fe) was synthesized according to two conditions: with and without hydrofluoric acid.

Conditions of Synthesis with Hydrofluoric Acid:

56 mg of metallic iron powder (1 mmol, marketed by the company Riedel-de Haën, 99%), 140 mg of 1,3,5-benzenetricarboxylic acid or trimesic acid (0.6 mmol, 1,3,5-BTC; marketed by the company Aldrich, 99%) are dispersed in 5 mL of distilled water with 0.6 mL of 2M nitric acid (marketed by the company VWR, 50%) and 0.4 mL of 5M hydrofluoric acid (marketed by the company SDS, 50%). The whole is put in a 23-ml Teflon container that is put in a metal bomb from the company PAAR and left for 6 days at 150° C. with a plateau of temperature increase of 12 hours and a plateau of temperature decrease of 24 hours. The solid is recovered by filtration.

Then the solid (200 mg) is suspended in 100 mL of water and distilled under reflux with stirring for 3 h to remove the trimesic acid remaining in the pores. The solid is then recovered by hot filtration.

It is then dried overnight at 100° C. in a stove.

Conditions of Synthesis without Hydrofluoric Acid:

0.27 g of FeCl₃.6H₂O (1 mmol, marketed by the company Alfa Aesar, 98%), 140 mg (0.6 mmol) of 1,3,5-benzenetricarboxylic acid (1,3,5-BTC; marketed by the company Aldrich, 99%) are dispersed in 5 mL of distilled water. The whole is left in a 23-ml Teflon container that is put in a PAAR metal bomb for 3 days at 130° C. The solid is then filtered and washed with acetone.

The solid (200 mg) is then suspended in 100 mL of water and distilled under reflux with stirring for 3 h to remove the trimesic acid remaining in the pores. The solid is then recovered by hot filtration.

It is then dried overnight at 100° C. in a stove.

### Characteristic Data of the Iron Carboxylate Solid MIL-100(Fe)

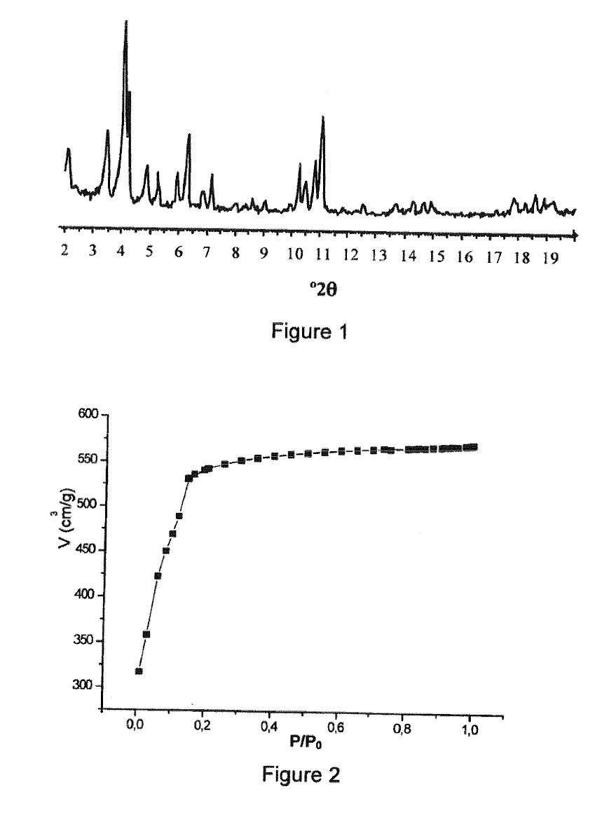

Analysis of the crystalline structure of the solid MIL-100(Fe) by X-ray diffraction gives the X-ray diffraction pattern shown in FIG. 1.

The characteristics of the crystal structure are as follows:
the space group is Fd-3m (No. 227).
the lattice parameters are: a=73.1 Å; unit cell volume V=393000 A³.

The nitrogen absorption isotherm at 77 K of the solid MIL-100(Fe) (at pressure P₀=1 atm) is given in FIG. 2. The (Langmuir) specific surface of this solid is close to 2900 m².g⁻¹.

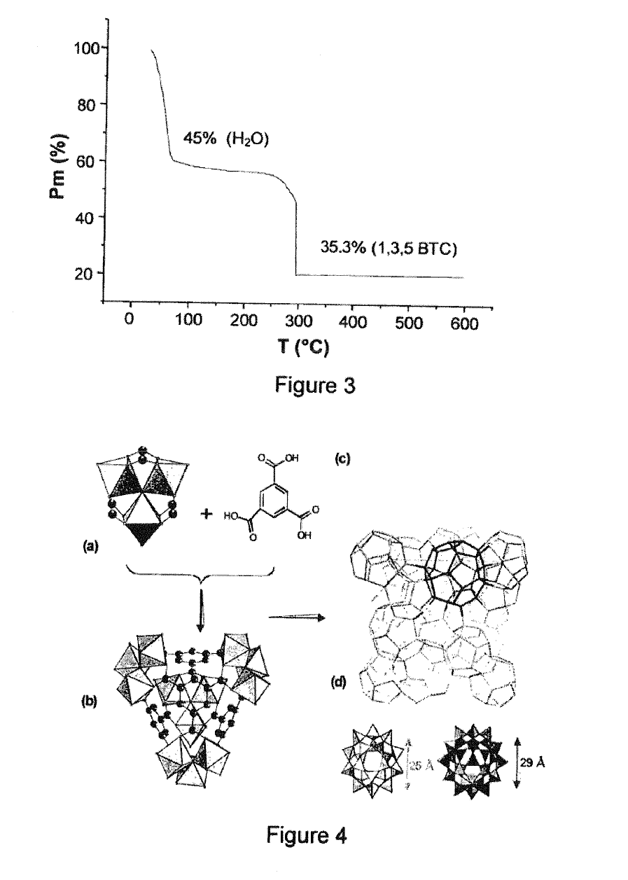

The curve resulting from thermogravimetric analysis of the compound MIL-100(Fe) is given in FIG. 3. This diagram shows the weight loss Pm (%) as a function of the temperature T (in ° C.).

TABLE 1
elemental analysis of the solid MIL-100(Fe) or
Fe₃O[C₆H₃—(CO₂)₃]₂•X•nH₂O
in the case when X = F

| Element | | | |
|---|---|---|---|
| (wt. %) | % Iron | % Carbon | % Fluorine |
| MIL-100(Fe) | 13.8 | 23.5 | 1.3 |

The solid MIL-100, with Fe or Cr, was manufactured as in the documents [70, 83]. It consists of trimers of octahedra of iron or of chromium, following synthesis with Fe or Cr, connected by trimesic acids which combine to form hybrid supertetrahedra. The whole thus leads to a mesoporous crystalline structure, whose cages, of free dimensions 25 and 29 Å, are accessible through microporous windows (FIG. 4). The resultant pore volume is very large, close to 1.2 g.cm⁻³ for a BET specific surface of 2200 m².g⁻¹.

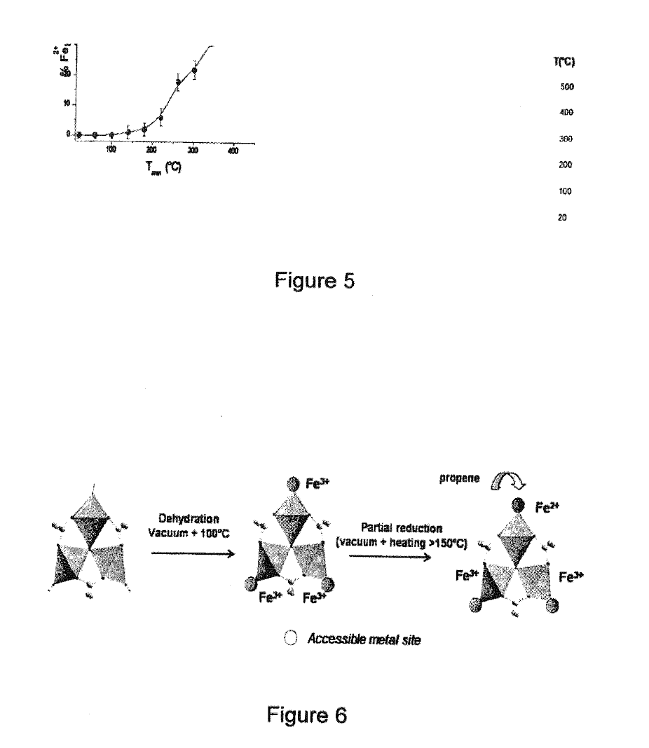

The particular feature of this solid is the stability of its structure after departure of the water coordinated on the metal sites. This phenomenon is described in A. Vimont, et al. J. Am. Chem. Soc, 2006, 128, 3218-3227: First characterization of acid sites in a new chromium(III) dicarboxylate with giant pores [84]. The water is easily evacuated by heating under vacuum and leaves room for unsaturated, accessible metal sites (metal in coordination number five). Moreover, when the activation temperature under vacuum exceeds 150° C., there is partial reduction of iron(III) to iron(II). This reduction takes place increasingly with the temperature and does not destabilize the structure before 280° C., as shown by X-ray thermodiffractometry under vacuum (FIG. 5).

The solid MIL-100(Fe) has the composition Fe^{III}₃O(H₂O)₂F.{C₆H₃—(CO₂)₃}₂.nH₂O (n˜14.5). Its framework is cationic with one compensating anion per iron trimer. Here, the anion is a fluoride which is coordinated on the iron. The stability of MIL-100(Fe) on partial reduction of iron(III) to iron(II) might be explained by departure of conjugated fluorine on reduction of the iron. Thus, it is entirely reasonable to think that one iron(III) per trimer can be reduced to iron(II) at the same time as departure of the fluoride ions to respect electroneutrality. On return to room temperature, in air, the solid reoxidizes with probable coordination of OH anions on the iron. This property has also been observed by the inventors on vanadium.

This property is fundamental and to the best knowledge of the inventors is unique in the field of MOFs: reduction of an unsaturated metal site under vacuum while maintaining integrity of the structure. What are its consequences? The iron(II) and iron(III) unsaturated metal sites possess an electron acceptor character (Lewis acid) and form n complexes with molecules possessing an electron donor character (Lewis base), such as alkenes or alkynes or nitrogen oxides. The stabilization of the complexes thus formed can be explained on the basis of the Dewar-Chatt-Duncanson model described in J. Chatt and L. A. Duncanson, J. Chem. Soc, 1953, 2939-2947 (“Olefin co-ordination compounds. Part III. Infrared spectra and structure: attempted preparation of acetylene complexes”) [85] considering on the one hand the electronic structure of the double or triple carbon-carbon bond of the olefin and on the other hand the vacant orbitals of the adsorption site: the bond between alkene or alkyne implies (i) a delocalization of the electrons of the binding π orbitals from the unsaturated hydrocarbon to the vacant orbitals of the adsorption site (donor-acceptor interaction by σ bond) (ii) a delocalization of the electrons of the partially filled d orbitals from the adsorption site to the antibinding π* orbitals of the unsaturated hydrocarbon (π bond). Iron(II) possesses an additional d electron relative to iron(III), which reinforces the π bond with the hydrocarbon and thus increases the stability of the complex formed. Thus, the partially reduced MIL-100(Fe) will be able to interact more strongly with such molecules (FIG. 6). Similar phenomena can be envisaged between the π orbitals of NO or NO₂ and the d orbitals of iron, as well as interactions of donation between the free doublet of electrons on the nitrogen of these molecules and the empty d orbitals of the iron.

Finally, although MIL-100 possesses unsaturated metal sites (iron) which are Lewis acid sites, even if the latter can transform to Brønsted acidity by coordination of proton donor molecules such as water (A. Vimont et al., Journal of Physical Chemistry C, 111 (2007), 383-388: Creation of Controlled Brønsted Acidity on a Zeotypic Mesoporous Chromium(III) Carboxylate by Grafting Water and Alcohol Molecules [86]), the acidity measured by CO adsorption is not very strong and this undoubtedly does not cause conversion of NOx to nitric acid in the pores.

In fact, our previous studies on an MIL-100(Cr) system showed the presence of a large number of Lewis acid sites [84]), which are transformed to Brønsted acid sites by adsorption of water. The addition of small calibrated doses of CO confirmed the existence of two types of water molecules with similar acid strength, overall quite low.

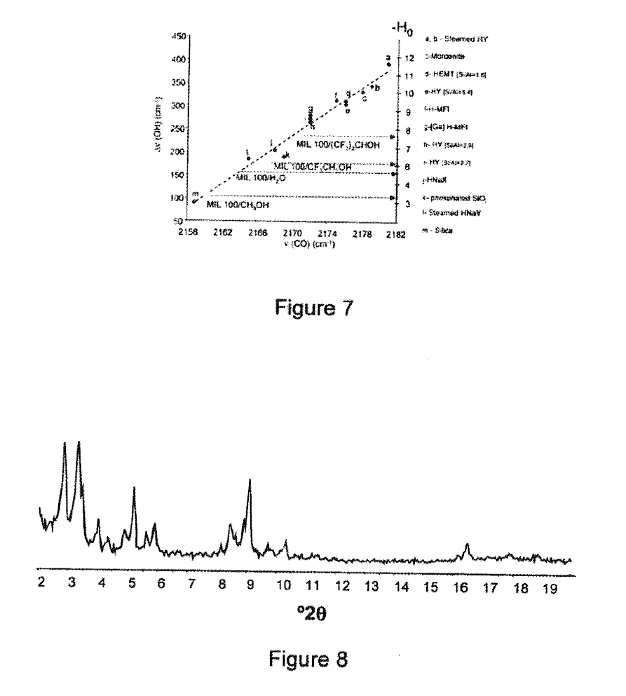

The introduction of alcohols of different basicity showed that the strength of the Brønsted acid sites depends on the nature of the coordinated molecule and increases with the basicity of the protic species. The experimental results of this introduction are shown in the accompanying FIG. 7.

b) MIL-101(Fe) or Fe₃O[C₆H₄—(CO₂)₂]₃.X.nH₂O (X=F, Cl, OH)

### Synthesis of the Solid MIL-101(Fe):

0.27 g (1 mmol) of FeCl₃.6H₂O, 249 mg (1.5 mmol) of 1,4-benzenedicarboxylic acid (1,4-BDC, marketed by the company Aldrich, 99%) are dispersed in 10 mL of dimethylformamide (DMF, marketed by the company Fluka, 98%). The mixture is left for 12 hours at 100° C. in a 23-ml Teflon container that is put in a PAAR metal bomb. The solid is then filtered and washed with acetone.

### Characteristic Data of the Solid MIL-101(Fe):

The X-ray diffraction pattern of the solid MIL-101(Fe) is shown in FIG. 8.

The characteristics of the crystal structure are as follows:
the space group is Fd-3m (No. 227).
the lattice parameters of the solid MIL-101(Fe) at 298 K are: a=89.0 Å; unit cell volume V=707000 Å.

The theoretical elemental composition of the dry solid (with X=F) is as follows: Fe 24.2%; C 41.4%; F 2.70; H 1.70.

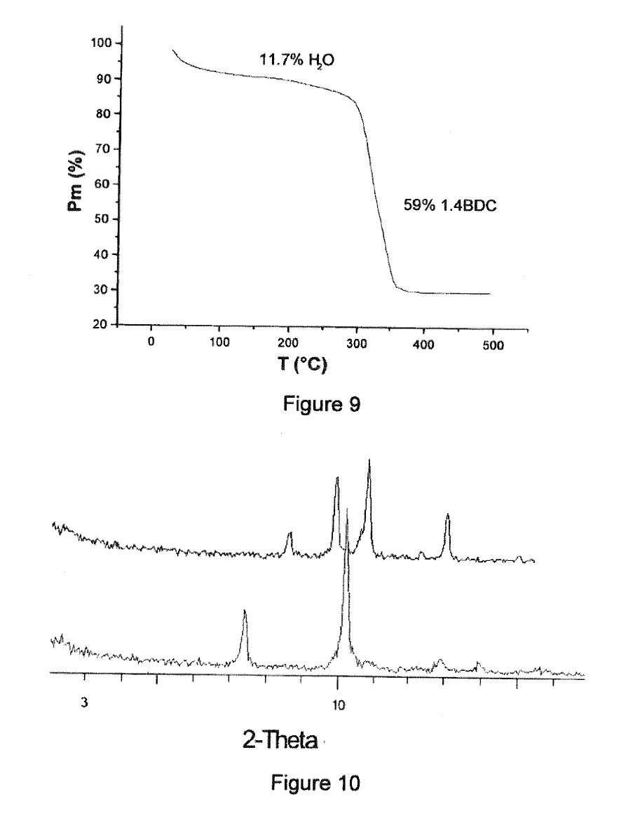

The results of thermogravimetric analysis of the compound MIL-101(Fe), carried out in air, at a heating rate of 2° C./minute, are shown in FIG. 9. The weight loss Pm (%) is shown as a function of the temperature T (in ° C.).

c) MIL-88A(Fe) or Fe₃O[C₂H₂—(CO₂)₂]₃.X.nH₂O (X=F, Cl, OH)

### Synthesis of the Solid MIL-88A(Fe):

0.27 g (1 mmol) of FeCl₃.6H₂O (marketed by the company Alfa Aesar, 98%) and 116 mg (1 mmol) of fumaric acid (Aldrich, 99%) are dispersed in 5 ml of dimethylformamide (DMF, Fluka, 98%) with 0.4 mL of 2M NaOH (Alfa Aesar, 98%). The mixture is left in a 23-ml Teflon container that is put in a PAAR metal bomb for hours at 100° C. The solid is then filtered and washed with acetone.

Then the solid (200 mg) is suspended in 100 mL of distilled water with stirring for 12 h to remove the solvent remaining in the pores. The solid is then recovered by filtration.

### Characteristic Data of the Solid MIL-88A(Fe):

Analysis of the crystal structure of the solid gives the characteristics presented in the following table:

TABLE 2
lattice parameters of the solid MIL-88A, dry and hydrated.

| | | | Unit cell | | |
|---|---|---|---|---|---|
| | a | c | volume | Pore size | Space |
| Phase | (Å) | (Å) | (Å³) | (Å) | group |
| MIL-88A dry | 9.25 | 15.30 | 1135 | | P-62c |
| MIL-88A | 13.9 | 12.66 | 2110 | 6-7 | P-62c |
| hydrated | | | | | |
| (H₂O) | | | | | |

The X-ray diffraction pattern is shown in FIG. 10.

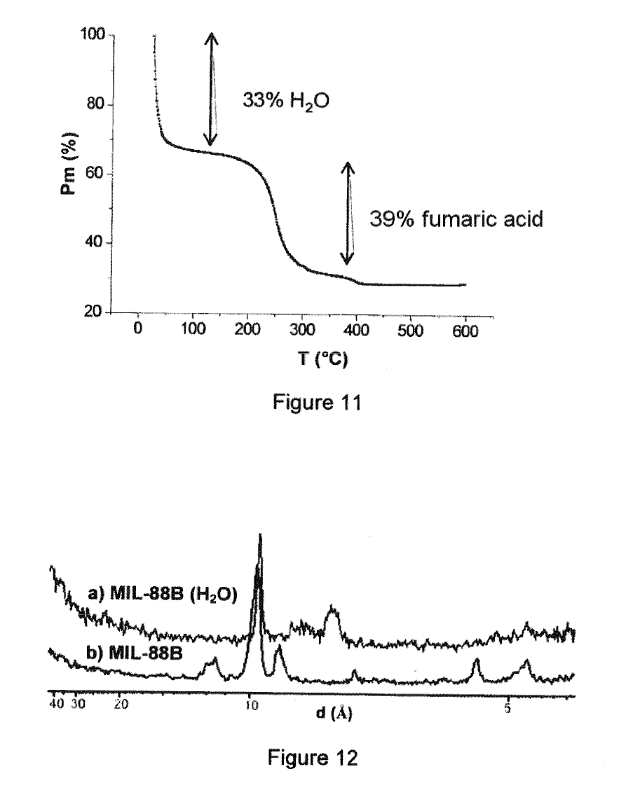

The results of thermogravimetric analysis of the hydrated compound MIL-88A (in air, at a heating rate of 2° C./minute) are shown in FIG. 11. The weight loss Pm (%) is shown as a function of the temperature T (in ° C.).

The compound MIL-88A does not have a surface accessible (greater than 20 m²/g) to nitrogen at 77 K, since the dry structure has a pore size that is too small to incorporate nitrogen N₂.

The elemental analysis is shown in the following table:

TABLE 3

| Element | | |
|---|---|---|
| (wt. %) | % Iron | % Carbon |
| MIL-88A (raw) | 21.8 | 24.0 |

d) MIL-88B(Fe) or Fe₃O[C₆H₄—(CO₂)₂]₃.X.nH₂O (X=F, Cl, OH)

Synthesis of the Solid MIL-88B(Fe):

0.27 g (1 mmol) of FeCl₃.6H₂O (Alfa Aesar, 98%) and 116 mg (1 mmol) of 1,4-benzenedicarboxylic acid (Aldrich, 98%) are dispersed in 5 mL of dimethylformamide (DMF, Fluka, 98%) with 0.4 mL of 2M soda (Alfa Aesar, 98%). The mixture is left in a 23-ml Teflon container that is put in a PAAR metal bomb for 12 hours at 100° C. The solid is then filtered and washed with acetone.

200 mg of the solid is suspended in 100 mL of distilled water with stirring for 12 h to remove the solvent remaining in the pores. Then the solid is recovered by filtration.

### Characteristic Data of the Solid MIL-88B(Fe):

Analysis of the crystal structure of the solid gives the characteristics presented in the following table:

TABLE 4
lattice parameters of the solid MIL-88B, dry and hydrated.

| | | | Unit cell | | |
|---|---|---|---|---|---|
| | a | c | volume | Pore size | Space |
| Phase | (Å) | (Å) | (Å³) | (Å) | group |
| MIL-88B dry | 9.6 | 19.1 | 1500 | <3 | P-62c |
| MIL-88B | 15.7 | 14.0 | 3100 | 9 | P-62c |
| hydrated | | | | | |
| (EtOH) | | | | | |

FIG. 12 shows the X-ray diffraction patterns of the dry solid (bottom, (b)) and of the hydrated solid, (top, (a)).

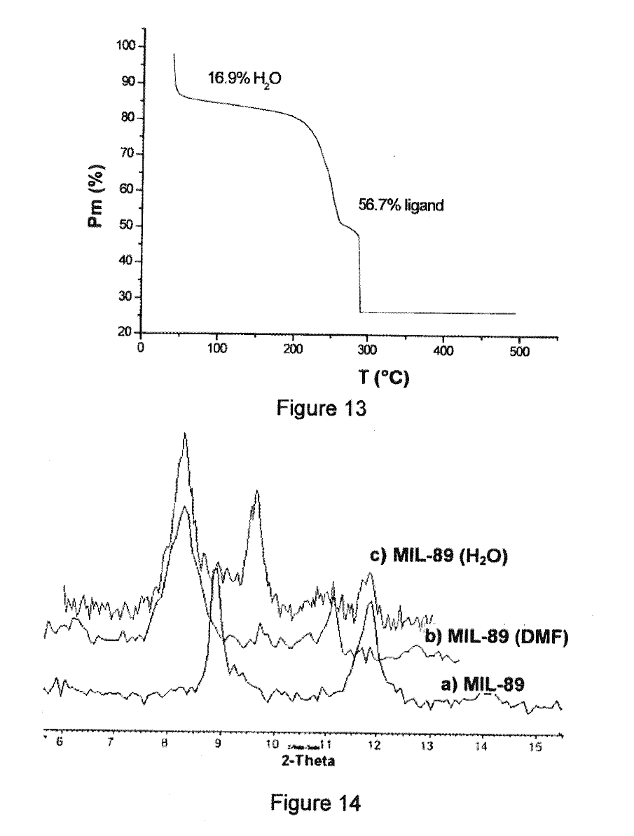

The results of thermogravimetric analysis of the hydrated compound MIL-88B (in air, at a heating rate of 2° C./minute) are shown in FIG. 13. The weight loss Pm (%) is shown as a function of the temperature T (in ° C.).

The compound MIL-88B does not have a surface accessible (greater than 20 m²/g) to nitrogen at 77 K, since the dry structure has a pore size that is too small to incorporate nitrogen N₂.

e) MIL-89 (Fe) or Fe₃O[C₄H₄—(CO₂)₂]₃.X.nH₂O (X=F, Cl, OH)

### Synthesis of the Solid MIL-89(Fe):

172 mg (1 mmol) of iron acetate (prepared following the synthesis described by Dziobkowski et al., Inorg. Chem. 1982, 20, 671 [87]) and 150 mg (1 mmol) of muconic acid (Fluka, 97%) are dispersed in 10 ml of methanol (Fluka, 98%) with 0.35 mL of 2M soda (Alfa Aesar, 98%). The mixture is left in a 23-ml Teflon container that is put in a PAAR metal bomb for 3 days at 100° C. The solid is then filtered and washed with acetone.

200 mg of the solid is suspended in 100 mL of distilled water with stirring for 12 h to remove the solvent remaining in the pores. The solid is then recovered by filtration.

This solid is highly flexible and can swell reversibly up to 160 vol. %, with a gate size of about 11 angstrom.

### Characteristic Data of the Solid MIL-89(Fe):

FIG. 14 shows the X-ray diffraction patterns a), b) and c) respectively of the dry solid MIL-89(Fe), of the solid MIL-89(Fe) solvated with DMF and of the hydrated solid MIL-89(Fe).

The compound MIL-89(Fe) does not have a surface accessible (greater than 20 m²/g) to nitrogen at 77 K, since the dry structure has a pore size that is too small to incorporate nitrogen N₂.

f) MIL-88C(Fe) or Fe₃O[C₁₀H₆—(CO₂)₂]₃.X.nH₂O (X=F, Cl, OH)

### Synthesis of the Solid MIL-88C(Fe):

172 mg (1 mmol) of iron acetate (synthesized according to example 2) and 140 mg (1 mmol) of 2,6-naphthalenedicarboxylic acid (Aldrich, 95%) are dispersed in 5 ml of dimethylformamide (DMF, Fluka, 98%). The mixture is left in a 23-ml Teflon container that is put in a PAAR metal bomb for 3 days at 150° C. with a plateau of temperature increase of 12 hours and a plateau of temperature decrease of 24 hours. The solid is recovered by filtration. The solid is dried at 150° C. under air for 15 hours.

### Characteristic Data of the Solid MIL-88C(Fe):

Analysis of the crystal structure of the solid gives the characteristics presented in the following table:

TABLE 5
lattice parameters of the solid MIL-88C, dry and solvated.

| | | | Unit cell | | |
|---|---|---|---|---|---|
| | a | c | volume | Pore size | Space |
| Phase | (Å) | (Å) | (Å³) | (Å) | group |
| MIL-88C dry | 9.9 | 23.8 | 2020 | 3 | P-62c |
| MIL-88C | 18.7 | 18.8 | 5600 | 13 | P-62c |
| solvated | | | | | |
| (Pyridine) | | | | | |

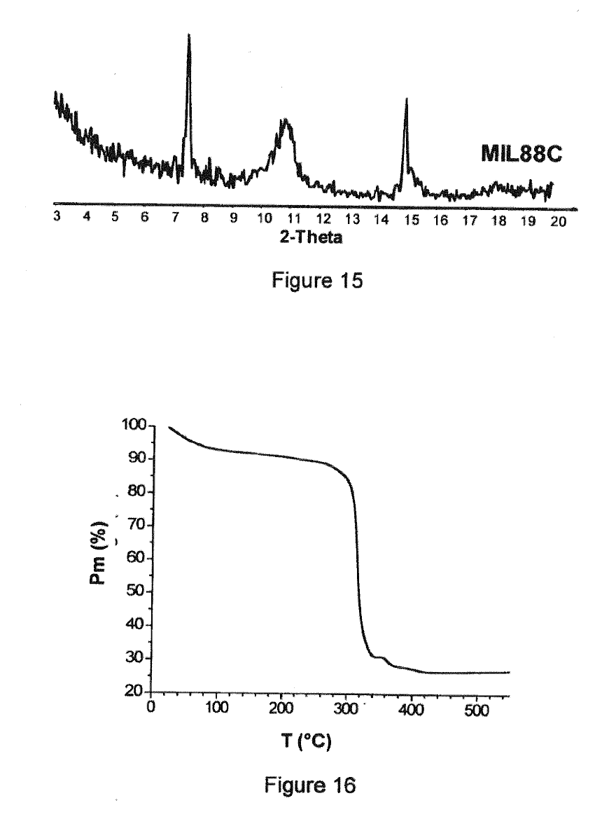

FIG. 15 shows the X-ray diffraction pattern of the solid MIL-88C.

The results of thermogravimetric analysis of the compound MIL-88C, raw from synthesis (in air, at a heating rate of 2° C./minute) are shown in FIG. 16.

This compound does not have a surface accessible (greater than 20 m²/g) to nitrogen at 77 K, since the dry structure has a pore size that is too small to incorporate nitrogen N₂.

g) MIL-88D(Fe) or Fe₃O[C₁₂H₈—(CO₂)₂]₃.X.nH₂O (X=F, Cl, OH)

### Synthesis of the Solid MIL-88D(Fe):

270 mg (1 mmol) of FeCl₃.6H₂O (Alfa Aesar, 98%) and 140 mg (0.6 mmol) of 4,4′-biphenyldicarboxylic acid (Fluka, 95%) are dispersed in 5 ml of dimethylformamide (DMF, Aldrich, 99%). The mixture is left in a 23-ml Teflon container that is put in a PAAR metal bomb for hours at 100° C. with a plateau of temperature increase of one hour and a plateau of temperature decrease of one hour. The solid is recovered by filtration.

The solid is then dried at 150° C. under air for 15 hours.

### Characteristic Data of the Solid MIL-88D(Fe):

Analysis of the crystal structure of the solid gives the characteristics presented in the following table:

TABLE 6
lattice parameters of the solid MIL-
88D, dry and solvated (pyridine).

| | | | Unit cell | | |
|---|---|---|---|---|---|
| | a | c | volume | Pore size | Space |
| Phase | (Å) | (Å) | (Å³) | (Å) | group |
| MIL-88D dry | 10.1 | 27.8 | 2480 | <3 | P-62c |
| MIL-88D | 20.5 | 22.4 | 8100 | 16 | P-62c |
| solvated | | | | | |
| (pyridine) | | | | | |

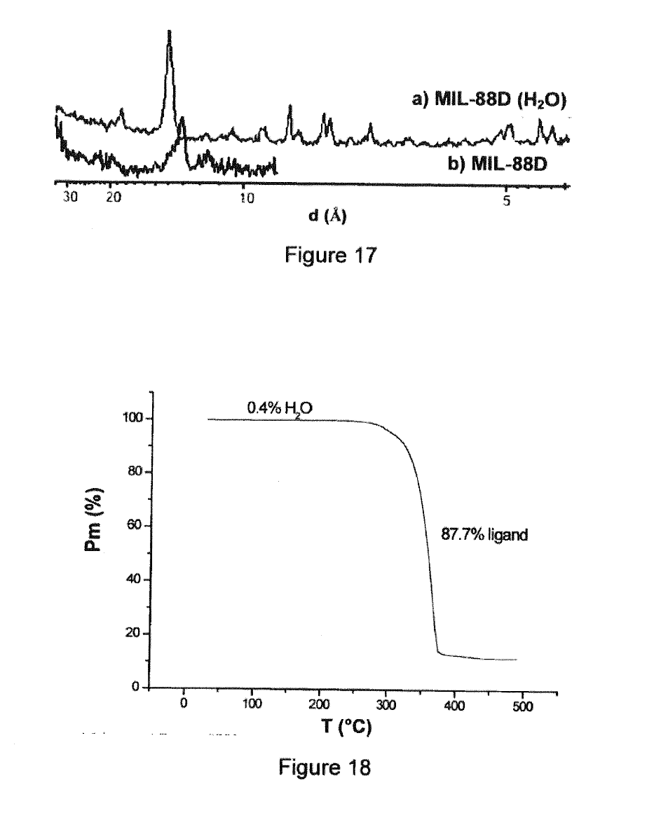

FIG. 17 shows the X-ray diffraction pattern of the solid MIL-88D, raw (curve (b), bottom) and hydrated (curve (a), top).

The results of thermogravimetric analysis of the compound MIL-88D(Fe), hydrated (in air, at a heating rate of 2° C./minute) are shown in FIG. 18 (weight loss Pm as a function of the temperature T in ° C.).

This compound does not have a surface accessible (greater than 20 m²/g) to nitrogen at 77 K, since the dry structure has a pore size that is too small to incorporate nitrogen N₂.

h) MIL-88B-NO₂ (Fe) or Fe₃O[C₆H₃NO₂—(CO₂)₂]₃.X.nH₂O (X=F, Cl, OH)

### Synthesis of the Solid MIL-8813-NO₂(Fe):

0.27 g (1 mmol) of FeCl₃.6H₂O (Alfa Aesar, 98%) and 211 mg (1 mmol) of 2-nitroterephthalic acid (Acros, 99%) are dispersed in 5 ml of distilled water. The mixture is left in a 23-ml Teflon container that is put in a PAAR metal bomb for 12 hours at 100° C. The solid is recovered by filtration.

200 mg of the solid is suspended in 10 mL of absolute ethanol in a 23-ml Teflon container that is put in a PAAR metal bomb for 12 hours at 100° C. to remove the acid remaining in the pores. The solid is then recovered by filtration and dried at 100° C.

### Characteristic Data of the Solid MIL-88B-NO₂(Fe):

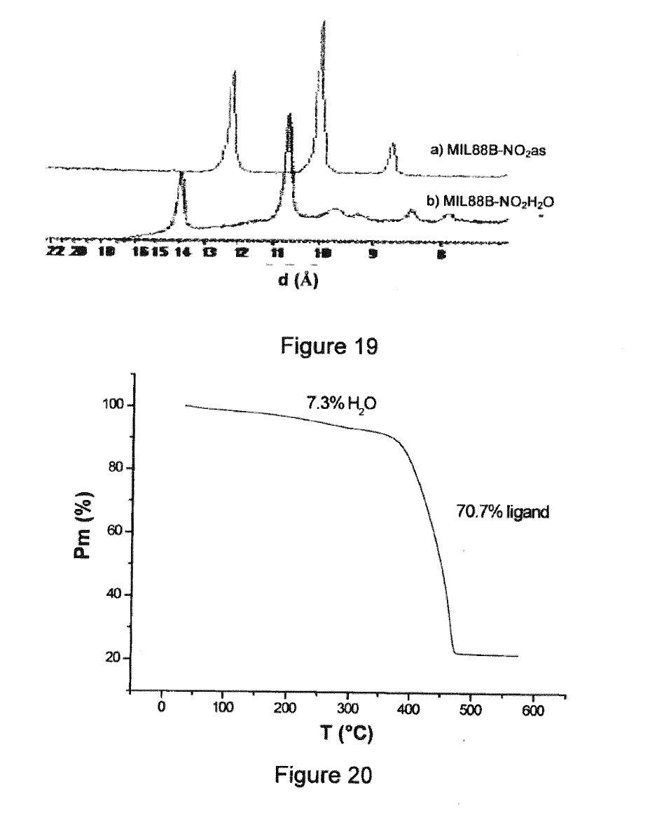

FIG. 19 shows the X-ray diffraction pattern of the solid MIL-88B-NO₂, raw (curve (a), top) and hydrated (curve (b), bottom).

The results of thermogravimetric analysis (in air, at a heating rate of 2° C./minute) of the compound MIL-88B-NO₂(Fe), after washing and drying, are shown in FIG. 20. The weight loss Pm (%) is shown as a function of the temperature T (in ° C.).

This compound does not have a surface accessible (greater than 20 m²/g) to nitrogen at 77 K, since the dry structure has a pore size that is too small to incorporate nitrogen N₂.

TABLE 7
elemental analysis

| Element | | | |
|---|---|---|---|
| (wt. %) | % Iron | % Carbon | % Nitrogen |
| MIL-88B-NO₂ | 20.6 | 39.3 | 4.6 |

i) MIL-88B-20H(Fe) or Fe₃O[C₆H₂(OH)₂(CO₂)₂]₃.X.nH₂O (X=F, Cl, OH)

Synthesis of the Solid MIL-88B-2OH(Fe):

354 mg (1 mmol) of Fe(ClO₄)₃.xH₂O (Aldrich, 99%) and 198 mg (1 mmol) of 2,5-dihydroxoterephthalic acid (obtained by hydrolysis of the corresponding diethyl ester, Aldrich 97%) are dispersed in 5 ml of DMF (Fluka, 98%). The mixture is left in a 23-ml Teflon container that is put in a PAAR metal bomb for 12 hours at 85° C. The solid is recovered by filtration.

To remove the acid remaining in the pores, the product is calcined at 150° C. under vacuum for 15 hours.

### Characteristic Data of the Solid MIL-88B-2OH(Fe):

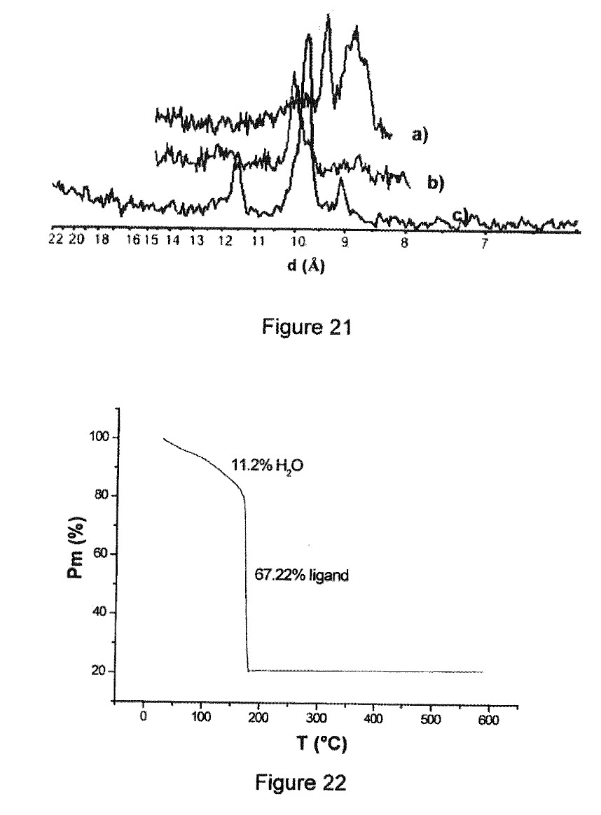

FIG. 21 shows the X-ray diffraction pattern of the solid MIL-883-2OH, raw (curve (c), bottom), hydrated (curve (b), middle) and dried under vacuum (curve (a), top).

The results of thermogravimetric analysis (in air, at a heating rate of 2° C./minute) of the compound MIL-88B-2OH(Fe), after washing and drying, are shown in FIG. 22. The weight loss Pm (%) is shown as a function of the temperature T (in ° C.).

This compound does not have a surface accessible (greater than 20 m²/g) to nitrogen at 77 K, since the dry structure has a pore size that is too small to incorporate nitrogen N₂.

TABLE 8
elemental analysis

| Element | | |
|---|---|---|
| (wt. %) | % Iron | % Carbon |
| MIL-88B-20H | 15.4 | 36.5 |

j) MIL-88B-NH₂ (Fe) or Fe₃O[C₆H₃NH₂—(CO₂)₂]₃.X.nH₂O (X=F, Cl, OH)

### Synthesis of the Solid MIL-88B-NH₂(Fe):

0.27 g (1 mmol) of FeCl₃.6H₂O (Alfa Aesar, 98%) and 180 mg (1 mmol) of 2-aminoterephthalic acid (Fluka, 98%) are dispersed in 5 ml of absolute ethanol. The mixture is left in a 23-ml Teflon container that is put in a PAAR metal bomb for 3 days at 100° C. The solid is recovered by filtration.

To remove the acid remaining in the pores, the solid is calcined at 200° C. for 2 days.

### Characteristic Data of the Solid MIL-88B-NH₂(Fe):

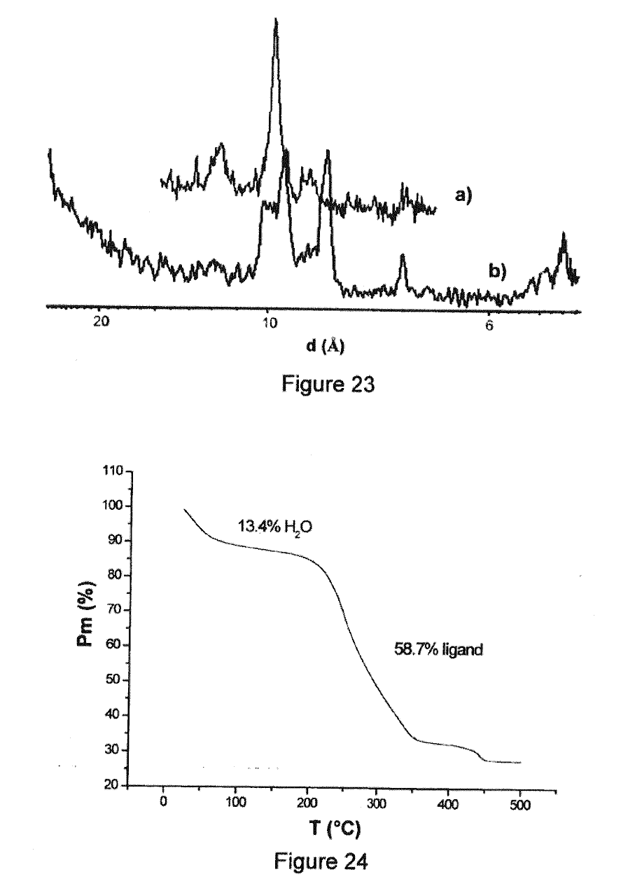

FIG. 23 shows the X-ray diffraction pattern of the solid MIL-88B-NH₂, raw (curve (b), bottom), and dried under vacuum (curve (a), top).

The results of thermogravimetric analysis (in air, at a heating rate of 2° C./minute) of the hydrated solid MIL-88B-NH₂(Fe) are shown in FIG. 24.

This compound does not have a surface accessible (greater than 20 m²/g) to nitrogen at 77 K, since the dry structure has a pore size that is too small to incorporate nitrogen N₂.

k) MIL-88B-Cl (Fe) or Fe₃O[C₆H₃Cl—(CO₂)₂]₃.X.nH₂O (X=F, Cl, OH)

### Synthesis of the Solid MIL-88B-Cl(Fe):

354 mg (1 mmol) of Fe(ClO₄)₃.xH₂O (Aldrich, 99%) and 200 mg (1 mmol) of 2-chloroterephthalic acid (synthesized according to synthesis A in example 3) are dispersed in 10 ml of DMF with 0.1 mL of 5M HF (SDS, 50%) and 0.1 mL of 1M HCl (Aldrich, 37%). The mixture is left in a 23-ml Teflon container that is put in a PAAR metal bomb for 5 days at 100° C. The solid is recovered by filtration.

The solid obtained is calcined at 150° C. under vacuum.

### Characteristic Data of the Solid MIL-88B-Cl (Fe):

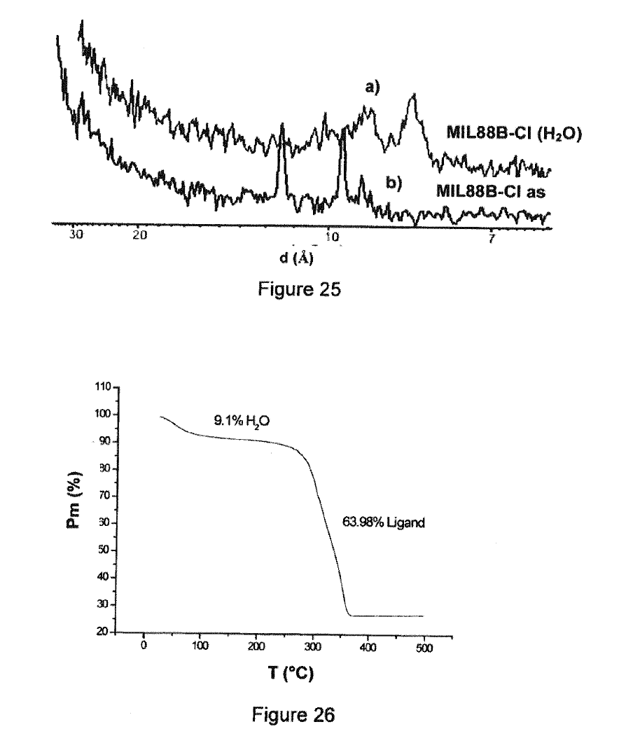

FIG. 25 shows the X-ray diffraction pattern of the solid MIL-88B-Cl, raw (curve (a), top) and hydrated (curve (b), middle) and solvated with DMF (curve (c), bottom).

The thermogravimetric analysis (in air, at a heating rate of 2° C./minute) of the hydrated solid MIL-88B-Cl(Fe) is shown in FIG. 26.

This compound does not have a surface accessible (greater than 20 m²/g) to nitrogen at 77 K, since the dry structure has a pore size that is too small to incorporate nitrogen N₂.

l) MIL-88B-4-CH₃ (Fe) or Fe₃O[C₆(CH₃)₄—(CO₂)₂]₃.X.nH₂O (X=F, Cl, OH)

### Synthesis of the Solid MIL-88B-4CH₃ (Fe):

0.27 g (1 mmol) of FeCl₃.6H₂O (Alfa Aesar, 98%), 222 mg (1 mmol) of 1,4-tetramethylterephthalic acid (Chem Service, 95%) are dispersed in 10 ml of DMF (Fluka, 98%) with 0.4 mL of 2M soda (Alfa Aesar, 98%). The mixture is left in a 23-ml Teflon container that is put in a PAAR metal bomb for 12 hours at 100° C. The solid is recovered by filtration.

200 mg of the solid is suspended in 100 mL of water with stirring at room temperature for 12 hours to remove the acid remaining in the pores. The solid is then recovered by filtration.

### Characteristic Data of the Solid MIL-88B-4CH₃ (Fe):

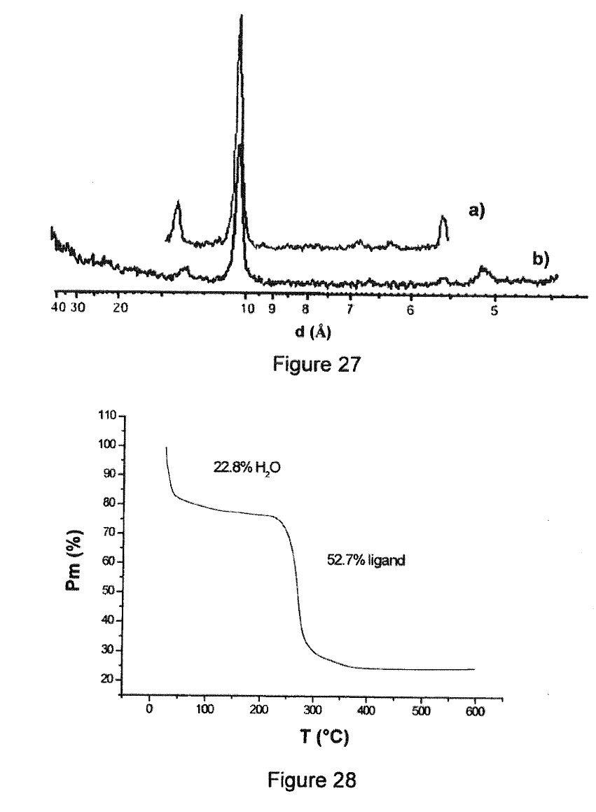

FIG. 27 shows the X-ray diffraction pattern of the raw solid (curve (b), bottom) and of the hydrated solid (curve (a), top).

The thermogravimetric analysis (in air, at a heating rate of 2° C./minute) of the hydrated solid MIL-88B-4CH₃(Fe) is shown in FIG. 28.

This compound has an accessible surface of the order of 1200 m²/g (Langmuir) to nitrogen at 77 K, since the dry structure possesses a sufficient pore size (6-7 Å) to incorporate nitrogen N₂.

m) MIL-88B-4F (Fe) or Fe₃O[C₆F₄—(CO₂)₂]₃.X.nH₂O (X=F, Cl, OH)

### Synthesis of the Solid MIL-88B-4F (Fe):

270 mg (1 mmol) of FeCl₃.6H₂O (Alfa Aesar, 98%) and 230 mg (1 mmol) of tetrafluoroterephthalic acid (Aldrich, 98%) are dispersed in 10 ml of distilled water. The mixture is left in a 23-ml Teflon container that is put in a PAAR metal bomb for 12 hours at 85° C. The solid is recovered by filtration.

200 mg of the solid is suspended in 20 mL of water with stirring at room temperature for 2 hours to remove the acid remaining in the pores. The solid is then recovered by filtration.

### Characteristic Data of the Solid MIL-88B-4F (Fe):

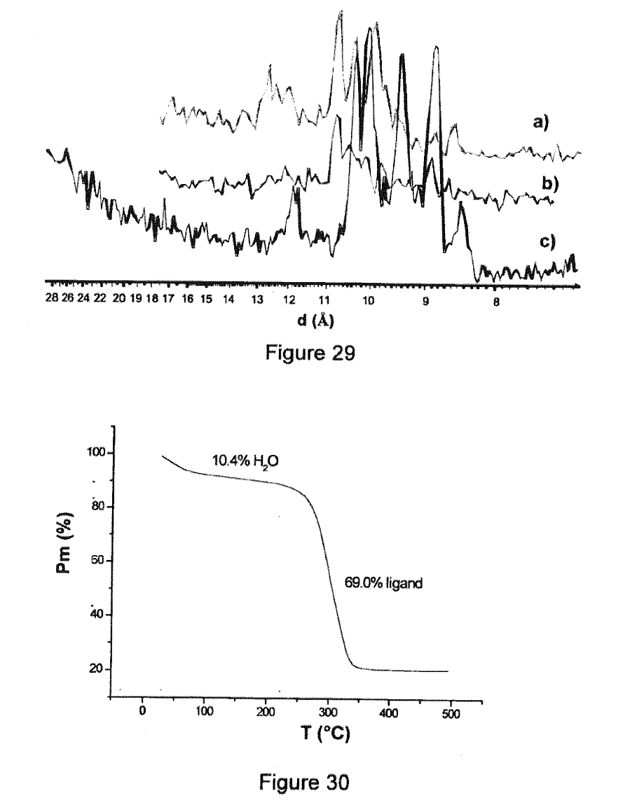

FIG. 29 shows the X-ray diffraction pattern of the raw solid (curve (c), bottom), of the hydrated solid (curve (b)) and of the solid solvated with ethanol (curve (a), top).

The thermogravimetric analysis (in air, at a heating rate of 2° C./minute) of the hydrated solid MIL-88B-4F(Fe) is shown in FIG. 30.

This compound does not have a surface accessible (greater than 20 m²/g) to nitrogen at 77 K, since the dry structure has a pore size that is too small to incorporate nitrogen N₂.

n) MIL-88B-Br (Fe) or Fe₃O[C₆H₃Br—(CO₂)₂]₃.X.nH₂O (X=F, Cl, OH)

### Synthesis of the Solid MIL-88B-Br (Fe):

270 mg (1 mmol) of FeCl₃. 6H₂O (Alfa Aesar, 98%) and 250 mg (1 mmol) of 2-bromoterephthalic acid (Fluka, 95%) are dispersed in 10 ml of DMF (Fluka, 98%) with 0.2 mL of 5M hydrofluoric acid (SDS, 50%). The mixture is left in a 23-ml Teflon container that is put in a PAAR metal bomb for 12 hours at 150° C. The solid is recovered by filtration.

To remove the acid remaining in the pores, the solid is calcined at 150° C. under vacuum for 15 hours.

### Characteristic Data of the Solid MIL-88B-Br (Fe):

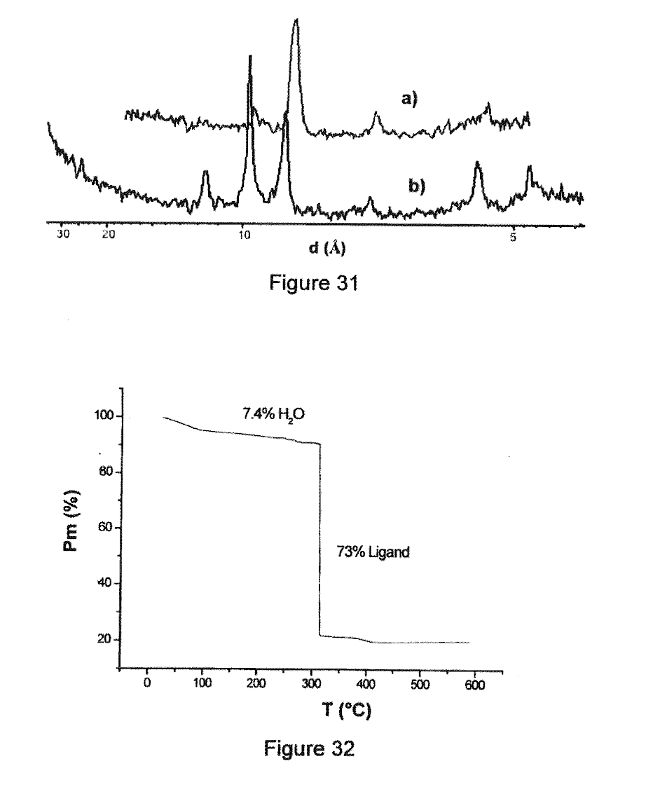

FIG. 31 shows the X-ray diffraction pattern of the raw solid (curve (b), bottom) and of the hydrated solid (curve (a), top).

The thermogravimetric analysis (in air, at a heating rate of 2° C./minute) of the hydrated solid MIL-88B-Br(Fe) is shown in FIG. 32.

This compound does not have a surface accessible (greater than 20 m²/g) to nitrogen at 77 K, since the dry structure has a pore size that is too small to incorporate nitrogen N₂.

o) MIL-88F (Thio) (Fe) or Fe₃O[C₄H₂S—(CO₂)₂]₃.X.nH₂O (X=F, Cl, OH)

### Synthesis of the Solid MIL-88F(Fe):

354 mg (1 mmol) of Fe(ClO₄)₃.xH₂O (Aldrich, 99%) and 258 mg (1 mmol) of 2,5-thiophenedicarboxylic acid (Aldrich, 99%) are dispersed in 2.5 ml of DMF (Fluka, 98%) with 0.1 mL of 5M HF (SDS, 50%). The mixture is left in a 23-ml Teflon container that is put in a PAAR metal bomb for 3 days at 100° C. The solid is recovered by filtration.

200 mg of the solid is suspended in 100 mL of water with stirring at room temperature for 12 hours to remove the acid remaining in the pores. The solid is then recovered by filtration.

### Characteristic Data of the Solid MIL-88F(Fe):

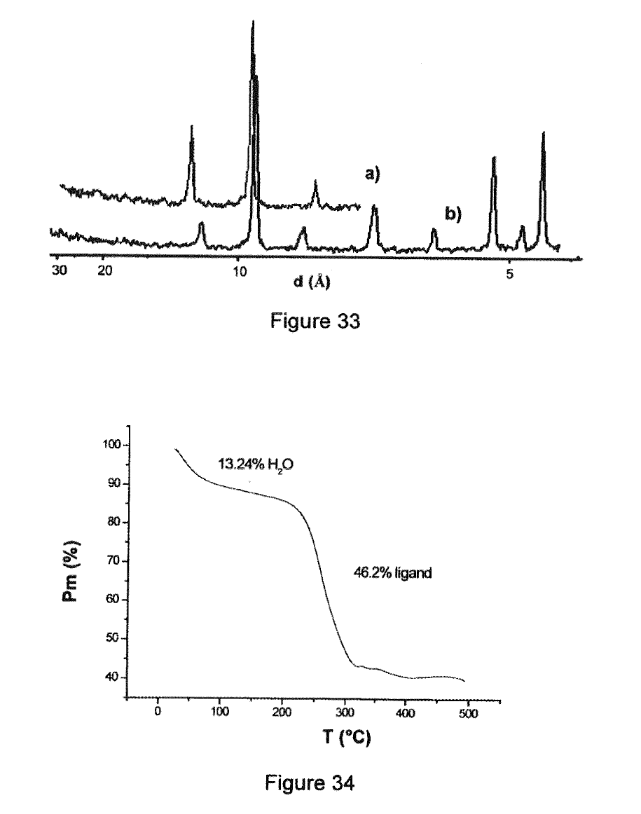

FIG. 33 shows the X-ray diffraction patterns of the raw solid (curve (b), bottom) and of the hydrated solid (curve (a), top).

The thermogravimetric analysis (in air, at a heating rate of 2° C./minute) of the hydrated solid MIL-88F(Fe) is shown in FIG. 34.

This compound does not have a surface accessible (greater than 20 m²/g) to nitrogen at 77 K, since the dry structure has a pore size that is too small to incorporate nitrogen N₂.

p) MIL-88G (AzBz) (Fe) or Fe₃O[C₁₂H₈N₂—(CO₂)₂]₃.X.nH₂O (X=F, Cl, OH)

### Synthesis of the Solid MIL-88G(Fe):

118 mg (0.33 mmol) of Fe(ClO₄)₃.xH₂O (Aldrich, 99%) and 90 mg (0.33 mmol) of 4,4′-azobenzenedicarboxylic acid (synthesized according to the method described by Ameerunisha et al., J. Chem. Soc. Perkin Trans. 2 1995, 1679 [88], are dispersed in 15 ml of DMF (Fluka, 98%). The mixture is left in a 23-ml Teflon container that is put in a PAAR metal bomb for 3 days at 150° C. The solid is recovered by filtration.

200 mg of the solid is suspended in 10 mL of DMF with stirring at room temperature for 2 hours to exchange the acid remaining in the pores. The solid is then recovered by filtration and the DMF remaining in the pores is removed by calcination at 150° C. under vacuum for 15 hours.

### Characteristic Data of the Solid MIL-88G(Fe):

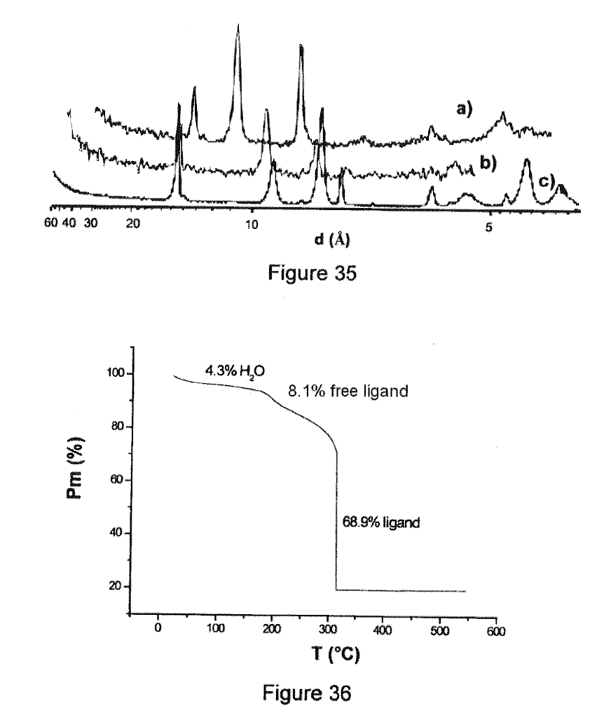

FIG. 35 shows the X-ray diffraction patterns of the solid MIL-88G, raw (curve (c), bottom), solid solvated with DMF (curve (b), middle) and solid solvated with pyridine (curve (a), top).

The thermogravimetric analysis (in air, at a heating rate of 2° C./minute) of the raw solid MIL-88G(Fe) is shown in FIG. 36.

This compound does not have a surface accessible (greater than 20 m²/g) to nitrogen at 77 K, since the dry structure has a pore size that is too small to incorporate nitrogen N₂.

q) MIL-88G-2Cl (AzBz-2Cl) (Fe) or Fe₃O[C₁₂H₆N₂Cl₂—(CO₂)₂]₃.X.nH₂O (X=F, Cl, OH)

### Synthesis of the Solid MIL-88G-2Cl (Fe):

177 mg (0.5 mmol) of Fe(ClO₄)₃.xH₂O (Aldrich, 99%) and 169 mg (0.5 mmol) of dichloro-4,4′-azobenzenedicarboxylic acid (synthesized according to synthesis D described in example 3) are dispersed in 15 ml of DMF (Fluka, 98%). The mixture is left in a 23-ml Teflon container that is put in a PAAR metal bomb for 12 hours at 150° C. The solid is recovered by filtration.

200 mg of the solid is suspended in 10 mL of DMF with stirring at room temperature for 2 hours to exchange the acid remaining in the pores. The solid is then recovered by filtration, and the DMF remaining in the pores is removed by calcination at 150° C. under vacuum for 15 hours.

Characteristic data of the solid MIL-88G-2Cl (Fe):

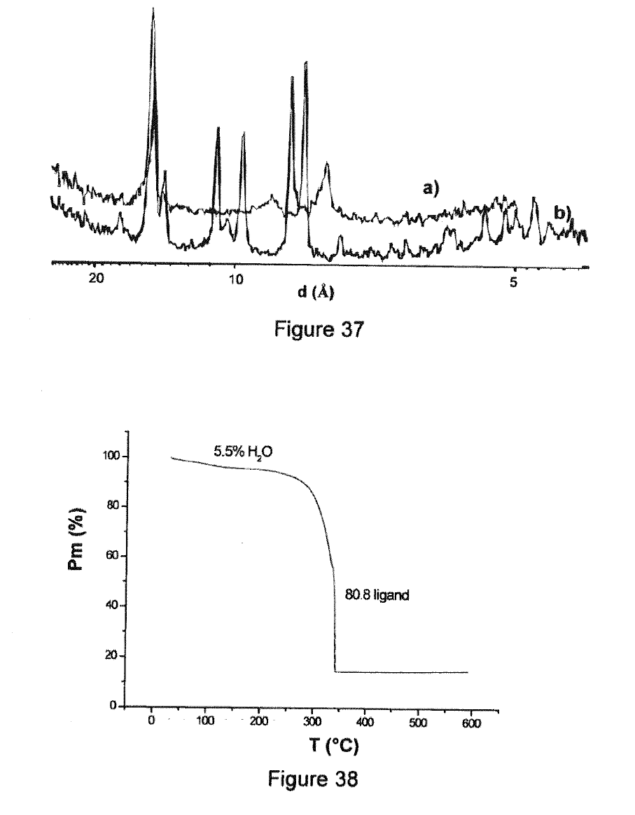

FIG. 37 shows the X-ray diffraction patterns of the raw solid MIL-88G-2Cl (curve (b), bottom) and of the dry solid MIL-88G-2Cl (curve (a), top).

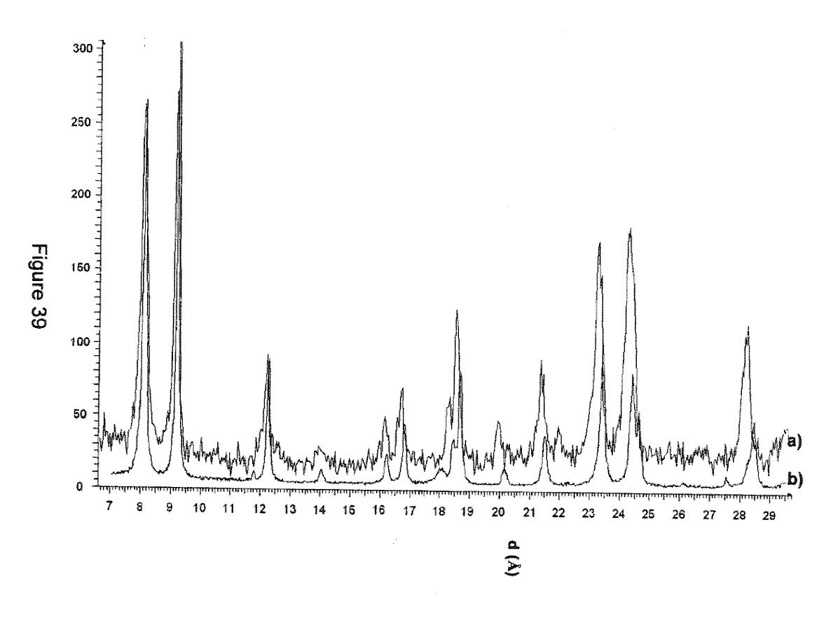

The thermogravimetric analysis (in air, at a heating rate of 2° C./minute) of the raw solid MIL-88G-2Cl (Fe) is shown in FIG. 38.

This compound does not have a surface accessible (greater than 20 m²/g) to nitrogen at 77 K, since the dry structure has a pore size that is too small to incorporate nitrogen N₂.

r) MIL-102(Fe) or Fe₆O₂X₂[C₁₀H₂—(CO₂)₄]₃.nH₂O (X=F, Cl etc.)

### Synthesis of the Solid MIL-102(Fe):

270 mg (1 mmol) of FeCl₃.6H₂O (Alfa Aesar, 98%) and 268 mg (1 mmol) of 1,4,5,8-naphthalenetetracarboxylic acid are dispersed in 5 ml of distilled water. The mixture is left in a 23-ml Teflon container that is put in a PAAR metal bomb for 15 hours at 100° C. The solid is recovered by filtration.

### Characteristic Data of the Solid MIL-102(Fe):

FIG. 39 shows the X-ray diffraction patterns of the raw solid MIL-102(Fe) (curve (a)) and of the solid MIL-102(Cr) (curve (b)).

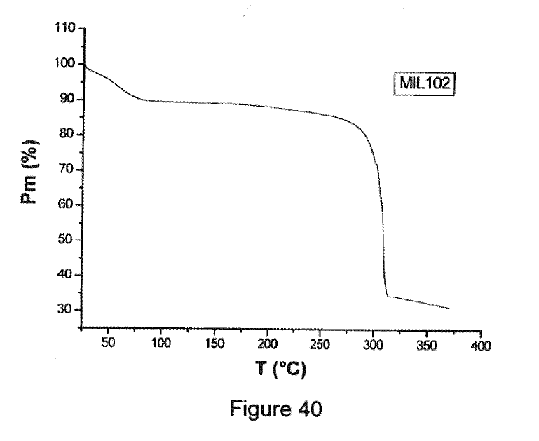

The thermogravimetric analysis (in air, at a heating rate of 2° C./rain) of the raw solid MIL-102(Fe) is shown in FIG. 40.

This compound has a small specific surface (Langmuir surface: 101 m²/g) to nitrogen at 77 K.

s) MIL-126(Fe) or Fe₆O₂X₂[C₁₀H₂—(CO₂)₄]₃.nH₂O (X=F, Cl etc.)

### Synthesis of the Solid MIL-126(Fe):

270 mg (1 mmol) of FeCl₃.6H₂O (Alfa Aesar, 98%) and 140 mg (0.6 mmol) of 4,4′-biphenyldicarboxylic acid (Fluka, 95%) are dispersed in 5 ml of dimethylformamide (DMF, Aldrich, 99%). The mixture is left in a 23-ml Teflon container that is put in a PAAR metal bomb for 12 hours at 150° C. with a plateau of temperature increase of 1 hour and a plateau of temperature decrease of 1 hour. The solid is recovered by filtration.

The solid is then dried at 150° C. under primary vacuum for 15 hours.

### Characteristic Data of the Solid MIL-126(Fe):

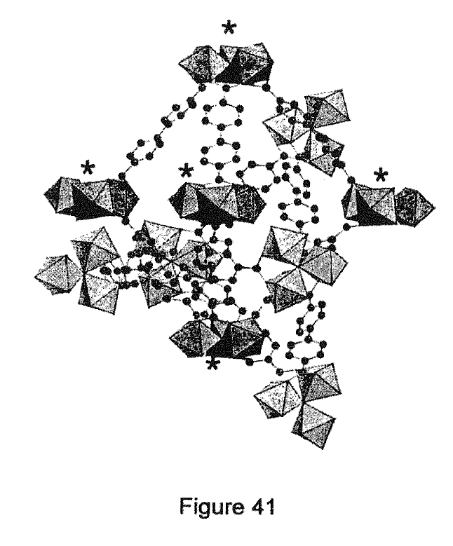

The crystallographic structure of the solid MIL-126(Fe) is an interpenetrated form of the structure MIL-88D(Fe), i.e. it possesses two enmeshed sublattices of type MIL-88D (FIG. 41).

Analysis of the crystal structure of the solid gives the characteristics presented in the following table:

TABLE 9
lattice parameters of the solid MIL-126,
dry and solvated (dimethylformamide)

| | | | Unit cell | | |
|---|---|---|---|---|---|
| | a | c | volume | Pore size | Space |
| Phase | (Å) | (Å) | (Å³) | (Å) | group |
| MIL-126 dry | 19.5 | 35.3 | 13500 | 4 to 10 | P 41 21 2 |
| MIL-126 | 21.8 | 36.1 | 17200 | 5 to 11 | P 41 21 2 |
| solvated | | | | | |
| (DMF) | | | | | |

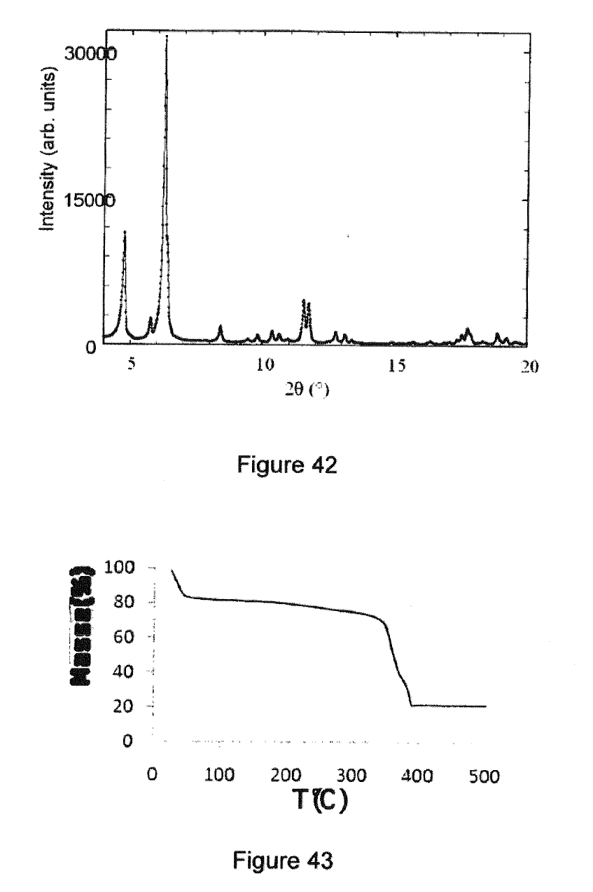

FIG. 42 shows the X-ray diffraction pattern of the raw solid MIL-126(Fe) (lambda_{Cu}=1.5406 angstrom).

The results of thermogravimetric analysis of the compound MIL-126(Fe), raw from synthesis (in air, at a heating rate of 2° C./minute) are shown in FIG. 43 (weight loss Pm as a function of the temperature T in ° C.).

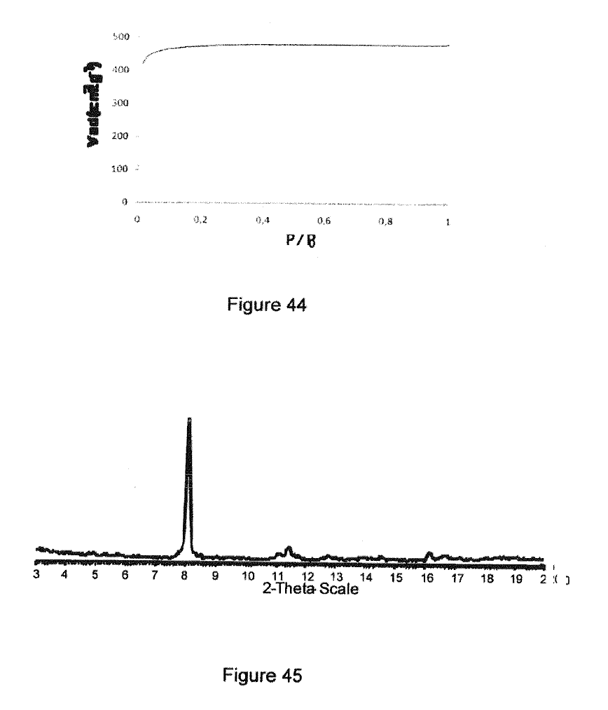

This compound has a large accessible surface (Langmuir) (greater than 2100 m²/g) to nitrogen at 77 K (FIG. 44). FIG. 44 is a graph of a nitrogen adsorption isotherm of MIL-126(Fe) (P₀=1 atmosphere).

t) MIL-127 (Fe) or Fe₆O₂ C₁₂H₆N₂—(CO₂)₄].X₂.nH₂O (X=F, Cl, OH)

The phase that is isotypical of that with indium published by Y. Liu et al., Angew. Chem. Int. Ed. 2007, 46, 3278-3283 [89] is prepared in this example.

### Synthesis of the Solid MIL-127(Fe):

The solid is recovered by filtration and dried under vacuum at 90° C.

118 mg (0.3 mmol) of Fe(ClO₄)₃.nH₂O (Aldrich, 98%) and 119 mg (0.6 mmol) of 3,3′,5,5′-azobenzenetetracarboxylic acid (synthesized according to synthesis protocol E described in example 3 below) are dispersed in 5 mL of dimethylformamide (DMF, Aldrich, 99%) with addition of 0.1 mL of 5M hydrofluoric acid (HF, SDS 50%). The mixture is left in a 23-ml Teflon container that is put in a PAAR metal bomb for 3 days at 150° C. with a plateau of temperature increase of 1 hour. The solid is recovered by filtration.

The solid is then dried at 200° C. under primary vacuum for 15 hours.

Characteristic data of the solid MIL-127(Fe): iron 3,3′,5,5′-azobenzenetetracarboxylate

FIG. 45 shows the X-ray diffraction pattern of the solid iron(III) 3,3′,5,5′-azobenzenetetracarboxylate, raw from synthesis.

The phase, with cubic symmetry, is isostructural with that published by the group of Prof. Eddaoudi [89] with indium (space group Pa3).

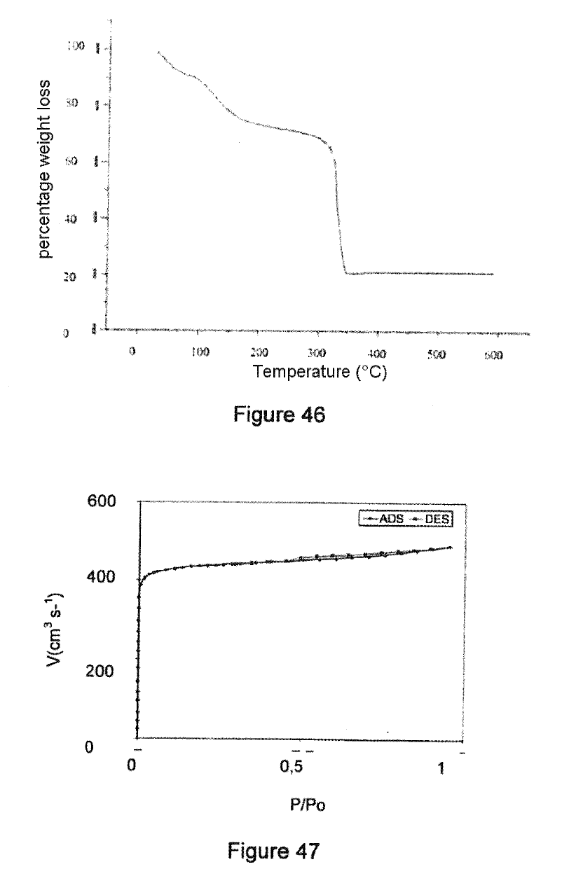

The results of thermogravimetric analysis of the compound iron 3,3′,5,5′-azobenzenetetracarboxylate, raw from synthesis (in air, at a heating rate of 2° C./minute) are shown in FIG. 46 (weight loss Pm as a function of temperature T).

The weight losses observed at temperatures below 250° C. correspond to the solvent (water, dimethylformamide) present in the pores.

The product decomposes at around 300° C., giving the iron(III) oxide.

This compound has a large accessible surface (Langmuir) (greater than 2000 m²/g) to nitrogen at 77 K (FIG. 47) (nitrogen porosimetry, Micromeritics ASAP 2010 instrument). FIG. 47 shows a nitrogen adsorption isotherm of iron 3,3′,5,5′-azobenzenetetracarboxylate (P₀=1 atmosphere).

u) MIL-88B-4CH₃ (Fe) or Fe₃O[C₆(CH₃)₄—(CO₂)₂]₃.X.nH₂O (X=F, Cl, OH)

The synthesis conditions are as follows: 0.27 g of FeCl₃.6H₂O (1 mmol, Alfa Aesar, 98%), 222 mg (1 mmol) of 1,4-tetramethylterephthalic acid (Chem Service, 95%) dispersed in 10 ml of DMF (Fluka, 98%) with 0.4 mL of 2M NaOH (Alfa Aesar, 98%), the whole left in a 23-ml Teflon container that is put in a PAAR metal bomb for 12 hours at 100° C.

The solid is recovered by filtration.

200 mg of the solid is suspended in 100 mL of water with stirring at room temperature for 12 hours to remove the acid remaining in the pores. Then the solid is recovered by filtration.

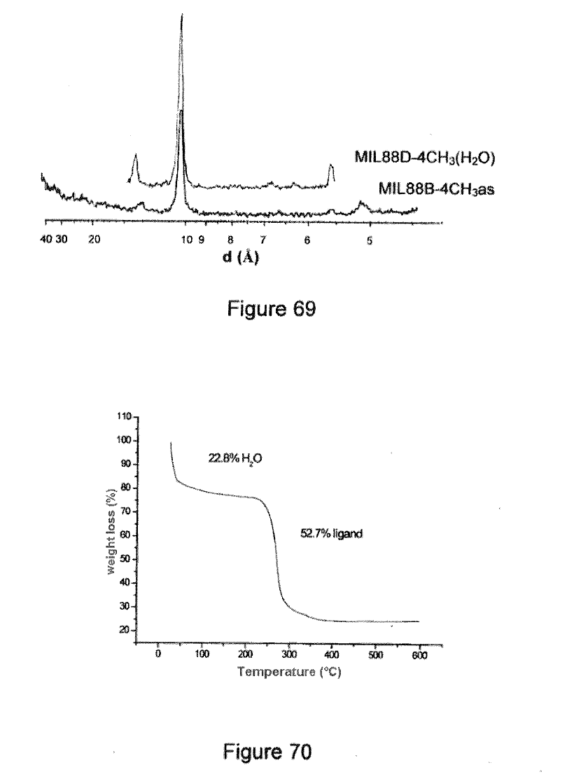

FIG. 69 shows an X-ray diffraction pattern of the raw solid MIL-88B 4-CH₃ obtained (lower curve) and hydrated (upper curve).

FIG. 70 shows a thermogravimetric analysis in air of the hydrated compound MIL-88B 4-CH₃(Fe) obtained with a heating rate of 2° C./minute).

v) MIL-88D 4-CH₃ (Fe) or Fe₃O(C₁₂H₄ (CH₃)₄—(CO₂)₂]₃.X.nH₂O (X=F, Cl, OH)

The synthesis conditions are as follows: 354 mg of Fe(ClO₄)₃.xH₂O (1 mmol, Aldrich, 99%), 298 mg (1 mmol) of tetramethylbiphenyl-4,4′-dicarboxylic acid (synthesized according to synthesis protocol B described in example 3 below) dispersed in 5 ml of DMF (Fluka, 98%) with 0.2 mL of 2M NaOH (Alfa Aesar, 98%), the whole left in a 23-ml Teflon container that is put in a PAAR metal bomb for 12 hours at 100° C.

The solid is recovered by filtration.

200 mg of the solid is suspended in 10 mL of DMF with stirring at room temperature for 2 hours to exchange the acid remaining in the pores of the solid. After this, the solid is recovered by filtration. To remove the DMF remaining in the pores, the solid is calcined at 150° C. under vacuum for 15 hours.

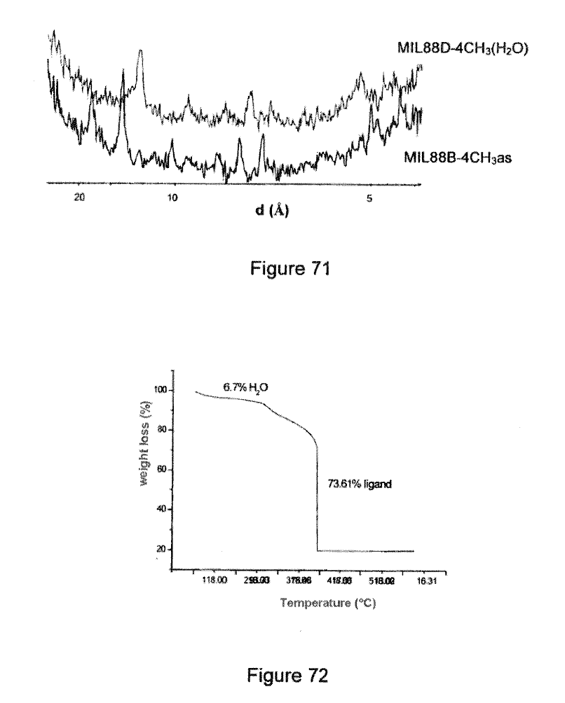

FIG. 71 shows an X-ray diffraction pattern of the raw solid MIL-88D 4-CH₃ obtained (lower curve) and hydrated (upper curve).

FIG. 72 shows a thermogravimetric analysis in air of the hydrated compound MIL-88D 4-CH₃(Fe) obtained with a heating rate of 2° C./minute).

w) MIL-88D 2CH₃ (Fe) or Fe₃O[C₁₂H₆ (CH₃)₂—(CO₂)₂]₃.X.nH₂O (X=F, Cl, OH)

The synthesis conditions are as follows: 270 mg of FeCl₃.6H₂O (1 mmol, Alfa Aesar, 98%), 268 mg (1 mmol) of dimethylbiphenyl-4,4′-dicarboxylic acid (synthesized according to synthesis protocol C of example 3 below) dispersed in 5 mL of DMF (Fluka, 98%) with 0.25 mL of 5M HF (SDS, 50%), the whole left in a 23-ml Teflon container that is put in a PAAR metal bomb for 12 hours at 150° C.

The solid is recovered by filtration.

To remove the acid remaining in the pores, the solid is calcined at 150° C. under vacuum for 15 hours.

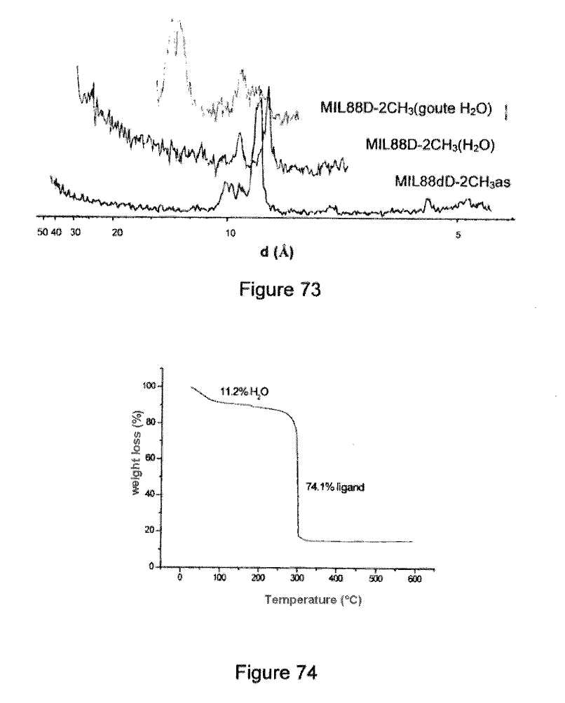

FIG. 73 shows an X-ray diffraction pattern of the raw solid MIL-88D 2CH₃ (lower curve), hydrated (middle curve) and wetted with excess water (upper curve).

FIG. 74 shows a thermogravimetric analysis in air of the hydrated compound MIL-88D 2CH₃ (Fe) obtained with a heating rate of 2° C./minute.

### Example 2

### Synthesis of Iron(III) Acetate for Manufacture of MOFs Usable in the Present Invention

The iron(III) acetate used in the examples given below for synthesis of the MOF materials according to the invention is synthesized according to the following protocol. This synthesis can refer to the work of Dziobkowski et al., Inorg. Chem., 1982, 21, 671 [87].

6.72 g of metallic iron powder (Riedel-de Haën, 99%), 64 mL of deionized water and 33.6 mL of perchloric acid at 70% in water (Riedel-de Haën) are mixed with magnetic stirring and heated at 50° C. for 3 hours. After switching off the heating, the solution is stirred for 12 hours. The residual metallic iron is removed by decanting followed by a change of container. 20.6 mL of hydrogen peroxide solution in water (marketed by the company Alfa Aesar, 35%) is added dropwise with stirring, the whole being kept in an ice bath at 0° C. 19.7 g of sodium acetate (Aldrich, 99%) is added to the blue solution with stirring, maintaining the solution at 0-5° C. The solution is left to evaporate for 3 days under a hood in a glass crystallizing dish (volume=0.5 L). Finally, the red crystals of iron acetate are recovered by filtration and are washed very quickly with iced deionized water. The crystals are then dried in air.

### Example 3

### Synthesis of the Ligands of the MOFs Usable in the Present Invention

### a) Synthesis A: Synthesis of Chloroterephthalic Acid

6 g (0.043 mol) of chloroxylene (marketed by the company Aldrich, >99%), 16 mL of nitric acid (marketed by the company VWR, 70%) and 60 mL of distilled water are put in a 120-mL Teflon container. The latter is put in a PAAR metal bomb, and heated at 170° C. for 12 h. The product is recovered by filtration, then washed with copious amounts of distilled water. A yield of 75% is obtained.

¹H NMR (300 MHz, d6-DMSO): δ (ppm): 7.86 (d, J=7.8 Hz), 7.93 (dd, J=7.8; 1.2 Hz), 7.96 (d, J=1.2 Hz)

### b) Synthesis B: synthesis of 3,5,3′,5′-tetramethylbiphenyl-4,4′-dicarboxylic acid

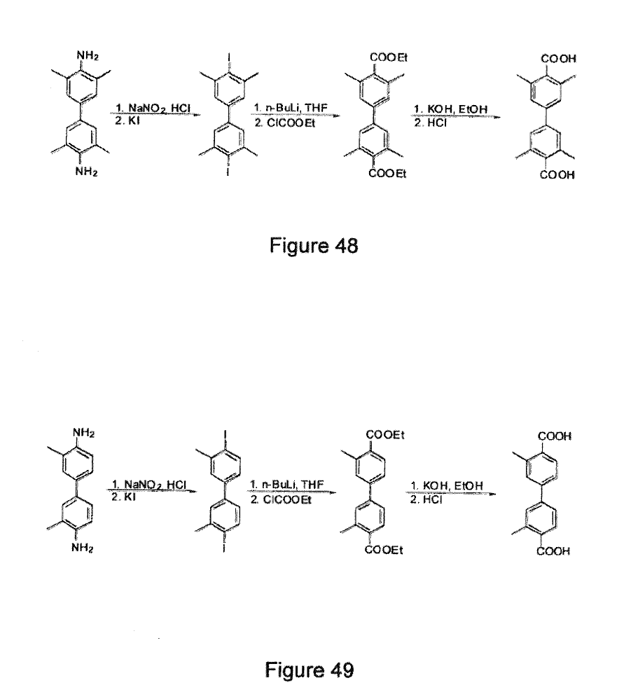

The reaction scheme of this synthesis is shown in FIG. 48.

### 1st Step:

10.2 g of tetramethylbenzidine (98%, Alfa Aesar) is suspended in 39 mL of concentrated hydrochloric acid (37%, marketed by the company Aldrich) at 0° C. Diazotization was carried out by adding a solution of sodium nitrite (6 g in 50 mL of water). After stirring for 15 min at 0° C., a solution of potassium iodide (70 g in 200 mL of water) was added slowly to the resultant violet solution. On completion of addition, the mixture is stirred for 2 hours at room temperature. The resultant black suspension is filtered, recovering a black precipitate, which is washed with water. The solid is suspended in dichloromethane (DCM, 98%, marketed by the company SDS) and a saturated solution of sodium thiosulfate is added, causing bleaching. After stirring for 1 hour, the organic phase is decanted and the aqueous phase is extracted with DCM. The organic phase is dried over sodium sulfate, then evaporated to give the diiodinated intermediate in the form of a greyish solid. Elution with pure pentane on a silica column (marketed by the company SDS) makes it possible to obtain the mixture of monoiodinated and diiodinated compounds. The mixture of the latter was used directly in the next step.

### 2nd Step:

7.2 g of the raw iodinated compound is dissolved in 100 mL of tetrahydrofuran (THF, distilled over sodium). After cooling to −78° C., 35 mL of n-butyllithium in cyclohexane (2.5 M, marketed by the company Aldrich) is added. The solution is allowed to return to room temperature; a white suspension appears after 2 hours. It is cooled again to −78° C. and 12 mL of ethylchloroformate is added. The mixture is left at room temperature; a clear yellow solution is obtained after 1 hour. Separation of water and dichloromethane, followed by extraction with dichloromethane gives the raw diester. The latter is purified by silica gel chromatography, with Et₂O/pentane:1/9 mixture as eluent (retardation factor: R_{f}=0.3). 6.3 g of diester is obtained in the form of a colorless solid (yield of 42% from benzidine).

Characterization of the diester obtained: ¹H NMR (300 MHz, CDCl₂): δ (ppm): 1.29 (t, J=7.2 Hz, 6H), 2.29 (s, 13H); 4.31 (q, J=7.2 Hz, 4H); 7.12 (s, 4H). ¹³C NMR (75 MHz, CDCl₃): δ (ppm): 14.3 (CH₃), 19.9 (CH₂), 61.0 (CH₂), 126.5 (CH), 133.2 (Cq), 135.5 (Cq), 141.4 (Cq), 169.8 (Cq).

### 3rd Step:

Finally, the diester is saponified with 9.7 g of potassium hydroxide (marketed by the company VWR) in 100 mL of 95% ethanol (marketed by the company SDS), under reflux for 5 days. The solution is concentrated under vacuum and the product is dissolved in water. Concentrated hydrochloric acid is added until pH 1, and a white precipitate is formed. It is recovered by filtration, washed with water and dried. 5.3 g of diacid is thus obtained in the form of a white solid (quantitative yield).

### c) Synthesis C: synthesis of 3,3′-dimethylbiphenyl-4,4′-dicarboxylic acid

The reaction scheme of this synthesis is shown in FIG. 49.

The same procedure as that described for synthesis B was used, starting from 12.1 g of dimethylbenzidine. At the end of the 1st step, 18.4 g of 3,3′-dimethyl-4,4′-diiodo-biphenyl is obtained (yield: 74%).

Characterization of the diiodinated compound obtained:

¹H NMR (300 MHz, CDCl₃): δ (ppm): 2.54 (s, 6H), 7.10 (dd, J=2.2 and 8.1 Hz, 2H), 7.46 (d, J=2.2 Hz, 2H), 7.90 (d, J=8.1 Hz, 2H). ¹³C NMR (75 MHz, CDCl₃): δ (ppm): 28.3 (CH₃), 100.3 (Cq), 126.0 (CH), 128.3 (CH), 139.4 (CH), 140.4 (Cq), 141.9 (Cq).

After the 2nd and 3rd steps, 6.9 g of 3,3′-dimethyl-biphenyl-4,4′-dicarboxylic acid is obtained from 18.4 g of diiodinated compound.

### Characterization of the Compounds Obtained:

The diester obtained after the 2nd step and the diacid obtained after the 3rd step have spectroscopic signatures identical to those described in the literature, for example in Shiotani Akinori, Z. Naturforsch. 1994, 49, 12, 1731-1736 [90].

### d) Synthesis D: synthesis of 3,3′-dichloro-4,4′-azobenzenedicarboxylic acid

15 g of o-chlorobenzoic acid (marketed by the company Aldrich, 98%) and 50 g of soda are put in 225 mL of distilled water, and heated at 50° C. with stirring. 100 g of glucose (Aldrich, 96%) dissolved in 150 mL of water is added. The mixture is stirred for 15 minutes, then it is bubbled with air for 3 hours, at room temperature. The disodium salt is recovered by filtration, washed with ethanol, then redissolved in 120 mL of water. Hydrochloric acid (marketed by the company Aldrich VWR, 37%) is added until the pH is equal to 1. The solid is recovered by filtration and dried under vacuum at 90° C.

### e) Synthesis E of 3,3′,5,5′-azobenzenetetracarboxylic acid

15 g of 2-nitro-isophthalic acid (marketed by the company Aldrich, 98%) and 50 g of soda are put in 225 mL of distilled water, and heated at 50° C. with stirring. 100 g of glucose (Aldrich, 96%) dissolved in 150 mL of water is added. The mixture is stirred for 15 minutes, then it is bubbled with air for 3 hours, at room temperature. The disodium salt is recovered by filtration, washed with ethanol, then redissolved in 120 mL of water. Hydrochloric acid (marketed by the company Aldrich VWR, 37%) is added until the pH is equal to 1.

### Example 4

### Synthesis of MOF Nanoparticles Usable in the Present Invention

### a) MIL-89 Nano

MIL-89 nano is synthesized from iron acetate (1 mmol; synthesized according to the synthesis described in example 2) and muconic acid (1 mmol; Fluka, 97%) in 5 mL of ethanol (Riedel-de Haën, 99.8%) with addition of 0.25 mL of 2M sodium hydroxide (Alfa Aesar, 98%) in an autoclave (Paar bomb) at 100° C. for 12 h. After cooling the container, the product is recovered by centrifugation at 5000 rpm (revolutions per minute) for 10 minutes.

200 mg of the solid is suspended in 100 mL of distilled water with stirring for 15 h to remove the solvent remaining in the pores. Then the solid is recovered by centrifugation at 5000 rpm for 10 minutes.

The particle size measured by light scattering is 400 nm (nanometers).

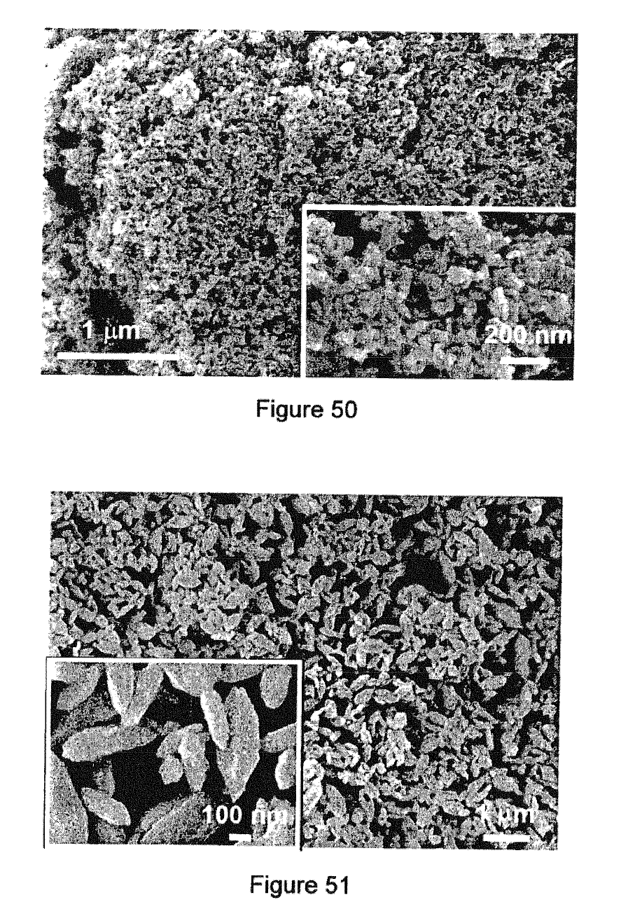

FIG. 50 shows a micrograph obtained by scanning electron microscopy (SEM) of the solid MIL-89 nano.

The nanoparticles show a rounded and slightly elongated morphology, with a very uniform particle size of 50-100 nm (FIG. 51).

### b) MIL-88Anano

To obtain the material MIL-88Anano, FeCl₃.6H₂O (1 mmol; Alfa Aesar, 98%) and fumaric acid (1 mmol; Acros, 99%) are dispersed in 15 mL of ethanol (Riedel-de Haën, 99.8%). Then 1 mL of acetic acid (Aldrich, 99.7%) is added to this solution. The solution is put in a glass bottle and heated at 65° C. for 2 hours. The solid is recovered by centrifugation at 5000 rpm for 10 minutes.

200 mg of the solid is suspended in 100 mL of distilled water with stirring for 15 h to remove the solvent remaining in the pores. Then the solid is recovered by centrifugation at 5000 rpm for 10 minutes.

The particle size measured by light scattering is 250 nm.

Scanning electron microscopy (SEM) of the solid MIL-88Anano is shown in FIG. 51. The SEM images (FIG. 51) show elongated particles with edges. There are two particle sizes: about 500 nm and 150 nm.

### c) MIL-100 Nano

MIL-100 nano is synthesized by mixing FeCl₃.6H₂O (1 mmol; Alfa Aesar, 98%) and 1,3,5-benzenetricarboxylic acid (1,3,5-BTC; 1 mmol; Aldrich, 95%) in 3 mL of distilled water. The mixture is put in a PAAR bomb at 100° C. for 12 h. The product is recovered by centrifugation at 5000 rpm (10 minutes).

200 mg of the solid is suspended in 100 mL of distilled water with stirring and reflux for 3 h to remove the acid remaining in the pores. Then the solid is recovered by centrifugation at 5000 rpm (10 minutes). The particle size measured by light scattering is 536 nm.

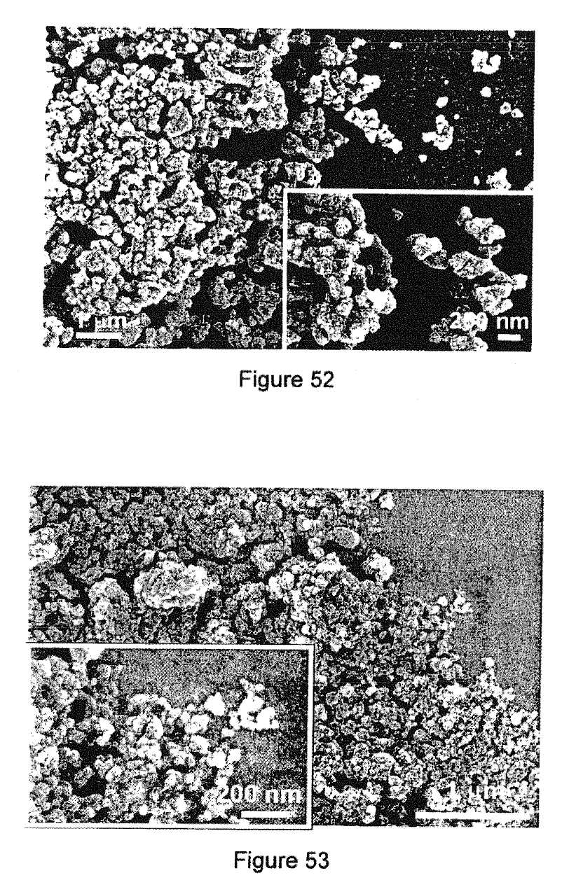

Scanning electron microscopy (SEM) of the solid MIL-100 nano is shown in FIG. 52.

A large particle cluster can be seen in the SEM images (FIG. 52). The nanoparticles are rather spherical, but the size is difficult to determine on account of the large cluster. A size of 40-600 nm can be estimated.

d) MIL-101 nano

To obtain the solid MIL-101 nano, a solution of FeCl₃.6H₂O (1 mmol; Alfa Aesar, 98%) and 1,4-benzenedicarboxylic acid (1.5 mmol; 1,4-BDC Aldrich, 98%) in 10 mL of dimethylformamide (Fluka, 98%) is put in a PAAR bomb and heated at 100° C. for 15 hours. The solid is recovered by centrifugation at 5000 rpm (10 minutes).

To remove the acid remaining in the pores, the product is heated at 200° C. under vacuum for 1 day. The product is stored under vacuum or inert atmosphere on account of its low stability in air.

The particle size measured by light scattering is 310 nm.

### e) MIL-88Btnano

The solid MIL-88Btnano is synthesized from a solution of FeCl₃.6H₂O (1 mmol; Alfa Aesar, 98%) and 1,4-benzenetetramethyldicarboxylic acid (1 mmol; Chem Service) in 10 mL of dimethylformamide (Fluka, 98%) with 0.4 mL of 2M NaOH. This solution is put in a PAAR bomb and heated at 100° C. for 2 hours. Then the container is cooled with cold water, and the product is recovered by centrifugation at 5000 rpm (10 minutes) (rpm=revolutions per minute).

200 mg of the solid is suspended in 100 mL of distilled water with stirring for 15 h to remove the solvent remaining in the pores. The solid is then recovered by centrifugation at 5000 rpm (10 minutes).

Measurement of particle size by light scattering shows two populations of nanoparticles of 50 and 140 nm.

The nanoparticles of the solid MIL-88Btnano have a spherical morphology with a size of about 50 nm. Only a minor fraction has a size of about 200 nm. Clusters of small particles can also be observed.

Scanning electron microscopy (SEM) of the solid MIL-88Btnano is shown in FIG. 53.

### f) MIL-88Bnano

The solid MIL-88Bnano is synthesized from a solution of iron acetate (1 mmol; synthesized according to the synthesis described in example 2) and 1,4-benzenedicarboxylic acid (1 mmol; 1,4-BDC Aldrich, 98%) in 5 mL of methanol (Aldrich, 99%). This solution is put in a PAAR bomb and heated at 100° C. for 2 hours. The container is then cooled with cold water, and the product is recovered by centrifugation at 5000 rpm (10 minutes).

200 mg of the solid is suspended in 100 mL of distilled water with stirring under reflux for 15 h to exchange the solvent remaining in the pores. Then the solid is recovered by centrifugation at 5000 rpm (10 minutes).

Measurement of particle size by light scattering shows a bimodal distribution of nanoparticles of 156 and 498 nm.

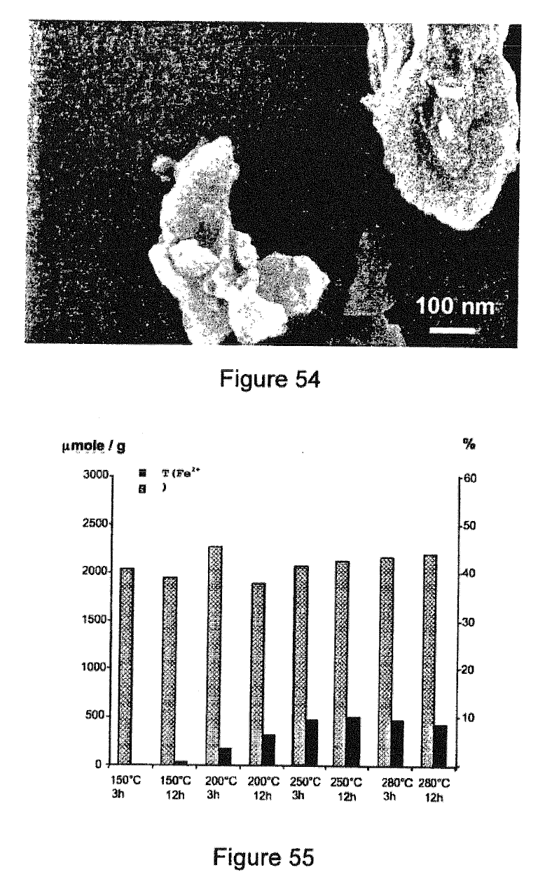

Scanning electron microscopy (SEM) of the solid MIL-88Bnano is shown in FIG. 54.

The morphology of the particles is very irregular, with a size of 300 nm.

Determination of particle size by light scattering was carried out on a Malvern Zetasizer Nano series -Nano-ZS instrument (model Zen 3600; serial No. 500180; UK).

Scanning electron microscopy was performed using a Topcon microscope (Akashi) EM 002B ultra-high resolution 200 kV.

The differences between the values supplied by the two techniques are due on the one hand to the orange coloration of the particles of iron carboxylates, the laser beam of the light scattering apparatus being red, and on the other hand to phenomena of particle clustering.

g) MIL-101-Cl (Fe) or Fe₃O[Cl—C₆H₃—(CO₂)₂]₃.X.nH₂O (X=F, Cl, OH)

The synthesis conditions are as follows: 0.27 g (1 mmol) of FeCl₃.6H₂O and 210 mg of chloro-1,4-benzenedicarboxylic acid (1.0 mmol, Cl-1,4-BDC, synthesized according to synthesis H described in example 1) are dispersed in 10 ml of DMF (dimethylformamide, Fluka, 98%). The whole is left for 12 h (hours) at 100° C. in a 23-ml Teflon container that is put in a PAAR metal bomb. The solid is then filtered and washed with acetone.

Lattice parameters of the solid MIL-101(Fe) at 298 K: a=89.0 Å and V=707000 A³, space group Fd-3m (No. 227)

The size of the monodispersed particles (polydispersity index, PDI=0.225) measured by light scattering is 400 nm.

h) MIL-101-NH₂(Fe) or Fe₃O[NH₂—C₆H₃—(CO₂)₂]₃.X.nH₂O (X=F, Cl, OH)

2.25 g (0.92 mmol) of FeCl₃.6H₂O and 0.75 mg of amino-1,4-benzenedicarboxylic acid (0.41 mmol, NH₂-1,4-BDC, Aldrich 99%) are dispersed in 50 mL of DMF (dimethylformamide, Fluka, 98%). The whole is left for 24 h at 110° C. in a 23-ml Teflon container that is put in a PAAR metal bomb. The solid is then filtered and washed with acetone.

The solid is heated at 120° C. under vacuum for 16 h to remove the acid remaining in the pores.

Lattice parameters of the solid MIL-101(Fe) at 298 K: a=89.0 Å and V=707000 A³, space group Fd-3m (No. 227).

The size of the monodispersed particles (PDI=0.086) measured by light scattering is 391 nm.

i) MIL-101-2CF₃ (Fe) or Fe₃O [(CF₃)₂—C₆H₂—(CO₂)₂]₃.X.nH₂O (X=F, Cl, OH)

135 mg (0.5 mmol) of FeCl₃.6H₂O and 151 mg of 2,5-diperfluoro-1,4-benzenedicarboxylic acid (0.5 mmol, 2CF₃-1,4-BDC, synthesized according to synthesis B described in example 1) are dispersed in 5 ml of DMF (Fluka, 98%). The whole is left for 12 h at 90° C. in a 23-ml Teflon container that is put in a PAAR metal bomb. The solid is then recovered by centrifugation at 10000 rpm for 10 min.

Lattice parameters of the solid MIL-101(Fe) at 298 K: a=89.0 Å and V=707000 A³, space group Fd-3m (No. 227)

The size of the monodispersed particles (PDI=0.145) measured by light scattering is 340 nm.

### Example 5

### Determination of the Iron Content of the Solid MIL-100(Fe) Usable in the Present Invention

### Sample Preparation:

The samples are pressed in the form of self-supporting disks. The disk has a diameter of 1.6 cm and a mass of 13 to 20 milligrams. The tableting pressure is of the order of 10⁹ Pa.

### Activation:

In order to empty the pores of the material (solvents, residual acids) and release the metal coordination sites, the material MIL-100(Fe) was activated by heating at 150° C. under secondary vacuum, i.e. at 10⁻⁶ Pa, for 3 hours. The resultant solid only has iron with a degree of oxidation+III.

Fe³⁺/Fe²⁺ reduction:

Partial reduction of the material MIL-100(Fe) was effected by heating at 250° C. under residual vacuum (about 10⁻³ Pa, i.e. about 10⁻⁵ torr) for 12 hours. Infrared spectroscopy was used for quantifying the relative content of coordinately unsaturated iron(II) sites/coordinately unsaturated iron(III) sites around 20/80% (FIG. 55).

FIG. 55 shows the quantity of coordinately unsaturated iron sites present in the activated solid MIL-100(Fe) as a function of the thermal treatment carried out. The solid MIL-100(Fe) is activated under residual vacuum, i.e. about 10⁻³ Pa, or about 10⁻⁵ torr, at different temperatures and for different times. T(Fe) represents the content of coordinately unsaturated iron sites and T(Fe²⁺) represents the content of coordinately unsaturated Fe²⁺ sites (in μmol of unsaturated sites per gram of activated solid or as percentage of total iron sites).

The amounts of unsaturated iron sites are determined by CO adsorption at 100K followed by infrared spectroscopy. The uncertainty of the values is estimated at ±10%.

### Equipment Used:

The pelletized sample is put in an infrared cell that was designed at the laboratory. The cell can be metallic for studies under a gas stream. The description of the cell is given in the article T. Lesage et al., Phys. Chem. Chem. Phys. 5 (2003) 4435 [64]. It is made of quartz for studies under vacuum or at gas pressures below atmospheric pressure. The cell, which has an integrated furnace for controlled heating of the samples, is connected to a glass ramp for evacuation and/or introduction of gases on the sample.

The infrared spectra are recorded using a Fourier-transform infrared spectrometer of type Nexus (registered trademark) or Magna-550 (registered trademark) made by the company Thermo Fisher Scientific. The spectrometer is equipped with an infrared detector of the MCT/A type. The infrared spectra are recorded with a resolution of 4 cm⁻¹.

### Gases Used:

The gases used for the infrared experiments are of high purity: carbon monoxide: supplier Alphagaz type N47 of purity >99.997%; nitric oxide: supplier Air Liquide, France purity >99.9%; helium, nitrogen, argon: supplier Air Liquide, purity >99.9%.

All the gases are dried beforehand on a molecular sieve and/or by cryogenic trapping using liquid nitrogen. The nitric oxide is purified by distillation.

### Example 6

### Demonstration of the Flexibility of MOF Solids Usable in the Present Invention

The category of flexible hybrid solids based on trimers of trivalent transition metals (Fe, Cr, V; etc.) is designated MIL-88.

These compounds are typically constructed from trimers of iron octahedra, i.e. three iron atoms connected by a central oxygen and by six carboxylate functions connecting the iron atoms two at a time; a terminal water molecule, coordinated with each iron atom, will then complete the octahedral coordination of the metal.

These trimers are then joined together by aliphatic or aromatic dicarboxylic acids to form the solids MIL-88A, B, C, D and MIL-89 (—A for fumaric acid, —B for terephthalic acid, —C for 2,6-naphthalenedicarboxylic acid, —D for 4,4′-biphenyldicarboxylic acid and MIL-89 for trans, trans-muconic acid), as described in the work by Serre et al., Angew. Chem. Int. Ed. 2004, 43, 6286 [67]. Other analogs with other dicarboxylic acids have also been synthesized and are called MIL-88E, F, G etc.

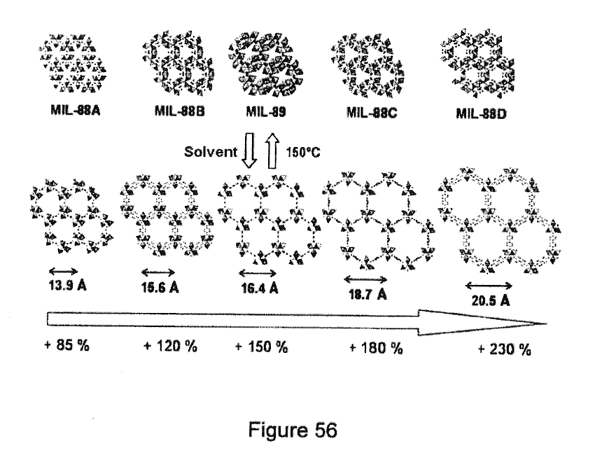

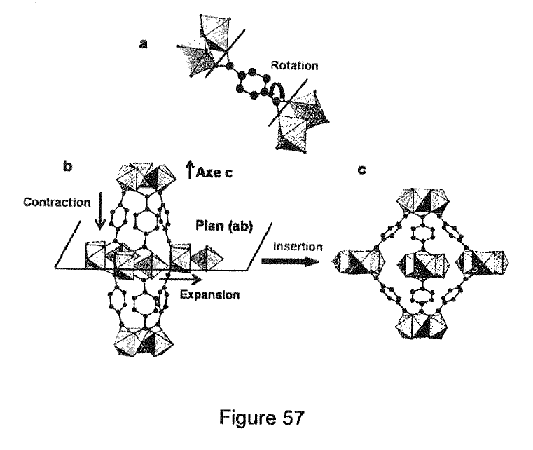

Investigation of the behavior of these solids by X-ray diffraction established that these compounds are flexible with considerable amplitudes of “respiration” (i.e. of swelling or of contraction) between their dry forms and their solvated forms. As a result there are variations in unit cell volume between 85 and 230% depending on the nature of the organic spacer (FIG. 56), as described in the work by Serre et al., Science, 2007, 315, 1828 [91]. The inventors noted that the dry forms are not porous with a roughly identical size of pores (tunnels) regardless of the carboxylic ligand used. In contrast, the swelling of the hybrid solid in liquid phase is a function of the length of the organic spacer. Thus, the distance between trimers in the swollen form ranges from 13.8 Å with fumaric acid (MIL-88A) to 20.5 Å with the biphenyl ligand (MIL-88D). The pore size in the swollen forms thus varies between 7 Å (MIL-88A) and 16 Å (MIL-88D). The swelling is reversible, as shown by the example of the solid MIL-88A in the presence of water in FIG. 57 and also depends on the nature of the solvent used, as described in the work by Serre et al. J. Am. Chem. Soc, 2005, 127, 16273-16278 [92]. The “respiration” takes place continuously, without any apparent bond rupture during respiration. Moreover, on return to room temperature, the solid swells again by resolvation, confirming the reversible character of the respiration.

Close examination of the arrangement of the trimers making up the structure shows that each trimer is connected to six other trimers, three below and three above, by the dicarboxylates, which leads to the formation of bipyramidal cages of trimers. Within these cages, connection between the trimers only takes place along axis c and the absence of any bond in the plane (ab) accounts for the flexibility (FIG. 57).

TABLE 10

| | | a | c | V | Expansion | Estimated | |
|---|---|---|---|---|---|---|---|
| Solid | Condition | (Å) | (Å) | (Å³) | of the cell | pore size | Solvent |
| MIL- | 100° C. | 9.6 | 14.8 | 1180 | >80% | about | Water |
| 88A | 25° C. | 11.1 | 14.5 | 1480 | | 6 Å | |
| | Open form | 13.8 | 12.5 | 2100 | | | |
| MIL- | 100° C. | 9.6 | 19.1 | 1500 | >100% | about | Ethanol |
| 88B | 25° C. | 11.0 | 19.0 | 2000 | | 9 Å | |
| | Open form | 15.7 | 14.0 | 3100 | | | |
| MIL- | 100° C. | 9.9 | 23.8 | 2020 | >170% | about | Pyridine |
| 88C | 25° C. | 10.2 | 23.6 | 2100 | | 13 Å | |
| | Open form | 18.7 | 18.8 | 5600 | | | |
| MIL- | 100° C. | 10.1 | 27.8 | 2480 | >220% | about | Ethanol |
| 88D | 25° C. | 12.1 | 27.5 | 3500 | | 16 Å | |
| | Open form | 20.5 | 22.4 | 8100 | | | |

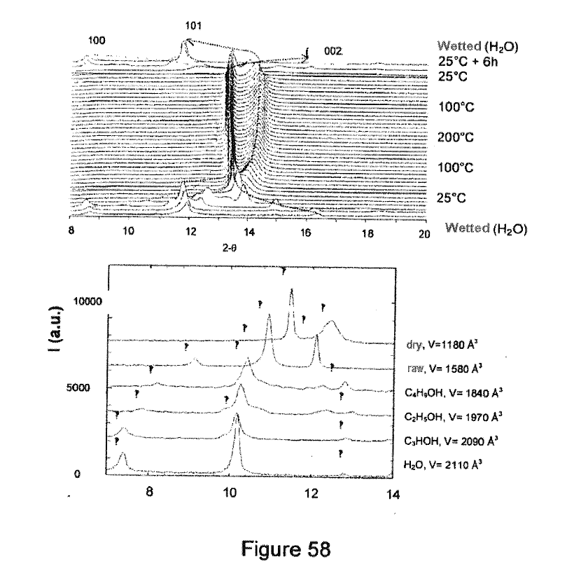

In fact, when a solvent is inserted into the material, the cage deforms with the distance between the trimers decreasing along axis c and increasing in directions a and b, which causes an overall increase in volume of the cage (FIG. 58). Finally, the flexibility of these hybrid solids is remarkable, but is comparable to that of certain polymers. The main difference relates to the crystallinity of the hybrid solids, the polymers being amorphous. Finally, in contrast to the polymers, swelling in the hybrid solids is anisotropic.

TABLE 11
“MIL” structures of some iron(III)
carboxylates according to the invention

| Nanosolid | | |
|---|---|---|
| MIL-n | Organic fraction | Formula |
| MIL-88A | Fumaric acid | Fe₃OX[O₂C—C₂H₂—CO₂]₃•nH₂O |
| MIL-88B | Terephthalic acid | Fe₃OX[O₂C—C₆H₄—CO₂]₃•nH₂O |
| MIL-89 | Muconic acid | Fe₃OCl[O₂C—C₄H₄—CO₂]₃•nH₂O |
| MIL-100 | 1,3,5-Benzene | Fe₃OX[C₆H₃—[CO₂]₃]•nH₂O |
| | tricarboxylic acid | |
| | (1,4-BTC acid) | |
| MIL-101 | Terephthalic acid | Fe₃OX[O₂C—C₆H₄—CO₂]₃•nH₂O |

TABLE 12
Characteristics of the “MIL” structures of iron(III) carboxylates

| | | Pore | | |
|---|---|---|---|---|
| | | diameter | | |
| MIL-n | Iron, %* | (Å)** | Flexibility | Metal base |
| MIL-88A | 30.8% | 6 | yes | Octahedral |
| | | | | trimer |
| MIL-88B | 24.2% | 9 | yes | Octahedral |
| | | | | trimer |
| MIL-89 | 26.2% | 11 | yes | Octahedral |
| | | | | trimer |
| MIL-100 | 27.3% | 25-29 | no | Octahedral |
| | | | | trimer |
| MIL-101 | 24.2% | 29-34 | no | Octahedral |
| | | | | trimer |

*theoretical percentage of iron in the dry phase
**pore size calculated from the crystallographic structures

### Example 7

### Reduction of Nitrogen Oxide According to the Invention with an MOF Based on Iron Carboxylate (MIL-100 (Fe)) and Temperature Tests

Tests for reduction of nitrogen oxide were carried out on a sample of fluorinated MIL-100 (Fe) described in example 1 formed into a self-supporting tablet obtained by pressing the powder of the sample between two steel mirrors placed in a cylinder with a piston, connected to a hydraulic press. The disk has a diameter of 1.3 cm and a mass varying from 13 to 20 milligrams during the experiments. The tableting pressure is of the order of 10⁹ Pa.

As in example 5, the pelletized sample is put in an infrared reaction cell designed at the laboratory or in a commercial reaction cell made by Aabspec, model #CX positioned in an FT-IR spectrometer and connected to a system for introduction and analysis of gas phases. The cell is connected to a system of metal pipes for studies under a gas stream (synthesis gas bench). The system used is described in the article T. Lesage et al., Phys. Chem. Chem. Phys. 5 (2003) 4435 [64]. The concentrations of the gases are obtained by means of mass flowmeters of the Brooks type, operated electronically. The gaseous effluents are analyzed by a gas infrared microcell (with the trade name Nicolet, Thermo Scientific) and by a Pfeiffer Omnistar quadrupole mass spectrometer connected in line.

The infrared spectra of the specimen surface and of the gas phase are recorded with a Nexus (registered trademark) Fourier-transform infrared spectrometer manufactured by the company Nicolet, Thermo Scientific. The spectrometer is equipped with an infrared detector of the MCT/A type. The infrared spectra are recorded with a resolution of 4 cm⁻¹ after accumulation of 64 scans.

The gases used for the infrared experiments are of high purity: carbon monoxide: supplier Alphagaz type N47 purity (>99.997%); nitric oxide: supplier Air Liquide, France purity >99.9%; helium, nitrogen, argon: supplier Air Liquide, purity >99.9%. All the gases are dried beforehand on a molecular sieve. The water is introduced into the gas mixture in a controlled manner, via a thermostatically-controlled saturator, where distilled water vapor is entrained by a carrier gas (Ar).

The cell containing the sample was first purged by a stream of dry argon at 25 mL/min for 3 h at 250° C., then cooled to room temperature, still under the argon stream. A mixture of 500 ppm of NO and 1% of water plus argon as carrier gas, making up a total of 25 mL/min, in order to obtain a liquid hourly space velocity (LHSV) of 100-150000 h⁻¹, representative of an engine exhaust, was sent onto the sample at 25° C., 100° C., 150° C., 200° C. and 250° C.

At each temperature plateau, after an initial phase of absorption of NO in the form of nitrosyls on Fe²⁺ cations, the gas stream stabilized at a steady-state composition (analyzed by IR and by mass spectrometry).

The experiment shows stable, catalytic conversion of NO to N₂ of 2.8% at 25° C. and of 1% at 100° C., as well as of NO to N₂O predominantly, from 1.5 to 2%, at 250° C.

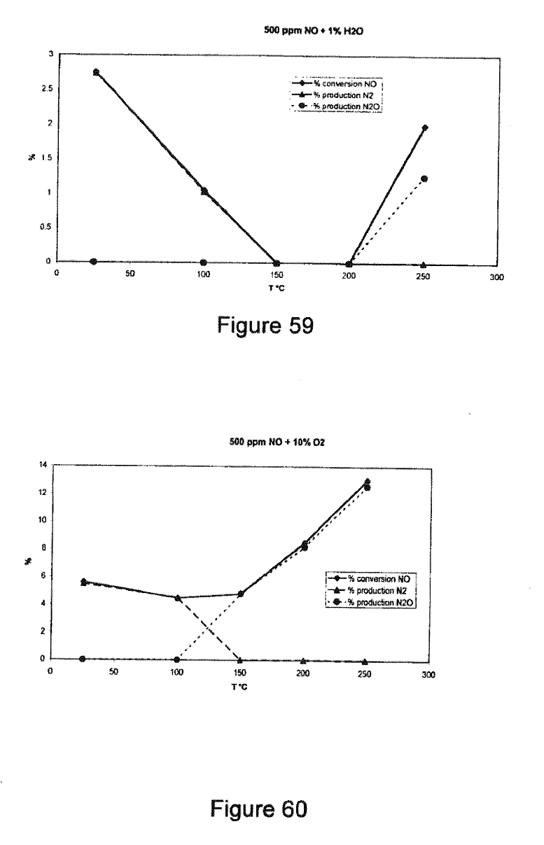

FIG. 59 is a graph showing the experimental results of the variation of the NOx sent onto the sample and of their conversion to N₂ and N₂O as a function of the temperature.

TABLE 13
Evolution of NOx stored on the sample and conversion
to N₂ and N₂O as a function of temperature under a
flow of 500 ppm of NO and 1% of water in Ar

| | | | | % N₂ |
|---|---|---|---|---|
| Temper- | Amount of NOx | % NO absent | % N₂O | (NO |
| ature | stored/g of | in outlet | (NO | equivalent) |
| (° C.) | catalyst | stream | equivalent) | mass 28 |
| 25 | 69.80 | 2.75 | 0.00 | 2.74 |
| 100 | 59.99 | 1.05 | 0.00 | 1.03 |
| 150 | 31.14 | 0.00 | 0.00 | 0 |
| 200 | 24.06 | 0.00 | 0.00 | 0 |
| 250 | 21.02 | 1.99 | 1.25 | 0 |

Alternatively, a mixture of 500 ppm of NO and 10% of dioxygen plus Ar as carrier gas, to a total of 25 mL/min, was sent at 25° C., 100° C., 150° C., 200° C. and 250° C., in the same conditions as those specified above, onto a sample that had been pretreated as in the preceding case.

At each temperature plateau, after an initial phase of NO absorption in the form of nitrosyls, the gas stream stabilized to a steady-state composition analyzed by IR and by mass spectrometry. The experiment shows stable conversion of NO to N₂ of 5.6% at 25° C. and of 4.5% at 100° C., as well as of NO to N₂O of 4.8% at 150° C., of 8.5% at 200° C. and of 13% at 250° C.

TABLE 14
Evolution of NOx stored on the sample and conversion
to N₂ and N₂O as a function of temperature under a flow
of 500 ppm of NO and 10% of O₂ in Ar

| | | | | % N₂ |
|---|---|---|---|---|
| Temper- | Amount of NOx | % NO absent | % N₂O | (NO |
| ature | stored/g of | in outlet | (NO | equivalent) |
| (° C.) | catalyst | stream | equivalent) | mass 28 |
| 25 | 97.70 | 5.62 | 0.00 | 5.5 |
| 100 | 18.89 | 4.47 | 0.00 | 4.5 |
| 150 | 11.97 | 4.79 | 4.82 | 0 |
| 200 | 9.07 | 8.50 | 8.19 | 0 |
| 250 | 13.91 | 13.02 | 12.59 | 0 |

It can be seen that, in the conditions used at present, there is greater conversion of NO to N₂ at low temperatures.

At higher temperatures, reduction of NO is partial and gives rise to N₂O, with conversions of about 5%, 8.5% and 13% at 150° C., 200° C. and 250° C. respectively.

FIG. 60 is a graph showing the experimental results of the evolution of the NOx sent onto the sample and of their conversion to N₂ and N₂O as a function of temperature.

At the same time, on the surface, we mainly observe the presence of mononitrosyls on Fe²⁺, as well as traces of mononitrosyls on Fe³⁺ and/or of dinitrosyls.

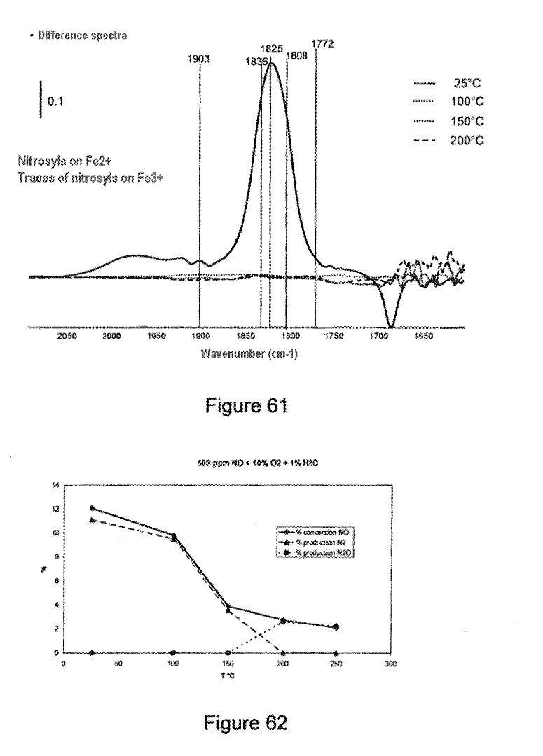

FIG. 61 shows the differential IR spectra of the species adsorbed on the surface of the sample under a reaction flow of 500 ppm of NO and 10% of O₂ in Ar.

### Example 8

### Reduction of Nitrogen Oxide According to the Invention

In the following example the stream contains 500 ppm NO, 10% O₂ and 1% H₂O in Ar and it is passed through the MOF used in example 7 from room temperature to 250° C., in stages of 50° C., in the same experimental conditions as for example 7.

Apart from small amounts of nitrous oxide at 200 and 250° C., no conversion of NO is observed; the amount of NO stored on the surface of the sample is also small and is probably in the form of N₂O₄.

When the sample is cooled, selective catalytic reduction appears, having values of about 4% at 150° C., 10% at 100° C. and 12% at 25° C.

These values are significant and substantial, greater than those obtained by photocatalysis on TiO₂, where the major part of the method is in fact based on adsorption of NOx in the form of nitrates on the titanium dioxide and its subsequent removal by decomposition at high temperature or washing with formation of nitric acid, as explained for example in B. N. Shelimov et al., Journal of Photochemistry and Photobiology A-Chemistry, 195 (2008) 81 [93].

The experimental results of this example are shown in the accompanying FIG. 62 and in Table 15, given below.

FIG. 62 shows the activity of the sample MIL-100 (Fe) in reduction of the NOx as a function of temperature, on plateaux at 250° C., 200° C., 150° C., 100° C. and 25° C. At each temperature plateau, after an initial phase of absorption of NO in the form of nitrosyls, the gas stream stabilized to a steady-state composition (analyzed by IR and by mass spectrometry). The experiment shows stable conversion of NO to N₂O of about 2.2% at 250° C. and of 2.6% at 200° C., as well as of NO to N₂ of 3.6% at 150° C., of 9.5% at 100° C. and of 11.1% at 25° C., always in the absence of reducing agent.

TABLE 15
Evolution of NOx stored on the sample and conversion
to N₂ or N₂O as a function of temperature, under a stream of
500 ppm of NO, 1% of water and 10% of O₂ in Ar

| | | | | % N₂O |
|---|---|---|---|---|
| Temper- | Amount of NOx | % NO absent | % N₂O | (NO |
| ature | stored/g of | in outlet | (NO | equivalent) |
| (° C.) | catalyst | stream | equivalent) | mass 28 |
| 25 | 16.93 | 0.00 | / | / |
| 100 | 11.96 | 0.00 | / | / |
| 150 | 7.84 | 0.00 | / | / |
| 200 | 3.64 | 0.00 | / | / |
| 250 | 5.76 | 2.10 | 2.22 | 0 |
| 200 | 7.61 | 2.73 | 2.6 | 0.00 |
| 150 | 14.36 | 3.89 | 0 | 3.55 |
| 100 | 19.36 | 9.81 | 0 | 9.50 |
| 25 | 18.92 | 12.07 | 0 | 11.10 |

After verification, the inventors confirm that the structure of the material is intact after all treatment under the reaction stream, as described above.

The inventors note once again that reduction of NO is obtained in the absence of gaseous reducing agents, at low temperature, in the presence of Fe²⁺ and in particular of Fe²⁺/Fe³⁺ pairs.

NO has a reducing power on the iron ions in this structure [93], which creates redox pairs capable of effecting and maintaining the dissociation

2NO→N₂+O₂

The presence of an oxidant such as oxygen and/or water in the reaction stream does not have a negative effect on dissociation.

### Example 9

### Change in Space Velocity and Composition of the Gases

Other experiments were carried out at different space velocities, with different contents of nitric oxide, water and oxygen on the MOF material tested in example 8.

On decreasing the LHSV between 5000 and 20000 h⁻¹, this material attains far superior performance, even at very high concentrations of NO (900 ppm).

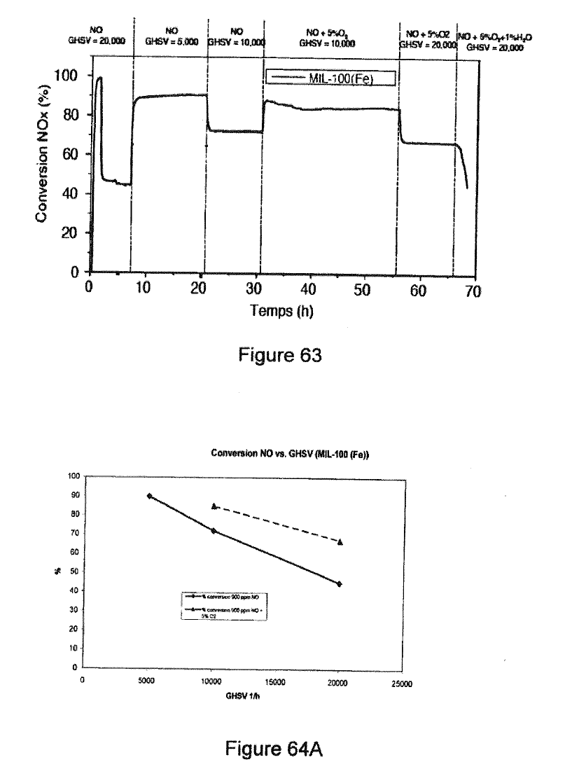

FIG. 63 is a graph showing the experimental results of conversion of 900 ppm of NO at 30° C., after pretreatment of the sample at 250° C. for 6 hours, at space velocities between 5000 and 20000 h⁻¹. The same experiments are performed in the presence of oxygen and water.

In this example, a sample of MIL-100 (Fe) of about 1.5 g was put in a tubular reactor connected to a system for introduction and analysis of gas phases (by gas chromatography and mass spectrometry). The sample was pretreated by passing a helium stream (100 mL/min) over it for 6 h at 250° C. The experiment was then conducted at 30° C., under a mixture of 900 ppm of NO and He as carrier gas, to a total of 100 mL/min.

After an initial absorption phase, the experiment showed stable conversion of NO for at least 10 to 20 hours of 90% at a space velocity of 5000 h⁻¹, of 72% at 10000 h⁻¹ and of 45% at 20000 h⁻¹.

On introduction of a mixture of 900 ppm of NO, 5% O₂ and He as carrier gas, to a total of 100 mL/min, conversion increased to 85% and 67% for a space velocity of 10000 h⁻¹ and 20000 h⁻¹, respectively.

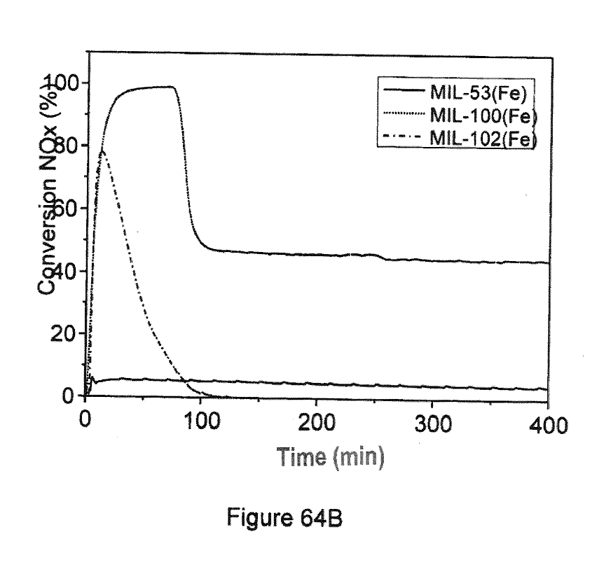

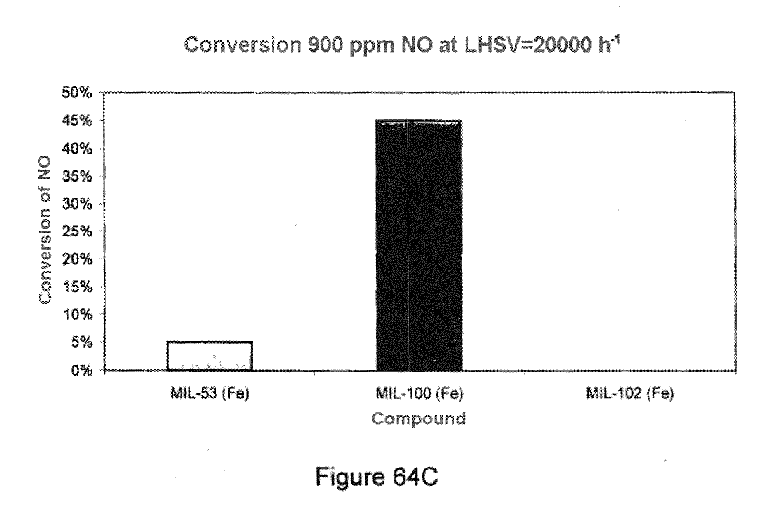

As shown in FIG. 64A, conversion of NO varies between 45% and 90% depending on the space velocity. The addition of 5% of oxygen improves the activity (as already noted in the previous experiments) at constant space velocity. The subsequent addition of 1% of water lowers the conversion considerably.

Similar tests were carried out on the other two samples: MIL-53 (Fe) obtained according to the protocol described in T. R. Whitfield et al., Metal-organic frameworks based on iron oxide octahedral chains connected by benzenedicarboxylate dianions, Solid State Sci., 7, 1096-1103, 2005 [94]; and MIL-102 (Fe) obtained according to the protocol described in S. Surblé et al., J. Am. Chem. Soc. 128 (2006), 46, 14889 [72].

FIG. 64B is a graph showing the experimental results for conversion of 900 ppm of NO at 30° C., on different materials after sample pretreatment at 250° C. for 6 hours, at a space velocity of 20000 h⁻¹. These results are also presented in FIG. 64C in the form of a histogram.

It can be seen that MIL-53 displays very limited activity (˜5%), whereas MIL-102 (Fe) only shows capacity for adsorption of NO and catalytic reduction activity of less than 5%, while MIL-100 is able to dissociate more than 45% of NO (FIG. 64B and FIG. 64C).

These results can certainly be linked to different accessibility of the iron polyhedra in the hybrid structures.

### Example 10

### Relation Between Catalytic Activity and Space Velocity for Reduction of Nitrogen Oxides According to the Invention

In this example, a sample of MIL-100 (Fe) manufactured as above of about 1.5 g was put in a tubular reactor connected to a system for introduction and analysis of gas phases by gas chromatography and mass spectrometry.

The sample was pretreated by passing a helium stream of 100 mL/min over it for 6 h at 250° C.

The experiment was then conducted at 30° C., under a mixture of 900 ppm of NO and He as carrier gas, to a total of 100 mL/min.

After an initial absorption phase, the experiment showed stable conversion of NO for at least 20 h of 90% at a space velocity of 5000 h⁻¹, of 72% at 10000 h⁻¹ and of 45% at 20000 h⁻¹.

On introduction of a mixture of 900 ppm of NO, 5% O₂ and He as carrier gas, to a total of 100 mL/min, conversion increased to 85% and 67% for a space velocity of 10000 h⁻¹ and of 20000 h⁻¹, respectively.

The results of these experiments are shown in FIG. 64A which presents the percentage conversion of NO (disappearance of NO) as a function of the space velocity in h⁻¹.

### Example 11

### Application of the Present Invention with Different Concentrations of Nitrogen Oxide

This example presents another method of synthesis of porous iron trimesate MIL-100 and its catalytic activity for conversion of NOx.

The fluorinated solid MIL-100(Fe) or F-MIL-100(Fe) is obtained by hydrothermal reaction of trimesic acid with metallic iron, HF, nitric acid and water. The proportions of the reaction mixture are: 1.0 Fe°: 0.67 1,3,5-BTC: 2.0 HF: 0.6 HNO₃: 277 H₂O (1,3,5-BTC=1,3,5-benzenetricarboxylic acid or trimesic acid). The mixture of the reactants is maintained at 150° C. in a Teflon-lined autoclave for 12 hours. The pH remains acid throughout the synthesis.

A clear orange product is recovered by filtration and is washed with deionized water. Then the F-MIL-100(Fe) is purified by a two-step method using water at 80° C. for 5 h and ethanol at 60° C. for 3 h, thus obtaining a very pure compound MIL-100(Fe). The sample is finally dried overnight at less than 100° C. under a nitrogen atmosphere.

In the same way as in example 10, the solid F-MIL-100(Fe) obtained was used for catalytic conversion of NOx, except for a different concentration of NO₂.

In this example, 2000 ppm and 500 ppm of NO₂ were used during the reaction.

The degree of conversion of the NO₂ converted (removed) is about 95% from an initial level of 2000 ppm of NO₂ whereas the conversion is 99% from an initial dose of 500 ppm.

### Example 12

### Application of the Present Invention at Different Temperatures

In the same experimental conditions as in example 10, the solid F-MIL-100(Fe) is used for catalytic conversion of NOx, varying the parameter temperature.

In this example, the temperature of the catalytic reaction is set at different temperatures selected between 50 and 150° C.

The degree of conversion of NO₂ is for example 70% at 70° C. and 60% at 100° C.

### Example 13

### Application of the Present Invention with Different Flow Rates of Nitrogen Oxide

In the same experimental conditions as in example 10, the solid F-MIL-100(Fe) is used for catalytic conversion of NOx, varying the parameter flow rate of nitrogen oxide.

In this example, the total flow rate of reactive gas mixture varies from 150 to 300 ml/min.

The degree of conversion of NO₂ is 95% for a flow rate of 150 ml/min, i.e. 1000 ppm NO₂, and 85% for a flow of 300 ml/min with 1000 ppm of NO₂.

### Example 14

### Application of the Present Invention with a Mixture of Nitrogen Oxides

In the same experimental conditions as in example 11, the solid F-MIL-100(Fe) is used for catalytic conversion of NOx, this time with an initial mixture of NO and NO₂.

In this example, the mixture introduced contains 810 ppm of NO, 240 ppm NO₂, with 5 vol. % of O₂, and 1 vol. % of H₂O, the whole carried by helium.

1.5 g of catalyst is used, with a total gas flow of 100 ml/min.

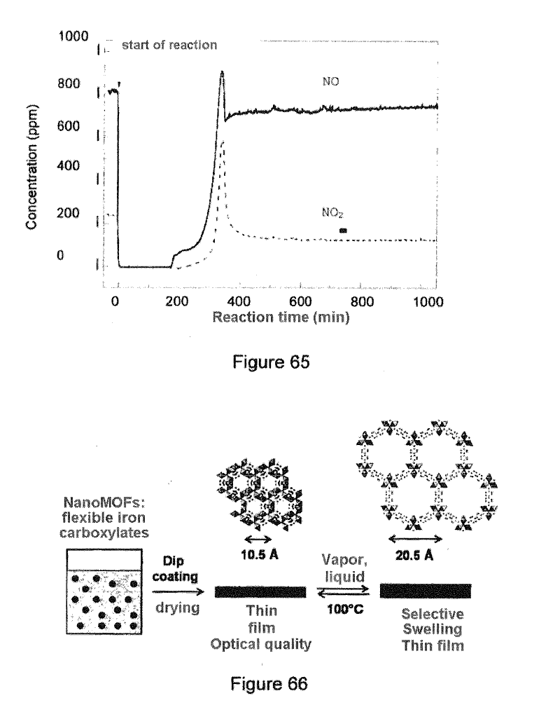

As shown in FIG. 65, NO and NO₂ disappeared completely at reactor outlet once the reaction mixture was introduced into the reactor, thus indicating their adsorption within the pores of the material. The adsorption step is extended to a duration of 170 min in conditions of continuous flow. After saturation of adsorption, large amounts of NO and NO₂ are released suddenly, giving a maximum of 900 ppm of NO and 590 ppm of NO₂ at reactor outlet for a duration of up to 340 min under flux. Then the concentration levels stabilize after 450 min under flux. At equilibrium, the concentrations of NO and NO₂ are 720 ppm of NO, 125 ppm of NO₂, respectively, corresponding to a degree of conversion of 11% (NO) and 48% (NO₂). The catalyst displays stable activity for about 1000 min. At the same time, the production of N₂ is also visualized by gas chromatography.

### Example 15

### Application of the Present Invention in the Presence of Oxygen

In this example, the solid F-MIL-100(Fe) is used for conversion of NO in the presence of oxygen.

The catalyst is activated beforehand in a helium stream of 100 ml/min at 250° C. for 3 hours. The mixture contains 810 ppm of NO and 10 vol. % of O₂, the whole carried by helium.

The weight of the catalyst used is 0.6 g and the total gas flow is 100 ml/min.

The initial mixture is introduced into a reactor containing the catalyst and the gas NO disappears completely at reactor outlet for a period of time of the order of 480 min.

After saturation by adsorption, the concentration of NO rises again to 145 ppm and most of the NO is converted to NO₂.

This result indicates in particular that the solid F-MIL-100(Fe) is active for oxidation of NO to NO₂ in the presence of oxygen. This activity as oxidant is associated with the activation treatment at 250° C.

### Examples 16

### Catalysis with MOFs Other than Those in the Preceding Examples

### Example 16 (A)

This example presents another method of synthesis of porous iron trimesate MIL-100 and its catalytic activity for conversion of NOx.

The nonfluorinated solid MIL-100(Fe) or N-MIL-100(Fe) is obtained in the form of polycrystalline powder from the following initial reaction mixture: 1.0 FeNO₃.9H₂O: 0.66 1,3,5-BTC: 54.5 H₂O (1,3,5-BTC: 1,3,5-benzenetricarboxylic acid or trimesic acid), the whole being maintained at 160° C. in a Teflon-lined autoclave for 12 hours with an initial heating ramp of 6 hours and a final cooling ramp of 12 hours.

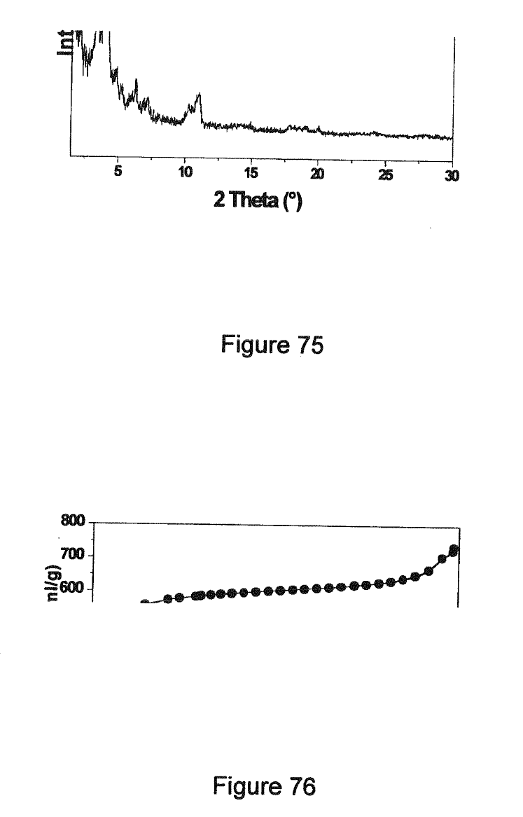

The results of this experiment are shown in FIG. 75 (DRX). This figure shows an X-ray diffraction pattern of the nonfluorinated solid MIL-100(Fe) or N-MIL-100(Fe).

An orange solid is recovered by filtration and washed with deionized water. It is treated in a mixture of deionized water and ethanol for 3 hours at 80° C. so as to lower the residual amount of trimesic acid in the pores of the MOF, followed by drying at room temperature.

After this treatment, there is still free acid and an additional purification step is carried out. The solid is dispersed in a solution 1 mol/l of aqueous solution of NH₄F at 70° C. for 24 hours and immediately filtered hot and washed with hot water. The solid is finally dried overnight at 100° C. in a stove.

The catalyst N-MIL-100(Fe) (see the appended FIG. 76) has a BET specific surface of 1970 m²/g with a pore volume of 1.13 ml/g. FIG. 76 shows a nitrogen N₂ adsorption isotherm of the nonfluorinated solid MIL-100(Fe) or N-MIL-100(Fe). (P₀=1 atmosphere)

N-MIL-100(Fe) is then used for catalytic conversion of a mixture of NOx (NO and NO₂).

In this example, the initial mixture contains 690 ppm of NO and 320 ppm of NO₂, 5 vol. % of O₂, and 1 vol. % of H₂O, the whole carried by helium. The weight of catalyst used is 1.5 g and the total gas flow is 100 ml/min.

At equilibrium, the concentrations of NO and NO₂ are 650 ppm for NO and 150 ppm for NO₂, respectively, corresponding to a conversion of 6% for NO and 53% for NO₂.

### Example 16(B)

### MOF

In this example, another MOF containing a cation other than iron was also tested in the same reaction conditions.

The solid HKUST-1 (Cu₃[(CO₂)₃C₆H₃]₂ (H₂O)₃) whose synthesis is described, for example, in S. S.-Y. Chui et al., Science, 283, 1148-1150: A chemically functionalizable material [Cu₃(TMA)₂(H₂O)₃]_{n [}96] was pretreated at 250° C. in a stream of NO (900 ppm) for 6 h, then submitted to the reaction mixture as in example 9. It shows a conversion of NO of 33% at 30° C., for a space velocity of 10000 h⁻¹.

### Example 17

### Activation of an MOF Solid for Application of the Present Invention

The solid, already formed, is activated either by pumping under secondary vacuum at about 10⁻³ Pa (i.e. about 10⁻⁵ torr) at 250° C. for 3 hours, or under a stream of dry inert gas or of NO, at 250° C. for 6 hours.

This activation makes it possible to remove some or all of the residual impurities originating from the process for manufacture of the MOF or storage thereof. These impurities can be acid, water, etc.

This activation also makes it possible to transform some of the Fe³⁺ sites in the MOF to Fe²⁺ sites.

It can clearly be seen that modifying the structure has an influence on the properties of decomposition, especially in the presence of water and/or other gases, such as CO₂, CO, SO₂ and unburnt hydrocarbons.

In conclusion, the inventors have demonstrated that appropriate activation of an MOF solid makes it possible, quite remarkably, to catalyze the room-temperature transformation of NOx, in the presence of oxygen and water, without the need to add a reducing agent.

### Example 18

### Coating of a Surface with an MOF Solid for a Use According to the Present Invention

A thin layer of the flexible solid MIL-89, a porous iron muconate, was prepared from a colloidal solution of iron(III) acetate (obtained by the method described in Dziobkowski et al. [87]) and of muconic acid in ethanol.

Preparation uses 172 mg of iron(III) acetate, 85 mg of trans, trans-muconic acid (Fluka, 97%) dissolved with stirring at room temperature in 15 mL of absolute ethanol (Aldrich, 99%). The solution is then heated at 60° C. for 10 minutes in static conditions, quickly leading to an increase in viscosity and turbidity of the solution, which accords with the presence of colloidal nanoparticles.

The thin layer of the MOF solid is then prepared by dip-coating in the colloidal solution previously heated for 10 minutes at 60° C., using a polished silicon substrate and a shrinkage rate of 4 mm.s⁻¹ at relative humidity of 15% (FIG. 66).

The film is then maintained for a further 2 minutes at this humidity before being washed with ethanol and dried at room temperature or for 5 minutes at 130° C. in air, which does not affect the final structure.

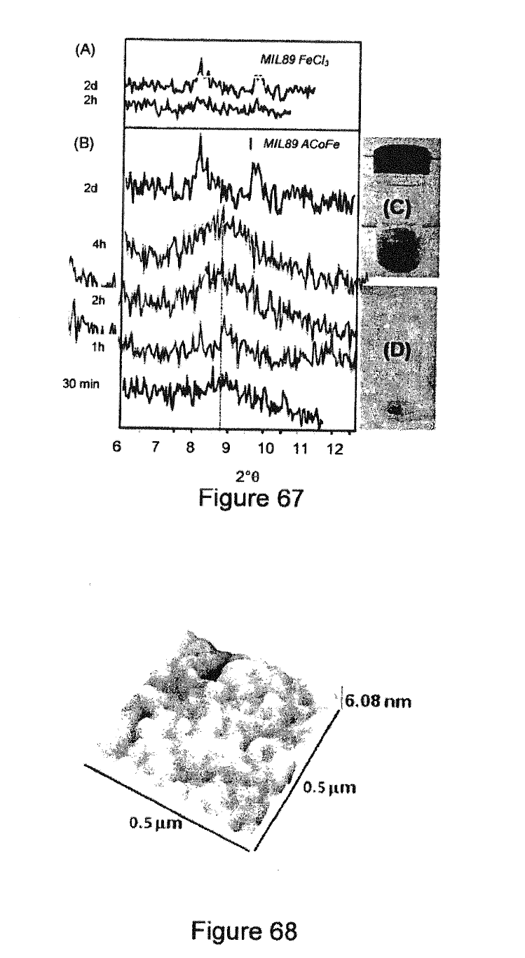

These results are shown in the appended FIG. 67. This figure shows, on the left, the X-ray diffraction patterns (λ_{Cu}=1.5406 Å) of the nanoparticles of the solid MIL-89 obtained from the gel used for making the thin layers described above:
(A) particles obtained from precursor FeCl₃;
(B) particles obtained from iron acetate;
(C) photograph of the gel of MIL-89 obtained after 10 minutes at 60° C. and 2 days at room temperature (25° C.);
(D) monolith of MIL-89 formed after 10 minutes at 60° C. and 3 months at room temperature (25° C.)

The thickness of the layer obtained in this example is about 40 nm for a monolayer in the conditions mentioned above.

The appended FIG. 68 is a micrograph of atomic force electron microscopy (AFM) of a thin layer of the solid MIL-89 obtained by the method described above. The AFM micrographs were obtained using a microscope Veeco DI CPII (brand name) with a tip of silicone MPP-11120, in noncompact mode and with an acquisition rate 1r 1 μm/1 μm.

In fact, as a function of time, we observe maturation of the starting gel, leading to the formation of larger particles.

Larger thicknesses are obtained by repeated depositions, for example using the same conditions.

This example shows the possibility of forming a coating of an MOF solid on a surface for application of the present invention.

### Example 19

### Example of Recycling of an MOF Solid Usable in the Present Invention

The components of the porous hybrid solid (MOF) in the above examples are recovered after catalysis for nitrogen oxide removal.

The following method is used:

Various tests with suspension of 1 g of MOF in 50 mL of aqueous solution of hydrochloric acid of concentration 1, 2, 3, 4 and 5 mol/L.

Various heating tests are carried out at 50° C. with stirring for 1 h00 to 12 h00.

The metal of the MOF goes into solution as the ion Mⁿ⁺ (H₂O)x and the carboxylic acid (e.g. trimesic acid), generally very poorly soluble in water in acid conditions, is precipitated.

For example, tests on MIL-100(Fe) showed these results.

Filtration makes it possible to recover the recrystallized acid.

The metal in the form of chloride, for example of iron when M is iron, is then concentrated by evaporation in water in a rotary evaporator and then dried under primary vacuum at 50° C. for 15 hours.

In this way, the starting products are recovered and can be used for synthesizing the MOF again.

### Example 20

### Example of Regeneration of an MOF Solid Usable in the Present Invention

The porous hybrid solids (MOF) described above in the examples are used as deNOx agent, i.e. for catalysis of nitrogen oxide removal.

In this example, tests are applied for regenerating these MOF solids after use. In fact, the inventors noted that after several cycles of use of the MOF, depending on the application conditions, there could be species such as NO, NO₂, or even nitrates etc., which would poison the active sites of the MOF.

### The Following Protocol 1 is Tested:

Suspension of 1 g of the MOF, notably of MIL-100(Fe), in 100 mL of absolute ethanol,
Heating at 80° C. for 2 hours, and
Hot filtration of the product to recover the regenerated MOF.

The species that poison the active sites of the MOF are removed.

### The Following Protocol 2 is Tested:

Pass a flow of steam carried by an inert gas, N₂, over the MOF solid at a temperature of 80° C. for 2 h00.

The species that poison the active sites of the MOF are removed.

### Example 21

### Example of a First Device of the Present Invention

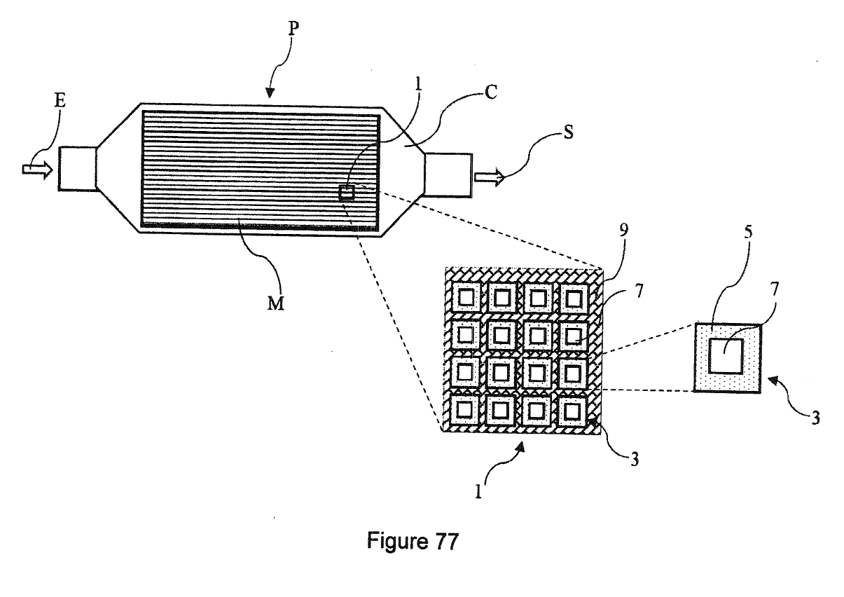

An example of a device (D) according to the present invention (“De-NOx” device) for treating a gaseous or liquid effluent comprising nitrogen oxide to be removed is shown in FIG. 77.

In this figure, the device comprises an MOF solid (5) also called “active phase”. This device also comprises means for contacting (7), (9), (M) said MOF solid with the nitrogen oxide. These means comprise a ceramic honeycomb structure (M) constituted of a base unit (3), longitudinal channels of square shape (7) with walls on which the MOF solid (5) is deposited, said walls being of ceramic (9).

The effluent to be treated passes through the longitudinal channels (7) where it is in contact during said passage with the MOF (5), which catalyzes removal of the nitrogen oxides from the effluent.

This device further comprises an inlet of gaseous or liquid effluent containing nitrogen oxide (E), an outlet (S) of treated effluent no longer containing nitrogen oxide, a stainless steel casing (C) protecting the ceramic monolith (M) supporting the MOF solid.

In FIG. 77, a zone (1) is shown enlarged in cross-section of the monolith (M). This zone 1 clearly shows the honeycomb structure of the monolith constituted of a base unit (3) comprising the contacting means (7) and (9), namely the longitudinal channels (7), the ceramic walls (9) and the MOF (5).

The catalytic converter is constituted here of a monolith (M) of metal or of ceramic, structured as a honeycomb, which contains the active phase, the MOF, deposited on its walls, and protected by a stainless steel casing. The monolith is composed of fine longitudinal channels separated by thin walls. The active phase is deposited on the ceramic support by impregnation by the wash-coating method presented for example in Handbook of Heterogeneous Catalysis, 2^{nd} Edition, G. Ertl, H. Knözinger, F. Schüth, J. Weitkamp Editors, 2008, ISBN: 978-3-527-31241-2 [95].

In another example, the support is made of stainless steel.

The active phase constitutes a thin layer with an average thickness of about 100 μm on the inside walls of the channels.

In another example of a device of the present invention, the channels are of circular shape.

This device can easily be incorporated in a catalytic converter (P) or a duct for leading away a gaseous or liquid effluent from a factory, workshop, laboratory, stored products, urban air vents etc. All that is required is to connect the inlet (E) of this device to a duct conveying the gaseous effluent to be treated. Catalysis for removal is immediate, even at room temperature.

Various devices are made with the various MOFs described in the preceding examples.

### Example 22

### Example of a Second Device of the Present Invention

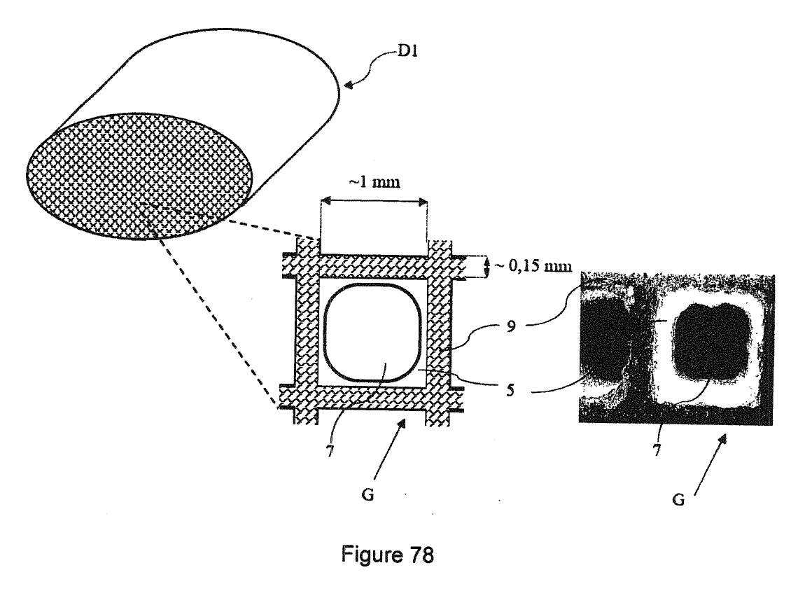

Another example of a device (D1) according to the present invention for treatment of a gaseous or liquid effluent containing nitrogen oxide to be removed is shown in FIG. 78.

In this figure, the device comprises an MOF solid (5). This device also comprises means for contacting (7), (9) said MOF solid with the effluent to be treated. These means comprise a ceramic honeycomb structure (M) comprising longitudinal channels of roughly circular shape (7) on the walls (9) of which the MOF solid (5) is deposited. These walls (9) are of ceramic. In this example, the monolith (M) is a monolith with 60 channels per cm² (i.e. 400 cpsi or 400 channels per square inch).

In FIG. 78, a cross-section (G) of the honeycomb structure is shown schematically and as a photograph. This cross-section clearly shows the base unit constituting the honeycomb, which is square. In this nonlimiting example, this square has a side of 1 mm and its walls have a thickness of about 0.15 mm. The structure of these squares consists, in this nonlimiting example, of a ceramic wall, also called monolithic support (9).

In other devices, the ceramic is replaced with silicon carbide or stainless steel or folded paper or some other suitable support.

The walls are coated with a layer of MOF (5). The MOF is the catalyst for decomposition of nitrogen oxide according to the present invention.

This device can also, like that of example 25, be incorporated very easily in a catalytic converter (P) or a duct conveying a gaseous or liquid effluent from a factory, workshop, laboratory, stored products, urban air vents, or a system for air conditioning of vehicles, of a building etc.

This device notably provides very efficient removal of nitrogen oxides produced by a heat engine or internal combustion engine of a vehicle, by passing the effluent to be treated through the longitudinal channels (7) where it is in contact during said passage with the MOF (5), which catalyzes the removal of nitrogen oxides from the effluent.

This device can also be integrated in an exhaust system of an engine, for example in a muffler.

Various devices are made with the various MOFs described in the preceding examples.

### Example 23

### Example of a Catalytic Converter of the Present Invention Installed in an Exhaust System

Another example of a device according to the present invention is shown in FIG. 80. In this figure, it bears the reference (15) and it is installed in an exhaust system in the form of a catalytic converter.

In this example, an exhaust system is constructed with a device according to the invention described in example 21 and an exhaust system is constructed with a device according to the invention described in example 22.

In this example, the exhaust system shown in this figure further comprises a flange for connection to the exhaust manifold (11), an expansion box (13), a rear muffler (17) and an exhaust muffler (19).

In this example, the device of the invention is arranged in the exhaust system between the expansion box (13) and the rear muffler (17).

Various exhaust systems are constructed, in which the device is situated at another place in the exhaust system, i.e. between the connection to the exhaust manifold (11) and the expansion box (13) and between the rear muffler (17) and the exhaust muffler (19).

The catalytic converter, which performs several functions and can be divided physically into several blocks, is generally located between the manifold at engine outlet and the muffler. The precise position is determined essentially in relation to the exhaust gas temperature that we wish to have within the catalysts.

The nitrogen oxide is removed from a liquid or gaseous effluent simply by passage through the device.

Various devices are made with the various MOFs described in the preceding examples.

### Example 24

### Example of a Catalytic Converter of the Present Invention Incorporated in an Engine

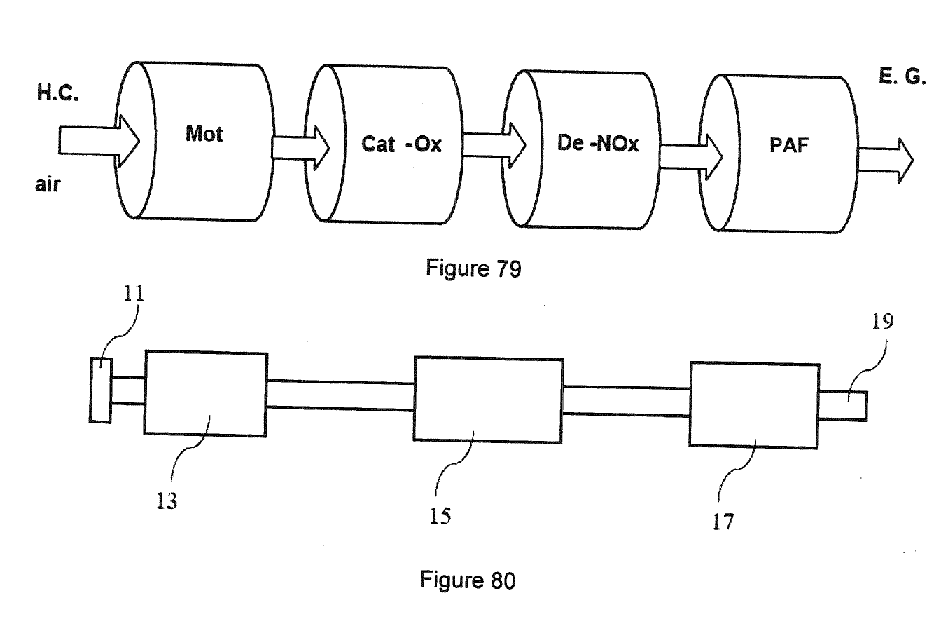

In this example, a catalytic converter (De-NOx) according to the present invention is incorporated in an exhaust system of an engine (Mot). The device obtained is shown in FIG. 79. This device comprises a device (D) or (D1) described in example 21 or 22 comprising an MOF solid and means for contacting said MOF solid with the nitrogen oxide corresponding to those shown in FIG. 77 or 78.

The device according to the invention is situated downstream of an inlet for air and hydrocarbons (H.C), of an engine (Mot) generating exhaust gas comprising nitrogen oxides, and a device for oxidation of carbon dioxide (CO) and for oxidation of the hydrocarbons (Cat-Ox). A particulate filter (PAF) for removing soot and a gas exhaust orifice can be positioned downstream or upstream of the device (De-NOx) according to the invention.

This device makes it possible to remove the nitrogen oxides produced by an engine, by passing the effluent to be treated through the longitudinal channels (7), where it is in contact, during said passage, with the MOF (5), which catalyzes removal of the nitrogen oxides that it contains.

The various functions performed within the catalytic converter therefore include:
1. oxidation of CO emitted by the engine as a result of incomplete combustion of carbon-containing products;
2. oxidation of unburnt hydrocarbons;
3. reduction of the nitrogen oxides;
4. removal of soot.

These various functions can advantageously be arranged on a single multifunctional catalytic system, or on successive separate blocks. The sequence of these blocks depends on the architecture of the engine -post-combustion system selected by the designer. Depending on the emissions from the engine and in accordance with the current regulations, one or more of these functions may also be absent.

According to the invention, the various elements, namely the device for oxidation of carbon dioxide and oxidation of hydrocarbons (Cat-Ox) as well as the particulate filter (PAF) can be arranged differently, i.e. in a different position from that shown.

### Example 25

### Example of a Catalytic Converter of the Present Invention Incorporated in an Engine

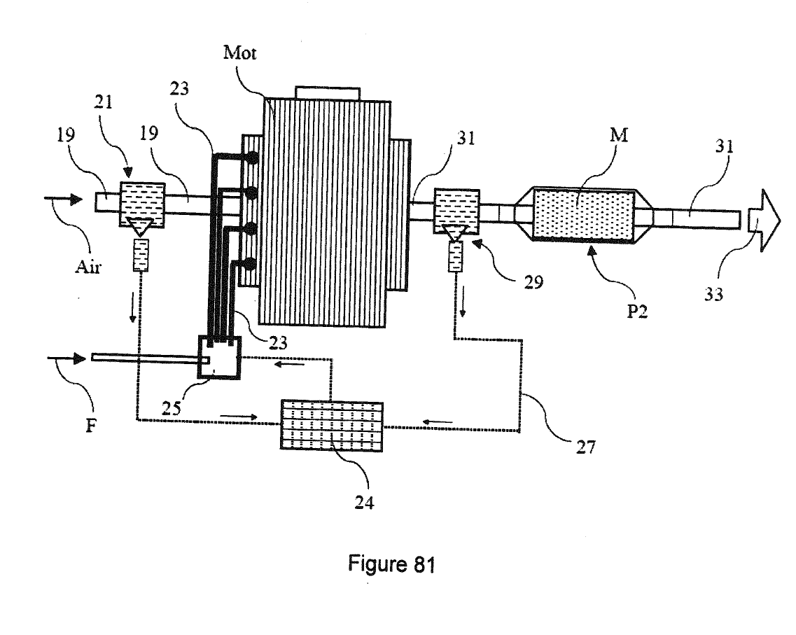

Another example of catalytic converter (P2) according to the present invention is shown in FIG. 81.

Various catalytic converters (P2) are constructed with a device according to the present invention described in examples 21 or 22.

The catalytic converter (P2) is connected to an engine (Mot). The engine is regulated by means of a control and post-injection system (24, 25, 27, 29).

The engine (Mot) is connected to an air admission pipe (19) and to a fuel admission pipe (F).

The post-injection system comprises a device for measuring the air flow rate (21) connected to a computer (24), a fuel admission pipe (F) connected to a device for regulating the flow rate of the fuel (25), said device for regulating the flow rate of the fuel is connected to the computer (24) and to the engine (Mot) by pipes and injectors (23). The line (27) connects the device for measuring the air flow rate (21) and a probe (29) for measuring the richness—level of CO and NO— of the exhaust gas, said probe being connected to the computer (24) via line (27). This system makes it possible to regulate the air flow rate and the fuel supply in relation to the richness of the exhaust gas.

The computer (24) controls the flow rates of air and fuel in order to obtain optimal combustion and a composition of the exhaust gases suitable for the functioning of the catalytic system.

The engine is also connected to a tube (31) for discharge of the exhaust gases comprising nitrogen oxides from the engine.

Downstream of the probe (29), a catalytic converter (P2) is arranged, comprising a ceramic honeycomb structure (M) according to the present invention comprising an MOF solid and means for contacting said MOF solid with the exhaust gas containing nitrogen oxides.

The system for removal of NOx described in this example also functions with a different architecture of the engine and/or of the equipment for admission, emission and control.

When the engine is running, the exhaust gas passes through the catalytic converter (P2) of the present invention and is thus treated by the MOF, which catalyzes removal of the nitrogen oxides.

Thus, the treated exhaust gas (33) no longer contains, or contains a small amount of nitrogen oxide, and the treated exhaust gas leaves via an exhaust pipe (31).

### REFERENCES

[1] www.senat.fr/rap/r05-125/r05-125.html
[2] www.doctissimo.fr/html/sante/mag_{—}2001/mag0817/dossier/sa_{—}440 4_pollution_effets_sante.htm
[3] P. Degobert, Automobile et Pollution, Editions Technip, Paris (1992).
[4] E. Samoli, E. Aga, G. Touloumi, K, Nisiotis, B. Forsberg, A. Lefranc, J. Pekkanen, B. Wojtyniak, C. Schindler, E. Niciu, R. Brunstein 1, M. Dodi Fikfak1, J. Schwartz, K. Katsouyanni, Eur. Respir. J., 27 (2006) 1129.
[5] Peters A; Liu E; Verrier R L; Schwartz J; Gold D R; Mittleman M; BMW J; Oh J A; Allen G; Monahan K; Dockery D W, Epidemiology (Cambridge, Mass.), (2000 January) Vol. 11, No. 1, pp. 11.
[6] C. Arden Pope, III; Richard T. Burnett; George D. Thurston; Michael J. Thun; Eugenia E. Calle; Daniel Krewski; John J. Godleski, Circulation, 109 (2004) 71.
[7] Gouriou F, Morin J-P, Weill M-E, Atmospheric Environment, 38 (2004) 2831.
[6] Son B, Yang W H, Breysse P, Chung W V, Lee Y S, Environmental Research, 94 (2004) 291.
[9] Praml G, Schierl R, International Archives of Occupational and Environmental Health, 73 (2000) 209.
[10] www.ademe.fr
[11] Miyata H, Konishi S, Ohno T, Hatayama F, J, Chem. Soc., Faraday Trans., 91 (1995) 149,
[12] Shelef M, McCabe R. W, Catal. Today, 62 (2000) 35.
[13] Farrauto R. J, Heck R. M, Catal. Today, 51 (1999) 351.
[14] Jobson E, Top. Catal., 28 (2004) 191.
[15] Pärvulescu V. I, Grange P, Delmont B, Catal. Today, 46 (1998) 233.
[16] Shelef M, Gandhi H. S, Ind. Eng. Chem. Prod. Res. Dev., 13 (1974) 80.
[17] Burch R, Coleman M. D, Appl. Catal. B, 23 (1999) 115.
[18] Held W, König A, Richter T, Puppe L, SAE paper no 900496, (1990).
[19] Iwamoto M, Proc. Meet. Catal. Technol. Renoval of NO, Tokyo, (1990) 17.
[20] Resini C, Montanari T, Nappil L, Bagnasco G, Turco M, Busca G, Bregani F, Notaro M, Rocchini G, J. Catal., 214 (2003) 179.
[21] Montanari T, Marie O, Daturi M, Busca G, Appl. Catal. B: Environ., 71 (2007) 216.
[22] Sato S, Yu-U Y, Yahiro H, Mizuno N, Iwamoto M, Appl. Catal., 70 (1991) L1.
[23] Hirabayashi H, Yahiro H, Mizuno N, Iwamoto M, Chem. Lett., (1992) 2235.
[24] Hamada H, Kintaichi Y, Sasaki M, Ito T, Appl. Catal., 64 (1990) L1.
[25] Kintaichi Y, Hamada H, Tabata M, Sasaki M, Ito T, Catal. Lett., 6 (1990) 239.
[26] Burch R, Millington J, Walker A. P, Appl. Catal. B, 4 (1994) 65.
[27] Burch R, Watling T. C, Appl. Catal. B, 11 (1997) 207.
[28] Obuchi A, Ohi A, Nakamura M, Ogata A, Mizuno K, Ohuchi H, App. Catal. B; Environ., 2 (1993) 71.
[29] Bamwenda G. R, Ogata A, Obuchi A, Oi J, Mizuno K, Skrzypek J, Appl. Catal. B Environ., 6 (1995) 311.
[30] Bourges P, Lunatti S, Mabilon G, Catalysis and automotive Pollution Control IV, Stud. Surf. Sci. Catal., 116 (1998) 213.
[31] Burch R, Ottery O, Appl. Catal. B, 9 (1996) L19.
[32] Tanaka T, Okuhara T, Misono M, Appl. Catal. B: Environ., 4 (1994) Li.
[33] Torikai Y, Yahiro H, Mizuno N, Iwamoto M, Catal. Lett., 9 (1991) 91
[34] Hamada H. Kintaichi Y, Sasaki M, Ito T, Tabata M, Appl. Catal., 75 (1991) L1.
Inaba M, Kintaichi Y, Hamada H, Proc. 1st Int. Cong. On Environ. Catal., (1995) 327.
[36] Miyadera T, Appl. Catal, B, 2 (1993) 199.
[37] Shimizu K, Satsuma A, Hattori T, Appl. Catal, B: Environ., 25 (2000) 239.
[38] Hamada H, Kintaichi Y, Sasaki M, Ito T, Yoshinari T, Appl. Catal. A General, 88 (1992) Li.
[39] Haneda M, Kintaichi Y, Hamada H, Catal. Lett., 55 (1998) 47.
[40] Busca G, Lietti L. Ramis G, Berti F, Appl. Catal. B: Environ., 18 (1998) 1.
[41] Busca G, La rubia M. A, Arrighi L, Ramis G, Catal. Today, 107-108 (2005) 139.
[42] Heck R. M, Catal, Today, 53 (1999) 519.
[43] Koebel M, Madia G, Elsener M, Catal. Today, 53 (1999) 519.
[44] Richter M, Trunschke A, Bentrup U, Brzezinka K. W, Schreier E, Schneider M, Pohl M.-M, Fricke R, J. Catal., 206 (2002) 98.
[45] Kato A, Matsuda S, Kamo T, Nakajima F, Kuroda H, Narita T, J. Phys. Chem., 85 (1981) 4099,
[46] Satterfield C. H, “Heterogenous Catalysis in Industrial Practice” Second edition. McGraw-Hill (1991).
[47] Toyota Patent EP 573 672A1 (1992).
[48] Miyoshi N, Matsumoto S, Katoh K, Tanaka T, Harada K, Takahashi N, Yokota K, Sugiura M, Kasahara K, SAE Technical Papers Series No. 950809, 1995.
[49] Krutzsch B, Wenninger G, Weibel M, Stapf P, Funk D. E, Webster E, Chaize E, Kasemo B, Martens J, Kienneman A, SAE Technical Papers Series No 982592 (1998).
[50] German Patent No. 4319 294 (1994).
[51.] Glick H, Klein J. J, Squire W, J. Chem. Eng. Prog. Ser., 27 (1971) 850.
[52] Fritz A, Pitchon V, Appl. Catal. B, 13 (1997) 1.
[53] Gomez-Garcia M. A, Pitchon V. Kiennemann A, Environment International 31 (2005) 445.
[54] Iwamoto M, Hamada M, Catal. Today, 10 (1991) 57.
[55] French Patent No. 98 05363.
[56] Hamada H, Kintaichi Y, Sasaki M, Ito T, Chem. Lett., 1991, 1069.
[57] Winter E. R. S, J. Catal., 22 (1971) 158.
[58] Amirnazmi A, Benson J. E, Boudart M, J. Catal., 30 (1973) 55
[59] Sato M, Surf. Sci., 95 (1980) 269.
[60] Sica A. M, Dos Santos J. H. Z, Baibich I. M, Gigola C. E, J. Mol. Cat. A: Chem., 137 (1999) 287,
[61] Neyertz C, Volpe M A, Gigola C, Catal. Today, 57 (2000) 255.
[62] Iwamoto M, Yokoo S, Sakai K, Kagawa S, J. Chem. Soc., Faraday Trans. 1, 77 (1981) 1629.
[63] Iwamoto M, Maruyama K, Yamasoe N, Seiyama T, J. Chem. Soc., Chem. Commun., (1972) 615.
[64] Lesage T, Verrier C, Bazin P, Saussey J, Daturi M, Phys. Chem. Chem. Phys., 5 (2003) 4435.
[65] Daturi M, Bion N, Saussey J, Lavalley J-C, Hedouin C, Seguelong T, Blanchard G, Phys. Chem. Chem. Phys., 3 (2001) 252.
[66] Bazin P, Marie O, Daturi M, Stud. Surf. Sci. Catal., 171 (2007) 97.
[67] C. Serre, F. Millange, S. Sulfaté, G. Férey Angew. Chem. Int. Ed. 2004, 43, 6286: A new route to the synthesis of trivalent transition metals porous carboxylates with trimeric SBU.
[68] Suzy Surblé, Christian Serre, Caroline Mellot-Draznieks, Franck Millange, and Gérard Férey: Chem, Comm. 2006, 284-286: A new isoreticular class of Metal-Organic-Frameworks with the MIL-88 topology.
[69] Thierry Loiseau, Ludovic Lecroq, Christophe Volkringer, Jérôme Marrot, Gérard Férey, Mohamed Haouas, Francis Taulelle, Sandrine Bourrelly, Philip L. Llewellyn and Michel Latroche: J. Am. Chem. Soc. 2006, 128, 10223: MIL-96, a Porous Aluminum Trimesate 3D Structure Constructed from a Hexagonal Network of 18-Membered Rings and i3-Oxo-Centered Trinuclear Units.
[70] Patricia Horcajada, Suzy Surblé, Christian Serre, Do-Young Hong, You-Kyong Seo, Jong-San Chang, Jean-Marc Grenéche, Irene Margiolaki and Gérard Férey, Chem Comm, 2007, 2820: Synthesis and catalytic properties of MIL-100(Fe), an iron(III) carboxylate with large pore.
[71] Gerard Férey, Caroline Mellot-Draznieks, Christian Serre, Franck Millange, Julien Dutour, Suzy Surblé, Irena Margiolaki: Science 2005 309, 2040: A Chromium Terephthalate-Based Solid with Unusually Large Pore Volumes and Surface Area.
[72] S. Surblé, F. Millange, C. Serre, T. Düren, M. Latroche, S. Bourrelly, P. L. Llewellyn and G. Férey J. Am. Chem. Soc. 128 (2006), 46, 14889. MIL-102: A Chromium Carboxylate Metal Organic Framework with Gas Sorption Analysis.
[73] K. Byrapsa, M. Yoshimura, <<Handbook of hydrothermal technology>>, Noyes Publications, Parkridge, N.J. USA, William Andrew Publishing, LLC, Norwich N.Y. USA, 2001
[74] G. Tompsett, W. C. Conner, K. S. Yngvesson, Chem Phys Chem. 2006, 7, 296
[75] S.-E. Park, J.-S. Chang, Y. K. Hwang, D. S. Kim, S. H. Jhung, J.-S. Hwang, Catal. Survey Asia 2004, 8, 91
[76] C. S. Cundy, Collect. Czech. Chem. Commum. 1998, 63, 1699
[77] S. H. Jhung, J.-H. Lee, J.-S. Chang, Bull. Kor. Chem., Soc. 2005, 26, 880
[78] A. Pichon, Cryst. Eng. Comm, 8, 2006, 211-214
[79] D. Braga, Angew. Chem. Int. Ed, 45, 2006, 142-246
[80] D. Braga, Dalton Trans., 2006, 1249-1263.
[81] Sabine Balthasar, Kerstin Michaelis, Norbert Dinauer, Hagen von Briesen, Jörg Kreuter and Klaus Langer, “Preparation and characterisation of antibody modified gelatin nanoparticles as drug carrier system for uptake in lymphocytes”, Biomaterials, 2005, 26, 15, 2723-2732.
[82] Ruxandra Gref, Patrick Couvreur, Gillian Barratt and Evgueni Mysiakine, “Surface-engineered nanoparticles for multiple ligand coupling”, Biomaterials, 2003, 24, 24, 4529-4537,
[83] Gérard Férey, Christian Serre, Caroline Mellot-Draznieks, Franck Millange, Suzy Surblé, Julien Dutour, Irena Margiolaki Angew. Chem. Int. Ed. 2004, 43, 6296: Access to a giant pores hybrid solid (>380,000 Å³) by combination of mastered chemistry, simulation and powder diffraction.
[84] A. Vimont, J.-M. Goupil, J.-C. Lavalley, and M. Daturi, S. Surblé, C. Serre, F. Millange, G. Férey, N. Audebrand, J. Am. Chem. Soc., 2006, 128, 3218-3227: First characterization of acid sites in a new chromium(III) dicarboxylate with giant pores.
[85] J. Chatt and L. A. Duncanson, J. Chem. Soc., 1953, 2939-2947: Olefin co-ordination compounds. Part III. Infra-red spectra and structure: attempted preparation of acetylene complexes.
[86] A. Vimont, H. Leclerc, F. Mauge, M. Daturi, J. C. Lavalley, S. Surblé, C. Serre et G. Férey, Journal of Physical Chemistry C, 111 (2007), 383-388: Creation of Controlled Bronsted Acidity on a Zeotypic Mesoporous Chromium(III) Carboxylate by Grafting Water and Alcohol Molecules.
[87] C. T. Dziobkowski, T. J. Wrobleski, D. B. Brown, Inorg. Chem. 1982, 20, 671.
[88] S. Ameerunisha et al., J. Chem. Soc. Perkin Trans. 2 1995, 1679.
[89] Y. Liu, J. F. Eubank, A. J. Cairns, J. Eckert, V. Ch. Kravtsov, R. Luebke, M. Eddaoudi, Angew. Chem. Int. Ed. 2007, 46, 3278-3283.
[90] Shiotani Akinori, Z. Naturforsch. 1994, 49, 12, 1731-1736.
[91] Serre et al., Science, 2007, 315, 1828.
[92] C. Serre et al. J. Am. Chem. Soc., 2005, 127, 16273-16278.
[93] B. N. Shelimov, N. N. Tolkachev, O. P. Tkachenko, G. N. Baeva, K. V. Klementiev, A. Yu. Stakheev, V. B. Kazansky, Journal of Photochemistry and Photobiology A-Chemistry, 195 (2008) 81.
[94] T. R. Whitfield, X. Wang, L. Liu, A. J. Jacobson Metal-organic frameworks based on iron oxide octahedral chains connected by benzenedicarboxylate dianions, Solid State Sci., 7, 1096-1103, 2005.
[95] Handbook of Heterogeneous Catalysis, 2nd Edition, G. Ertl, H. Knözinger, F. Schüth, J. Weitkamp Editors, 2008, ISBN: 978-3-7-31241-2.
[96] S. S.-Y. Chui et al., Science, 283, 1148-1150: A chemically functionalizable material [Cu₃(TMA)O₂O)₃]ₙ,
[97] P. G. M Wuts & T. W. Greene, <<Greene's Protective Groups in organic Synthesis>>, fourth edition, 2007, Editions John Wiley & Son,

1. A Nitrogen oxide reduction catalyst comprising a porous crystalline MOF solid consisting of a three-dimensional succession of units corresponding to the following formula (I):
MₘOₖXₗLₚ (I)
where, in formula (I):
each occurrence of M represents independently a metal cation M selected from the group comprising Al³⁺, Ca²⁺, Cu⁺, Cu²⁺, Cr³⁺, Fe²⁺, Fe³⁺, Ga³⁺, Mg²⁺, Mn²⁺, Mn³⁺, Mn⁴⁺, Ti³⁺, Ti⁴⁺, V³⁺, V⁴⁺, Zn²⁺, Zn³⁺, Zr⁴⁺, Ln³⁺ in which Ln is a rare earth;
in is 1 to 12;
k is 0 to 4;
l is 0 to 18;
p is 1 to 6;
X is an anion selected from the group comprising OH⁻, Cl⁻, F₋, I⁻, Br⁻, SO₄²⁻, NO₃⁻, ClO4⁻, PF₆⁻, BF₄⁻, R—(COO)ₙ⁻ where R is as defined below, R¹—(COO)ₙ⁻, R¹—(SO₃)ₙ⁻, R₁—(PO₃)ₙ⁻, where R¹ is a hydrogen, a linear or branched, optionally substituted C₁-C₁₂ alkyl, an aryl, where n is an integer from 1 to 4;
L is a spacer ligand comprising a radical R having q 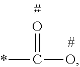
carboxylate groups where
q is 1, 2, 3, 4, 5 or 6; denotes the point of attachment of the carboxylate to the radical R;
# denotes the possible points of attachment of the carboxylate to the metal ion;
R represents:
(i) a C₁₋₁₂alkyl, C₂₋₁₂alkenyl or C₂₋₁₂alkynyl radical;
(ii) a fused or unfused, mono- or polycyclic aryl radical comprising 6 to 50 carbon atoms;
(iii) a fused or unfused, mono- or polycyclic heteroaryl comprising 1 to 50 carbon atoms;
(iv) an organic radical comprising a metallic element selected from the group comprising ferrocene, porphyrin, phthalocyanine;
the radical R optionally being substituted with one or more groups R², selected independently from the group comprising C₁₋₁₀alkyl; C₂₋₁₀alkenyl; C₂₋₁₀alkynyl; C₃₋₁₀cycloalkyl; C₁₋₁₀heteroalkyl; C₁₋₁₀haloalkyl; C₆₋₁₀aryl; C₃₋₂₀heterocyclic; C₁₋₁₀alkylC₆₋₁₀aryl; C₁₋₁₀alkylC₃₋₁₀heteroaryl; F; Cl; Br; I; —NO₂; —CN; —CF₃; —CH₂CF₃; —OH; —CH₂OH; —CH₂CH₂OH; —NH₂; —CH₂NH₂; —NHCHO; —COOH; —CONH₂; —SO₃H; —CH₂SO₂CH₃; —PO₃H₂; or a function -GR^{G1} in which G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, where each occurrence of R^{G2} is, independently of the other occurrences of R^{G2}, a hydrogen atom; or a C₁₋₁₂alkyl, C₁₋₁₂heteroalkyl, C₂₋₁₀alkenyl or C₂₋₁₀alkynyl function, linear, branched or cyclic, optionally substituted; or a C₆₋₁₀aryl, C₃₋₁₀heteroaryl, C₅₋₁₀heterocyclic, C₁₋₁₀alkylC₆₋₁₀aryl or C₁₋₁₀alkylC₃₋₁₀heteroaryl group in which the aryl, heteroaryl or heterocyclic radical is optionally substituted; or else, when G represents —NR^{G2}—, R^{G1} and R^{G2}, together with the nitrogen atom to which they are bound, form a heterocycle or a heteroaryl, optionally substituted.

2. The catalyst as claimed in claim 1, in which the ligand L is a di-, tri-, tetra- or hexa-carboxylate ligand selected from the group comprising fumarate, succinate, glutarate, muconate, adipate, 2,5-thiophenedicarboxylate, terephthalate, 2,5-pyrazine dicarboxylate, naphthalene-2,6-dicarboxylate, biphenyl-4,4′-dicarboxylate, azobenzenedicarboxylate, dichloroazobenzenedicarboxylate, azobenzenetetracarboxylate, dihydroxoazobenzenedicarboxylate, benzene-1,2,4-tricarboxylate, benzene-1,3,5-tricarboxylate, benzene-1,3,5-tribenzoate, 1,3,5-tris[4′-carboxy(1,1′-biphenyl-4-yl)benzene, benzene-1,2,4,5-tetracarboxylate, naphthalene-2,3,6,7-tetracarboxylate, naphthalene-1,4,5,8-tetracarboxylate, biphenyl-3,5,3′,5′-tetracarboxylate, and modified analogs selected from the group comprising 2-aminoterephthalate, 2-nitroterephthalate, 2-methylterephthalate, 2-chloroterephthalate, 2-bromoterephthalate, 2,5-dihydroxoterephthalate, tetrafluoroterephthalate, 2,5-dicarboxyterephthalate, dimethyl-4,4′-biphenydicarboxylate, tetramethyl-4,4′-biphenydicarboxylate, dicarboxy-4,41-biphenydicarboxylate.

3. The catalyst as claimed in claim 1, in which the anion X is selected from the group comprising OH⁻, Cl⁻, F⁻, R—(COO)ₙ⁻, PF₆⁻, ClO₄⁻, with R and n as defined in claim 1.

4. The catalyst as claimed in claim 1, comprising a percentage by weight of N in the dry phase from 5 to 50%.

5. The catalyst as claimed in claim 1, in which the pore size of the MOF material is from 0.4 to 6 nm.

6. The catalyst as claimed in claim 1, in which the solid has a gas loading capacity from 0.5 to 50 mmol of gas per gram of dry solid.

7. The catalyst as claimed in claim 1, in which at least 1 to 5 mmol of gas per gram of dry solid is coordinated with M.

8. The catalyst as claimed in claim 1, in which said solid has a flexible structure that swells or shrinks with an amplitude in the range from 10 to 300%.

9. The catalyst as claimed in claim 1, in which said solid has a rigid structure that swells or shrinks with an amplitude in the range from 0 to 10%.

10. The catalyst as claimed in claim 1, in which the solid has a pore volume from 0.5 to 4 cm³/g.

11. The catalyst as claimed in claim 1, in which M is an Fe ion.

12. The catalyst as claimed in claim 3, in which said solid comprises a three-dimensional succession of units corresponding to formula (I) selected from the group comprising:
Fe₃OX [O₂C—C₂H₂—CO₂]₃ of flexible structure
Fe₃OX [O₂C—C₆H₄—CO₂]₃ of flexible structure
Fe₃OX [O₂C—C₁₀H₆—CO₂]₃ of flexible structure
Fe₃OX [O₂C—C₁₂H₈—CO₂]₃ flexible structure
Fe₃OX [O₂C—C₄H₄—CO₂]₃ of flexible structure
Fe(OH) [O₂C—C₄H₄—CO₂] of flexible structure
Fe₁₂O(OH)₁₀ (H₂O)₃[C₆H₃—(CO₂)₃]₆ of rigid structure
Fe₃OX [C₆H₃—(CO₂)₃]₂ of rigid structure
Fe₃OX [O₂C—C₆H₄—CO₂]₃ of rigid structure
Fe₆O₂X₂[C₁₀H₂—(CO₂)₄]₃ of rigid structure
Fe₆O₂X₂[C₁₄H₂—(CO₂)₄]₃ of rigid structure.

13. The catalyst as claimed in claim 1, in which the nitrogen oxide is in the form of NO or NO₂ or N₂O or of a mixture of two or of three of the latter.

14. The catalyst as claimed in claim 1, comprising a step of contacting said MOF solid with the nitrogen oxide to be reduced.

15. The catalyst as claimed in claim 14, comprising, before the contacting step, a step of activation of the MOF solid by heating under vacuum or under reducible or neutral atmosphere.

16. The catalyst as claimed in claim 15, in which, in the activation step, heating is carried out at a temperature from 150 to 280° C.

17. The catalyst as claimed in claim 14, in which the contacting is carried out in the presence of oxygen and/or water.

18. A method for removing nitrogen oxide from a medium comprising contacting the medium with a catalyst comprising a porous crystalline MOF solid consisting of a three-dimensional succession of units corresponding to the following formula (I):
MₘOₖXₗLₚ (I)
where, in formula (I):
each occurrence of M represents independently a metal cation M selected from the group comprising Al³⁺, Ca²⁺, Cu⁺, Cu²⁺, Cr³⁺, Fe²⁺, Fe³⁺, Ga³⁺, Mg²⁺, Mn²⁺, Mn³⁺, Mn⁴⁺, Ti³⁺, Ti⁴⁺, V³⁺, V⁴⁺, Zn²⁺, Zn³⁺, Zn⁴⁺, Ln³⁺ in which Ln is a rare earth;
m is 1 to 12;
k is 0 to 4;
l is 0 to 18;
p is 1 to 6;
X is an anion selected from the group comprising OH⁻, Cl⁻, F⁻, I⁻, Br⁻, SO₄²⁻, NO₃⁻, ClO4⁻, PF₆⁻, BF₄⁻, R—(COO)ₙ⁻ where R is as defined below, R¹—(COO)ₙ⁻, R¹—(SO₃)ₙ⁻, R¹—(PO₃)ₙ⁻, where R¹ is a hydrogen, a linear or branched, optionally substituted C₁-C₁₂ alkyl, an aryl, where n is an integer from 1 to 4;
L is a spacer ligand comprising a radical R having q 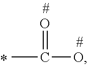
carboxylate groups ₁-Co, where
q is 1, 2, 3, 4, 5 or 6; * denotes the point of attachment of the carboxylate to the radical R;
# denotes the possible points attachment of the carboxylate to the metal ion;
R represents:
(i) a C₁₋₁₂alkyl, C₂₋₁₂alkenyl or C₂₋₁₂alkynyl radical;
(ii) a fused or unfused, mono- or polycyclic aryl radical comprising 6 to 50 carbon atoms;
(iii) a fused or unfused, mono- or polycyclic heteroaryl comprising 1 to 50 carbon atoms;
(iv) an organic radical comprising a metallic element selected from the group comprising ferrocene, porphyrin, phthalocyanine;
the radical R optionally being substituted with one or more groups R², selected independently from the group comprising C₁₋₁₀alkyl; C₂₋₁₀alkenyl; C₂₋₁₀alkynyl; C₃₋₁₀cycloalkyl; C₁₋₁₀heteroalkyl; C₁₋₁₀haloalkyl; C₆₋₁₀aryl; C₃₋₂₀heterocyclic; C₁₋₁₀alkylC₆₋₁₀aryl; C₁₋₁₀alkylC₃₋₁₀heteroaryl; F; Cl; Br; I; —NO₂; —CN; —CF₃; —CH₂CF₃; —OH; —CH₂OH; —CH₂CH₂OH; —NH₂; —CH₂NH₂; —NHCHO; —COOH; —CONH₂; —SO₃H; —CH₂SO₂CH₃; —PO₃H₂; or a function -GR^{G1} in which G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, where each occurrence of R^{G2} is, independently of the other occurrences of R^{G2}, a hydrogen atom; or a C₁₋₁₂alkyl, C₁₋₁₂heteroalkyl, C₂₋₁₀alkenyl or C₂₋₁₀alkynyl function, linear, branched or cyclic, optionally substituted; or a C₆₋₁₀aryl, C₃₋₁₀heteroaryl, C₅₋₁₀heterocyclic, C₁₋₁₀alkylC₆₋₁₀aryl or C₁₋₁₀alkylC₃₋₁₀heteroaryl group in which the aryl, heteroaryl or heterocyclic radical is optionally substituted; or else, when G represents —NR^{G2}—, R^{G1} and R^{G2}, together with the nitrogen atom to which they are bound, form a heterocycle or a heteroaryl, optionally substituted.

19. The method as claimed in claim 18, in which the medium is a liquid or gaseous effluent.

20. The method as claimed in claim 19, in which the effluent comes from combustion of hydrocarbons or from oxidation of nitrogen compounds.

21. The method as claimed in claim 20, in which the effluent is selected from an effluent from a vehicle, boat, factory, workshop, laboratory, stored products, urban air vents.

22. The catalyst as claimed in claim 1, in which the MOF solid is in a form selected from nanoparticles, a powder, pebbles, granules, a coating.

23. A device for removing nitrogen oxide, said device comprising an MOF solid as defined in claim 1, and means for contacting said MOF solid with the nitrogen oxide.

24. The device as claimed in claim 23, in which the means for contacting the MOF solid with the nitrogen oxide are means for bringing the MOF solid into contact with a liquid or gaseous effluent comprising said nitrogen oxide.

25. The device as claimed in claim 23, in which the MOF solid is in a form selected from nanoparticles, a powder, pebbles, granules, pellets, a coating.

26. The device as claimed in claim 24, in which the effluent is selected from water, a vehicle exhaust gas, liquid and gaseous effluents from a factory, from a workshop, from a laboratory, from stored products, from an urban aeration intake, from air conditioning, from an air purifier, said device permitting contact of the MOF solid with the effluent for removing the nitrogen oxide therefrom.

27. The device as claimed in claim 24, in which the MOF solid is in a form selected from nanoparticles, a powder, pebbles, granules, pellets, a coating.
